SEQUENCE LISTING (37 sequences)

SEQ ID NO: 1 - protein, 305 aa, Escherichia coli
MTWLPLNPIPLKDRVSMIFLQYGQIDVIDGAFVLIDKTGIRTHIPVGSVACIMLEPGTRVSHAAVRLAAQVGTLLVWVGEAGVRVYASGQPGGARSDKLLYQAKLALDEDLRLKVVRKMFELRFGEPAPARRSVEQLRGIEGSRVRATYALLAKQYGVTWNGRRYDPKDWEKGDTINQCISAATSCLYGVTEAAILAAGYAPAIGFVHTGKPLSFVYDIADIIKFDTVVPKAFEIARRNPGEPDREVRLACRDIFRSSKTLAKLIPLIEDVLAAGEIQPPAPPEDAQPVAIPLPVSLGDAGHRSS

SEQ ID NO: 2 - protein, 94 aa, Escherichia coli [sequence omitted]

SEQ ID NO: 3 - DNA, 90 bp, artificial sequence
  Synthetic
ggaagagagtcaattcagggtggtgaatgtgaaaccagtatagtgataagatgtcgcagagtatgccggtgtctcttatcagaccgtttc

SEQ ID NO: 4 - DNA, 90 bp, artificial sequence
  Synthetic
  misc_feature (42)..(49): n is a, c, g, or t
ggggaacacccgtaagtggtttgagcgatgatatttgtgctnnnnnnnnccccgctggcgcggggaacactctaaacataacctattatt

SEQ ID NO: 5 - DNA, 20 bp, artificial sequence
  Synthetic
gcgagcgatccagaagatct

SEQ ID NO: 6 - DNA, 21 bp, artificial sequence
  Synthetic
gggtaaaggatgccacagaca

SEQ ID NO: 7 - DNA, 22 bp, artificial sequence
  Synthetic
cgctctatgatccagtcgattt

SEQ ID NO: 8 - DNA, 20 bp, artificial sequence
  Synthetic
tccgtatgccatgcgtttat

SEQ ID NO: 9 - DNA, 79 bp, artificial sequence
  Synthetic
aatgatacggcgaccaccgagatctacactagatcgcctggcttaaaaaatcattaattaataataggttatgtttaga

SEQ ID NO: 10 - DNA, 79 bp, artificial sequence
  Synthetic
aatgatacggcgaccaccgagatctacacctctctatctggcttaaaaaatcattaattaataataggttatgtttaga

SEQ ID NO: 11 - DNA, 79 bp, artificial sequence
  Synthetic
aatgatacggcgaccaccgagatctacactatcctctctggcttaaaaaatcattaattaataataggttatgtttaga

SEQ ID NO: 12 - DNA, 79 bp, artificial sequence
  Synthetic
aatgatacggcgaccaccgagatctacacagagtagactggcttaaaaaatcattaattaataataggttatgtttaga

SEQ ID NO: 13 - DNA, 79 bp, artificial sequence
  Synthetic
aatgatacggcgaccaccgagatctacacgtaaggagctggcttaaaaaatcattaattaataataggttatgtttaga

SEQ ID NO: 14 - DNA, 79 bp, artificial sequence
  Synthetic
aatgatacggcgaccaccgagatctacacactgcatactggcttaaaaaatcattaattaataataggttatgtttaga

SEQ ID NO: 15 - DNA, 79 bp, artificial sequence
  Synthetic
aatgatacggcgaccaccgagatctacacaaggagtactggcttaaaaaatcattaattaataataggttatgtttaga

SEQ ID NO: 16 - DNA, 79 bp, artificial sequence
  Synthetic
aatgatacggcgaccaccgagatctacacctaagcctctggcttaaaaaatcattaattaataataggttatgtttaga

SEQ ID NO: 17 - DNA, 56 bp, artificial sequence
  Synthetic
caagcagaagacggcatacgagattcgccttaggtttgagcgatgatatttgtgct

SEQ ID NO: 18 - DNA, 56 bp, artificial sequence
  Synthetic
caagcagaagacggcatacgagatctagtacgggtttgagcgatgatatttgtgct

SEQ ID NO: 19 - DNA, 56 bp, artificial sequence
  Synthetic
caagcagaagacggcatacgagatttctgcctggtttgagcgatgatatttgtgct

SEQ ID NO: 20 - DNA, 56 bp, artificial sequence
  Synthetic
caagcagaagacggcatacgagatgctcaggaggtttgagcgatgatatttgtgct

SEQ ID NO: 21 - DNA, 56 bp, artificial sequence
  Synthetic
caagcagaagacggcatacgagataggagtccggtttgagcgatgatatttgtgct

SEQ ID NO: 22 - DNA, 56 bp, artificial sequence
  Synthetic
caagcagaagacggcatacgagatcatgcctaggtttgagcgatgatatttgtgct

SEQ ID NO: 23 - DNA, 56 bp, artificial sequence
  Synthetic
caagcagaagacggcatacgagatgtagagagggtttgagcgatgatatttgtgct

SEQ ID NO: 24 - DNA, 56 bp, artificial sequence
  Synthetic
caagcagaagacggcatacgagatcctctctgggtttgagcgatgatatttgtgct

SEQ ID NO: 25 - DNA, 56 bp, artificial sequence
  Synthetic
caagcagaagacggcatacgagatagcgtagcggtttgagcgatgatatttgtgct

SEQ ID NO: 26 - DNA, 56 bp, artificial sequence
  Synthetic
caagcagaagacggcatacgagatcagcctcgggtttgagcgatgatatttgtgct

SEQ ID NO: 27 - DNA, 56 bp, artificial sequence
  Synthetic
caagcagaagacggcatacgagattgcctcttggtttgagcgatgatatttgtgct

SEQ ID NO: 28 - DNA, 56 bp, artificial sequence
  Synthetic
caagcagaagacggcatacgagattcctctacggtttgagcgatgatatttgtgct

SEQ ID NO: 29 - DNA, 58 bp, artificial sequence
  Synthetic
ctggcttaaaaaatcattaattaataataggttatgtttagagtgttccccgcgccag

SEQ ID NO: 30 - DNA, 37 bp, artificial sequence
  Synthetic
cggggataaaccgagcacaaatatcatcgctcaaacc

SEQ ID NO: 31 - DNA, 285 bp, artificial sequence
  Synthetic
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatactacctgacgctttttatcgcaactctctactgtttctccat

SEQ ID NO: 32 - DNA, 4755 bp, artificial sequence
  Synthetic
ggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccagatatcgacgtctaagaaaccattattatcatgacattaacctataaaaataggcgtatcacgaggccctttcgtcttcacctcgagtcggtgatgtcggcgatataggcgccagcaaccgcacctgtggcgccggtgatgccggccacgatgcgtccggcgtagaggatctgctcatgtttgacagcttatcatcgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagagaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatactacctgacgctttttatcgcaactctctactgtttctccatacccgtttttttgggctagaaataattttgtttaactttaagaaggagatatacatatgcggggttctcatcatcatcatcatcatggtatggctagcatgactggtggacagcaaatgggtcgggatctgtacgagaacctgtacttccaggaggacgccttatgacctggcttccccttaatcccattccactcaaagatcgcgtctccatgatctttctgcaatatgggcagatcgatgtaatagatggcgcgtttgtacttatcgacaagacagggatccgcactcatattcctgttggctcggttgcctgcatcatgctggaacctggtacacgggtttcgcatgcagctgtacgcctggctgcgcaagttggaacattgttggtatgggtgggggaagcgggcgttcgtgtttatgcttctggtcagcctggaggtgcgcgttcagataagctgctctatcaggcaaaacttgctctggatgaagatttgcgtctgaaggtcgtacgtaaaatgtttgaacttcggtttggagaacctgcgcctgcccggcgctccgtagagcaactcagaggtatagaaggcagtcgcgtgcgggcaacctacgcacttctggcgaagcaatacggcgtgacatggaatggacgtcgctacgatccgaaagactgggaaaagggcgatacgatcaaccaatgcattagcgctgcaacttcctgtttatacggcgtaactgaagcggcgatacttgcagctggttatgcaccagctattgggtttgtgcatacaggaaagcctctttcctttgtttacgatattgcagacatcattaaatttgacactgttgtaccgaaagcttttgagatagcgcgtcgtaaccctggtgagccggaccgggaagtccgtttggcgtgcagggatatttttcgcagtagtaaaacattagccaaattgattccgcttatagaggacgtgcttgccgctggagaaatacaaccgccggccccacctgaagatgcacagcctgttgccattccgcttcctgtttcactgggagatgcaggccatcggagtagctgaaatgagtatgttggtcgtggtcactgaaaatgtacctccgcgcttacgaggcagattagccatctggttgttggaggtacgtgcaggggtatatgtaggtgatgtatccgcaaaaattcgtgaaatgatctgggaacaaatagctggactggcggaagaaggcaatgtagtgatggcatgggcaacgaatacggaaacgggatttgagttccagacatttgggttaaacaggcgtaccccggtagatttggatggtttaaggttggtgtcttttttacctgtttgataataatctagaccaggcatcaaataaaacgaaaggctcagtcgaaagactgggcctttcgttttatctgttgtttgtcggtgaacgctctctactagagtcacactggctcaccttcgggtgggcctttctgcgtttataggtacccgttgagagaagattttcagcctgatacagattaaatctagacctaggcgttcggctgcggcgagcggtatcagctcactcaaaggcggtaatacggttatccacagaatcaggggataacgcaggaaagaacatgtgagcaaaaggccagcaaaaggccaggaaccgtaaaaa

SEQ ID NO: 33 - DNA, 7 bp, artificial sequence
  Synthetic
atggtcc

SEQ ID NO: 34 - DNA, 7 bp, artificial sequence
  Synthetic
acatcag

SEQ ID NO: 35 - protein, 305 aa, artificial sequence
  Synthetic
MTWLPLNPILLKDRVSMIFLQYGQIDVIDGAFVLIDKTGIRTHIPVGSVACIMLEPGTRVSHAAVRLAAQVGTLLVWVGEAGVRVYASGQPGGARSDKLLYQAKLALDEDLRLKVVRKMFELRFGEPAPARRSVEQLRGIEGSRVRATYALLAKQYGVTWNGRRYDPKDWEKGDTINQCISAATSCLYGVTEAAILAAGYAPAIGFVHTGKPLSFVYDIADIIKFDTVVPKAFEIARRNPGEPDREVRLACRDIFRSSKTLAKLIPLIEDVLAAGEIQPPAPPEDAQPVAIPLPVSLGDAGHRSS

SEQ ID NO: 36 - protein, 94 aa, artificial sequence
  Synthetic
MSMLVVVTENVPPRLRGRLAIWLLEVRAGVYVGDVSAKIREMIWEQIAGLAGEGNVVMAWATNTETGFEFQTFGLNRRTPVDLDGLRLVSFLPV

SEQ ID NO: 37 - DNA, 7 bp, artificial sequence
  Synthetic
ataaacc

(19) United States
(12) Patent Application Publication
(10) Pub. No.: US 2022/0403419 A1
(43) Pub. Date: Dec. 22, 2022

(54) CRISPR-BASED METHODS FOR RECORDING BIOLOGICAL SIGNALS

(71) Applicant: THE TRUSTEES OF COLUMBIA UNIVERSITY IN THE CITY OF NEW YORK, New York, NY (US)

(72) Inventors: Harris He Wang, New York, NY (US); Ravi Sheth, New York, NY (US); Christian Munck, New York, NY (US); Sung Sun Yim, New York, NY (US)

(21) Appl. No.: 17/777,557

(22) PCT Filed: Nov. 18, 2019
(86) PCT No.: PCT/US2020/060973

§ 371 Date: May 17, 2022

Related U.S. Application Data
(60) Provisional application No. 62/937,029, filed on Nov. 18, 2019.

(51) Int. Cl. C12N 15/90; C12N 15/11; C12N 9/22

(52) U.S. Cl. CPC C12N 15/907; C12N 15/11; C12N 9/22; C12N 2310/20; C12N 2800/80

(57) ABSTRACT

Provided herein are methods and systems to record temporal biological signals into the genomes of engineered cells (e.g., genomes of a bacterial population) using the CRISPR-Cas system.

### CROSS-REFERENCE TO RELATED APPLICATIONS

This application claims the benefit of U.S. Provisional Application No. 62/937,029, filed Nov. 18, 2019, the contents of each of which are incorporated herein by reference.

### FIELD OF THE INVENTION

The present invention relates to CRISPR-based methods and systems for recording temporal biological signals using engineered cells.

### BACKGROUND

DNA is the primary information storage medium in living organisms and can be utilized in synthetic cellular memory devices that convert biological signals into heritable changes in nucleotide sequences. For example, approaches using recombinases, single-stranded DNA recombineering, and CRISPR-Cas9 have been developed to record the level of a biological signal or to track developmental lineage. However, a major outstanding challenge is the robust recording of temporally varying biological states or signals (e.g. gene expression, metabolite fluctuations) in living cells. Such a biological recording system would have powerful applications in studying dynamic cellular processes including complex regulatory programs, or engineering “sentinel” cells that track changing environmental signals over time.

The bacterial CRISPR-Cas adaptation process exemplifies a naturally occurring biological memory system. When foreign genetic elements such as plasmids and phages invade a cell, short fragments of these exogenous nucleic acids can be captured by CRISPR-Cas adaptation proteins and integrated into genomic CRISPR arrays as spacers. This spacer acquisition process occurs in a unidirectional manner; new spacers are inserted at the 5′ of CRISPR arrays and can be subsequently used by CRISPR-Cas immunity proteins to repel future invaders that exhibit matching sequence identity. The DNA writing potential of the adaptation process has been recently explored to record the sequence and ordering of chemically synthesized oligonucleotides that were serially electroporated into cell populations. However, engineering the CRISPR-Cas adaptation system to directly record biological signals and their temporal context, and not simply sequence information of exogenous DNA, has not been achieved to-date.

There is still a need to robustly and accurately profile time-varying biological signals and regulatory programs. The present disclosure provides for a scalable strategy to record temporal biological signals into genomes of a bacterial population using the CRISPR-Cas adaptation system.

### SUMMARY OF THE INVENTION

The present disclosure provides for a method of recording a temporal biological signal in an engineered, non-naturally occurring cell, comprising: exposing the cell to a temporal biological signal, wherein the cell comprises a trigger nucleic acid and a CRISPR-Cas system, wherein the CRISPR-Cas system comprises a CRISPR array nucleic acid sequence, wherein the trigger nucleic acid comprises at least one oligonucleotide spacer, wherein presence and/or strength of the temporal biological signal correlates with an abundance of the oligonucleotide spacer, wherein the CRISPR-Cas system unidirectionally inserts the oligonucleotide spacer into the CRISPR array nucleic acid sequence, and wherein the abundance of the oligonucleotide spacers correlates with a frequency of the oligonucleotide spacer inserted into the CRISPR array nucleic acid sequence.

The present disclosure also provides for a method of recording a plurality of temporal biological signals in engineered, non-naturally occurring cells, comprising:
(a) mixing a plurality of populations of cells to generate mixed cells, each population of cells comprising a trigger nucleic acid and a CRISPR-Cas system, wherein the CRISPR-Cas system comprises an CRISPR array nucleic acid sequence, wherein the trigger nucleic acid comprises one or more oligonucleotide spacers, wherein the oligonucleotide spacers in different populations of cells differ; and
(b) exposing the mixed cells to a plurality of temporal biological signals,
   wherein presence and/or strength of each temporal biological signal correlates with an abundance of a corresponding oligonucleotide spacer,
   and wherein the CRISPR-Cas system unidirectionally inserts the oligonucleotide spacer into the CRISPR array nucleic acid sequence, wherein the abundances of the oligonucleotide spacers correlate with frequencies of the oligonucleotide spacers inserted into the CRISPR array nucleic acid sequence.

In certain embodiments, the oligonucleotide spacers are barcoded via a nucleic acid sequence of a direct repeat (DR) of the CRISPR array nucleic acid sequence.

The present disclosure also provides for a method of reconstructing lineage of cells, comprising: analyzing a sequence identity of a plurality of reference spacers inserted into a CRISPR array nucleic acid sequence in the cells, wherein the cells comprise a CRISPR-Cas system comprising the CRISPR array nucleic acid sequence.

In certain embodiments, the CRISPR-Cas system inserts one or more reference spacers into the CRISPR array nucleic acid sequence.

In certain embodiments, the reference spacers are derived from the cell's genome and/or one or more plasmids in the cell.

Also encompassed by the present disclosure is a biological recording system comprising: an engineered, non-naturally occurring cell comprising a trigger nucleic acid and a CRISPR-Cas system, wherein the CRISPR-Cas system comprises an CRISPR array nucleic acid sequence, wherein the trigger nucleic acid comprises at least one oligonucleotide spacer, wherein an abundance of the oligonucleotide spacer is increased by presence and/or strength of a temporal biological signal, wherein the CRISPR-Cas system unidirectionally inserts the oligonucleotide spacer into the CRISPR array nucleic acid sequence, and wherein the abundance of the oligonucleotide spacer correlates with a frequency of the oligonucleotide spacer inserted into the CRISPR array nucleic acid sequence.

In certain embodiments, a copy number of the trigger nucleic acid is increased by presence and/or strength of a temporal biological signal.

In certain embodiments, the trigger nucleic acid is a plasmid.

In certain embodiments, the cell is a prokaryotic cell or a eukaryotic cell. In certain embodiments, the prokaryotic cell is a bacterial cell, such as Escherichia coli.

In certain embodiments, the eukaryotic cell is a yeast cell, plant cell or a mammalian cell such as a human cell.

In certain embodiments, the CRISPR array nucleic acid sequence resides in a genomic DNA of the cell or on a plasmid.

In certain embodiments, the signal is a gene expression signal, a metabolite/substance concentration signal, a photo-activated signal, a light-induced signal, a transcriptional signal, a molecular interaction signal, a receptor modulation signal, an electrical signal, and/or an environment signal.

In certain embodiments, the recorded temporal biological signal is reconstructed. In certain embodiments, the reconstructing is by sequencing the CRISPR array nucleic acid sequence. In one embodiment, the sequencing determines sequence and order of inserted oligonucleotide spacers in the CRISPR array nucleic acid sequence.

In certain embodiments, the CRISPR-Cas system comprises Cas1 and/or Cas2.

The present disclosure provides for a kit comprising the present biological recording system.

In other embodiments, improvements to various aspects of the CRISPR-Cas recording system have been devised to improve performance and range including: improving efficiency of spacer incorporation from 10% to 50%; increasing temporal resolution from hours to minutes; increasing the duration of recording from days to weeks; demonstrating portability to other microbes beyond E. coli BL21; and expanding to new recording modalities including chemicals, electrical, light, etc.

Improvements to the system include the implementation of a promoter, Pbad, into the Cas1-2 containing plasmid to drive expression of Cas1-2 based on presence of arabinose. As is shown in FIG. 24, use of the Pbad promoter increases CRISPR-Cas recording in many different clinically related isolates, include K. pneumoniae (KP08), as well as EC77 and BL21.

Accordingly, in a further embodiment, provided is a plasmid comprising a sequence encoding Cas1, a sequence encoding Cas2 and a sequence encoding a Pbad promoter upstream of the Cas1 and Cas2 sequences, wherein the Pbad promoter drives expression of Cas1 and Cas2 proteins based on the presence of arabinose.

In another embodiment, provided is a bacterial cell comprising the Pbad containing plasmid described in the preceding paragraph. In specific embodiments, the bacterial cell is KP08, EC77 or BL21.

The other improvement described herein for increasing efficiency of the CRISPR-Cas recording system involves the engineering of mutated versions of Cas-1 or Cas-2 as shown in FIG. 25. Version 2 (V2) pertains to Cas1 with a P10L, mutation and Version 3 (V3) pertains to Cas2 with an E52G mutation. FIG. 27 shows how these mutated versions increase expansion efficiency of the CRISPR array. FIG. 29 represents an experiment showing increase spacer acquisition based on oxidative stress pulsing. FIG. 30 shows that versions V2 and V3 are capable of increased spacer acquisition in multiple bacterial strains.

According to certain embodiments, provided is a nucleic acid sequence encoding Cas1 (V2) with a P10L mutation. Another embodiment pertains to a nucleic acid sequence encoding Cas2 (V3) with an E52G mutation. Related embodiments pertain to a plasmid comprising a nucleic acid sequence encoding Cas1, a nucleic acid sequence encoding Cas2 and a promoter for driving expression of Cas1 and Cas2, wherein Gas' pertains to V2 or Cas2 pertains to V3, or where Cas1 and Cas2 are V2 and V3, respectively. Other related embodiments pertain to a bacterial cell containing such plasmid. The bacterial cell may comprise EcBL, EcN, Ec257, Ef, Se, Kp08, Ko, or Pa (see FIG. 30).

The present disclosure provides for a composition comprising the present biological recording system.

### DESCRIPTION OF THE DRAWINGS

FIGS. 1A-1G show temporal recording in arrays by CRISPR expansion (TRACE). Akin to an audio tape, temporal biological signals can be stored in DNA arrays within a cell population (FIG. 1A). TRACE functions by first transforming an input biological signal to an altered abundance of a trigger DNA pool (FIG. 1B, orange). This trigger DNA pool, alongside reference DNA (FIG. 1B, blue) is then recorded as spacers into genomic CRISPR arrays of a cell population in a unidirectional fashion, enabling recording of temporal information. The pTrig trigger plasmid includes a mini-F origin for stable maintenance and an IPTG-inducible phage P1 replication system for copy number increase (FIG. 1C), qPCR measurement of pTrig relative copy number (log 10 scale) in cells exposed to no IPTG or 1 mM IPTG for 6 hours (FIG. 1D). The pRec recording plasmid includes an aTc-inducible E. coli Cas1 and Cas2 expression cassette (FIG. 1E). Experimental induction scheme and CRISPR array sequencing approach (FIG. 1F). Cells with pRec or pRec+pTrig were exposed to 100 ng/μL aTc and no or 1 mM IPTG and subjected to sequencing; resulting arrays with a single new spacer and identified source (genome, pRec or pTrig) are plotted as a percentage of all measured CRISPR arrays (FIG. 1G). Error bars represent standard deviation of three biological replicates.

FIGS. 2A-2F show temporal recording of four day input profiles. Cell populations were subjected to daily exposures over four sequential days, constituting all 16 possible temporal signal profiles (FIG. 1A). Resulting CRISPR arrays were sequenced with (black line) and without (grey dashed line) a size-enrichment method and the frequency (log 10 scale) of unexpanded (un) and expanded arrays of different lengths (L1 to maximum detectable L5) are plotted (FIG. 2B). Input profiles are grouped by number of pTrig inductions, and the percentage of pTrig spacers in each profile is displayed; red line indicates mean and standard deviation (FIG. 2C). Fifty L4 arrays sampled from the full dataset for the input profile [on, on, off, off] are shown in FIG. 2D (shaded: pTrig spacer, unshaded: reference spacer, positions p1 to p4, 5t-to-3′ of array). Spacer incorporation can be analyzed across arrays of different lengths (L) and positions (p) as a heatmap displaying percentage of pTrig spacers detected at each location. CRISPR arrays derived from recordings of all 16 temporal signal profiles (FIG. 2E). The input signal profile (left) and corresponding L4 arrays (right, shown in reverse order to improve visual comparison) are displayed in FIG. 2F.

FIGS. 3A-3F show reconstructing temporal signal profiles and population lineages. CRISPR array populations can be described as a frequency distribution constituting of all permutations of reference (R, blue) and trigger (T, orange) spacers for a given array length (L); L3 arrays are depicted in FIG. 3A. As an example, for two distinct profiles of equal number of inductions, observed (black) and model predicted (white) L3 array type frequencies are plotted (FIG. 3B); L3 positional averages are shown for reference (inset). Euclidean distance between observed data (rows) and predicted model (columns) array type distributions (L2, L3 and L4 array distributions concatenated) was calculated and normalized by row (FIG. 3C). The correct temporal signal profile is indicated by a white asterisk, and the model with minimum distance to the data is indicated by black outline on the diagonal. FIG. 3D is a graph of the number of profiles correctly classified utilizing L1 to L4 arrays individually or L2-L4 arrays together as in (FIG. 3C); grey dashed line indicates expected random classification ( 1/16). A defined branching history was utilized when performing the temporal recording experiment (FIG. 3E). The mapping locations for genomic spacers within L1 arrays was utilized as sequence identity of the spacer and the Jaccard distance between all samples (1 proportion of spacers shared between two samples) is displayed in FIG. 3F. Lineage reconstruction was performed using the Fitch-Margoliash method on this distance matrix and is displayed on the left; only one lineage is not fully differentiated (cells receiving induction on d1).

FIGS. 4A-4F show multiplex temporal recording with a barcoded sensor population. The direct repeat (DR) of a CRISPR array can be barcoded to associate sensors with specific arrays (FIG. 4A); generated distal DR barcode sequences are shown (bel ATGGTCC (SEQ ID NO: 33); bc2—ACATCAG (SEQ ID NO: 34); and WT—ATAAACC (SEQ ID NO: 37). Sensors of copper, trehalose and fucose were linked to the pTrig system and introduced into barcoded strains. The copper sensor utilizes a native promoter with endogenous transcription factor expression, while the trehalose and fucose sensors utilize an engineered transcription factor. The three barcoded sensor strains were mixed and exposed to 8 combinatorial inputs of the three chemicals; the resulting percentage of pTrig spacers for each barcoded sensor strain is displayed in FIG. 4B (average of three biological replicates). The strain mixture was exposed to combinatorial inputs over three days. As an example, profile #5 is displayed in FIG. 4C, along with CRISPR arrays for each sensor (plotted as in FIG. 2, but the color map is resealed for each sensor to aid visualization), and resulting classification (correct: blue checkmark or incorrect: red X). 16 profiles were tested (6 defined, 10 randomly generated) of 512 (8\hat{ }3) possible profiles (FIG. 4D); the resulting classification is shown as in FIG. 4C. FIG. 4E shows single channel classification accuracy: profiles were classified for each sensor using L2 and L3 arrays; grey dashed line indicates expected random classification (2/16). FIG. 4F shows multi-channel classification accuracy: predictions were considered across all three sensors, and the number classified correctly within a Hamming distance threshold is shown (black line) compared to the expected random classification (grey dashed line).

FIG. 5 shows pTrig copy number induction. To assess pTrig copy number increase in the context of recording, pTrig copy number was measured by qPCR. Cells with only pTrig displayed high copy number in the absence of inducer and low fold increase in copy number, since only genomic expression of Lad was present to repress the Lac promoter upstream of RepL on pTrig. The addition of pRec (which expresses Lad) resulted in repression of copy number in the absence of inducer and high fold increase in copy number after induction. Addition of aTc slightly decreased pTrig copy number during induction, indicating that Cas1 and Cas2 expression may reduce the apparent copy number of pTrig, for example by degradation.

FIGS. 6A-6C show decoupling pTrig copy number induction. To better understand the mechanism of pTrig copy number induction, RepL expression was decoupled from the amplifying effects of pTrig copy number increase (FIG. 6A). RepL was codon optimized for E. coli (to remove the origin of replication located within the RepL, coding sequence) and placed on a p15A plasmid (pTrig-dec-RepL). The pTrig plasmid was then modified to remove the upstream promoter and first 100 bp of the RepL coding sequence, and a terminator (L3S1P52) was placed immediately upstream (to retain the RepL oriL origin of replication but eliminate expression; pTrig-dec-oriL). The Lac promoter along with RiboJ and B0034 RBS was placed upstream of the RepL (pTrig-dec-RepL-Lac), and the decoupled system (pTrig-dec-RepL-Lac pTrig-dec-oriL+pRec) was exposed to aTc and varying concentrations of IPTG for 6 hours alongside the pTrig system utilized in the main text (FIG. 6B). Plasmid copy number of pTrig or pTrig-dec-oriL was then measured by qPCR. The decoupled system displayed reduced range in copy induction and lower sensitivity to input compared to pTrig, suggesting that a positive feedback loop may mediate induction of the pTrig system. To assess generality of the result, the experiment was repeated with a second inducible promoter (FIG. 6C). A rhamnose inducible promoter was swapped into the pTrig system (pTrig-Rha, 150 bp upstream sequence of E coli RhaB, see also FIG. 18). The same promoter with the addition of RiboJ and B0034 RBS was swapped into the RepL expression plasmid (pTrig-dec-RepL-Rha), and the decoupled system (pTrig-dec-RepL-Lac+pTrig-dec-oriL+pRec ΔLacI) was compared to the pTrig system (pTrig-Rha+pRec ΔLacI) as in (FIG. 6A with aTc and varying concentrations of rhamnose inducer for 6 hours. A similar reduced copy number induction range and lower input sensitivity in the decoupled system compared to the pTrig system was also observed.

FIGS. 7A and 7B show CRISPR spacer acquisition. CRISPR expansion, calculated as the log 10 proportion of arrays detected as expanded, was assessed over the course of a single recording round (FIG. 7A). As a control, a strain harboring no plasmids was first tested. A very low amount of expansion was detected, presumably from index swapping between samples that can occur at background levels on the Illumina sequencing platform. For one of the no plasmid samples receiving only IPTG inducer, no expanded spacers were detected, therefore this replicate not plotted. Addition of pRec increased CRISPR expansion above background levels, likely due to leaky expression of Cas1 and Cas2; addition of aTc inducer greatly increased CRISPR expansion. The addition of pTrig did not affect CRISPR expansion without copy number induction by IPTG, but overall expansion increased when IPTG was added. FIG. 7B is an alternative visualization of FIG. 1G. With the pTrig plasmid in the presence of IPTG, pTrig spacer acquisition greatly increases (other pTrig bars are not present as they are too small to be visualized on this Y-axis scale). pTrig induction did not appear to affect pRec spacer acquisition, but increased genomic spacer acquisition, indicating that pTrig copy number increase may interact with genomic replication or spacer acquisition processes. Error bars represent standard deviation of three biological replicates.

FIGS. 8A and 8B show CRISPR array sequencing. FIG. 8A is a schematic of custom CRISPR amplicon sequencing approach. FIG. 8B shows an exemplary library size distribution by Bioanalyzer HS DNA assay; the smallest product (˜166 bp) corresponds to unexpanded arrays, and products of larger sizes, ˜228, ˜288, ˜348 bp, correspond to expanded arrays of 1, 2, 3 spacers respectively. FIG. 8C is a size-enriched library size distribution determined in the same manner as FIG. 8B; expanded arrays are enriched. Contaminating high molecular weight DNA was observed (presumably plasmid or genomic DNA from the PCR template) but did not affect sequencing.

FIGS. 9A-9D shows the relationship between pTrig copy number and pTrig spacer incorporation. For the same experiment shown in FIG. 6, cells were recovered and subjected to CRISPR array sequencing. For the Lac pTrig and decoupled system, the resulting proportion of pTrig spacers is displayed in log 10 scale (FIG. 9A). In FIG. 9B, spacer incorporation was directly compared to measured pTrig or pTrig-dec-oriL copy number (shown in FIG. 6), and displayed an increasing relationship. In FIGS. 9C and 9D the same data is shown for the Rha pTrig and decoupled system.

FIGS. 10A-10E show CRISPR expansion and pTrig incorporation over a single induction round. Culture growth, pTrig copy number and spacer acquisition were tracked over the course of induction and recovery to assess response dynamics of the system. Cells received aTc induction and were exposed to no IPTG or IPTG inducer; all points display the mean and standard deviation of three biological replicates. Induction of pTrig did not appear to affect cell growth as measured by optical density compared to basal maintenance of the system (FIG. 10A). Array expansion was observed after 1 hour of induction, and a large increase in spacer acquisition was observed during recovery (FIG. 10B). pTrig copy number as measured by qPCR displayed an increase beginning 3 hours after induction (FIG. 10C). In addition, copy number increased during the recovery period only when cells had been previously induced, likely due to residual IPTG inducer in recovery media. Further dilution on the subsequent day prevents this re-activation from interfering with multi-day recordings. The percentage of pTrig spacers appeared to increase after 4 hours, consistent with pTrig copy number dynamics (FIG. 10D). The duration of induction (with aTc and IPTG) was varied between 0 to 6 hours and the recovery time was adjusted such that all samples were collected at the same time (FIG. 10E). Robust recording required the full 6 hours of induction.

FIG. 11 shows CRISPR array expansion over multiple days. Samples from intermediate states (d1, d2, d3) were sequenced in addition to d4. The percent of CRISPR arrays detected as expanded in each sample is plotted; increasing array expansion was observed over the course of the experiment.

FIGS. 12A and 12B show pTrig spacer incorporation. To visualize the information encoded within individual arrays, fifty L4 arrays sampled from two representative temporal input profiles are shown as a heatmap in FIG. 12A (as in FIG. 2D) where rows are individual arrays and columns are positions in the array (shaded: pTrig spacer, unshaded: reference spacer). The individual array information can be then visualized as positional averages as described herein (FIG. 2E). Samples from d1, d2 and d3 were additionally sequenced (FIG. 12B). The resulting % pTrig spacers detected for different array lengths (L1 to L3) at different positions (p1 to p3) is plotted as in FIG. 2E. L4 and L5 arrays are omitted as a low number were detected (intermediate samples were sequenced without the enrichment protocol).

FIG. 13 shows CRISPR array length-dependence of pTrig incorporation for model parameterization. pTrig incorporation appeared to differ across different array lengths; for example the average percentage of pTrig spacers at each position of each array length for the sample receiving inducer for four days is shown. This was presumably due to the delayed activation of pTrig compared to array expansion; the first incorporations during a recording round were less likely to contain pTrig spacers as pTrig copy number had not yet increased. Therefore, highly expanded arrays may display slightly lower levels of pTrig incorporation. Given this trend, models were individually parameterized for each array-length by empirically using the average percentage of pTrig spacers found in each array length (e.g. using the values above, see Table 5).

FIGS. 14A-14B show the observed and predicted array-type frequencies for all four day input profiles. The observed array-type frequencies from experimental data and modeled array-type frequencies are displayed for all 16 input profiles as an aggregate scatter plot, with both axes in log scale (FIG. 14A). The shading of each point indicates the number of trigger spacers the specific array-type contains. Array-types not observed in a particular sample are plotted on the X axis (e.g. log frequency=−5). Results for different array lengths are shown: L2 arrays, L3 arrays, L4 arrays. A close correspondence between the observed and predicted array-type frequencies is apparent, although a subset of low frequency array-types occur more often than predicted. It was hypothesized that the model assumption of only up to one expansion per day contributed to the discrepancy between data and model. The model was altered to allow a second expansion for singly expanded arrays at with the same probability as the first expansion but scaled by a constant value (FIG. 14B). This scaling factor (0.02402) was calculated as the proportion of doubly expanded arrays observed to singly expanded arrays observed from the same control experiment utilized to parameterize expansion rates. The same plots in FIG. 13A are shown for this two expansion model, and visually display better model recapitulation of low frequency array-types, suggesting better modeling of the CRISPR expansion process. For the sake of simplicity, the single expansion model is utilized for classification, but more nuanced models of CRISPR expansion could allow for improved reconstruction performance.

FIGS. 15A-15B show the number of arrays required for classification of temporal input profiles. For each of the 16 temporal profiles, arrays were subsampled to the minimum number detected for each array length (array lengths L1 to L4; decreasing arrays detected for increasing array lengths) and classification was performed (FIG. 15A). Lines display the mean and error bands display the 95% confidence interval of 50 iterations of subsampling and classification. In the inset, arrays are subsampled to 508 arrays (the minimum number arrays detected in any sample of any array length). These results demonstrate that only a few hundred arrays of a given length are required for reasonable classification performance. The same subsampling and classification accuracy analysis was replotted with an estimate of the total array population required (log 10 scale) in FIG. 15B, rather than the number of arrays of a given length as in FIG. 15A. Specifically, the x-axis was rescaled utilizing the average proportion of arrays of a given length (L1 to L4) observed across the 16 temporal profile samples sequenced without size enrichment (FIG. 2B, dotted lines). These results demonstrate that a population of ˜10⁵ arrays (using L3 arrays for classification) can recapitulate reasonable accuracy (˜75% or 12/16 correctly classified).

FIGS. 16A-16C shows the stability of TRACE recordings. Cell populations were subjected to 3 day temporal recording, resulting in 8 temporal profiles. The 8 populations were subsequently diluted 1:100 every 24 hours into 3 mL fresh LB media with antibiotics for a total of 8 days (˜6.6 generations per day, ˜50 total generations). Array-type frequencies at d1 and d8 were then compared for all of the 8 profiles in aggregate for array length 2 (L2) (FIG. 16A) and L3 arrays (FIG. 16B). Array types not detected in a sample are plotted on the axis (e.g. log frequency=−4). Array type frequencies appeared stable over the course of the experiment, although some low frequency array types exhibited variability likely due to population fluctuations. A strain containing an array with two expanded spacers was clonally isolated and induced with aTc with the same induction protocol utilized for recording (FIG. 16C). The strain before induction (d0) and after induction (d1) was sequenced. The percentage of extracted L2 spacer sequences within Hamming distance 2 of the actual expected sequence at d0 is displayed and was >99% at each position; other spacers likely represent sequencing errors or background levels of spacer loss. After induction, L3 arrays (e.g. arrays receiving a new spacer) were analyzed; the distal p2 and p3 positions largely contained the expected spacers with a small but measurable loss (˜1%) compared to the background rate before induction. In sum, these experiments demonstrate stability of array type frequencies and thus recorded information, and a low rate of loss of previously recorded spacers.

FIGS. 17A and 17B show 10-day temporal recording. A 10 day recording (˜150 generations, ˜15 generations per day) was performed to assess the limits of long term recording. Eight of the 1024 (2\hat{ }10) possible 10-day temporal input profiles (bottom boxes) were randomly selected and 8 corresponding lineages to these input profiles were experimentally exposed in a similar manner to the 4 day experiment, utilizing a branching lineage method (FIG. 17A). Samples were collected at each time point from d4 to d10 for sequencing; given that some of the early time point substrings were shared between samples, not all early days contained 8 distinct samples (minimum 6 samples each day). Here, input exposures are displayed as a binary string (1 indicates induction and 0 indicates no induction) for clarity. The data was then classified against models of all potential profiles and for array lengths L1-L5 (FIG. 17B). Reasonable reconstruction accuracies were obtained up to d6 (L4 arrays: 4/7 tested correct, 1/64 expected by random guessing). In addition, arrays with more spacers appeared to enable better classification of input profiles of longer duration.

FIGS. 18A-18D show screening for orthogonal TRACE sensor systems. In preliminary experiments, sensors and exposure conditions were screened to identify three sensors displaying orthogonal function. In total, functionality of multi-channel sensing was demonstrated with six distinct sensing systems. First utilized was the GalS and TreR sensor strains alongside the LacI sensor system (FIG. 18A). Each strain responded to its cognate inducer, however the GalS sensor displayed inactivation in the presence of IPTG and trehalose. Inactivation of the GalS sensor in response to IPTG has been previously reported by Shis, D. L., et al. (ACS Synth. Biol. 3, 645-651 (2014)), consistent with this result. A rhamnose sensor was constructed, consisting of the 150 bp upstream sequence of E. coli RhaB swapped in place of the Lac promoter pTrig (pTrig-Rha); no transcription factor overexpression was utilized for this sensor system (FIG. 18B). This sensor was tested alongside the GalS and TreR sensor strains containing barcoded DR sequences. Populations of cells were exposed to combinatorial inputs (1 mM rhamnose [R] was used as inducer for the Rha sensor). Again, cognate response of each sensor to its ligand was observed; however, inactivation of the Rha sensor was observed in the presence of trehalose. The experiment outlined in FIG. 18B was repeated but with 10 mM rhamnose rather than 1 mM rhamnose in an attempt to avoid trehalose inactivation of the Rha sensor strain (FIG. 18C). However, with these inducer conditions, inactivation of the GalS sensor was observed in the presence of rhamnose. These results highlight the complex interplay of endogenous sensing systems in E. coli, likely reflecting host sugar utilization hierarchies. A 3OC6-HSL (e.g. AHL) sensor was constructed by swapping the D49 promoter in place of the Lac promoter (pTrig-D49) and expressing the LuxR transcription factor on a variant of the pRec plasmid (pRec-LuxR) (FIG. 18D). Populations of cells were exposed to combinatorial inputs (100 nM 3OC6-HSL [A] was used as inducer for the LuxR sensor). Each sensor displayed a response only to its cognate input.

FIGS. 19A-19D show multi-channel recording with the TRACE system. pTrig copy number characterization by qPCR for each of the three sensing systems individually exposed to their cognate input for 6 hours (FIG. 19A). Fold increase (linear scale) in the percentage of pTrig spacers after recording for 7 input conditions compared to no inducer (FIG. 19B); all systems display >24 fold increase (each value displays the average of three biological replicates). The TreR sensor displays a higher fold increase compared to the two other systems. The frequency of each of the three barcoded CRISPR arrays after the 8 inducer input exposures is shown in FIG. 19C. All sensors are detected in each of conditions although with differing frequencies, suggesting that subtle fitness differences between sensor strains and during pTrig activation may result in altered population abundances. The percentage of expanded arrays detected for each of the three sensors is shown in FIG. 19D; the two barcoded arrays (TreR and GalS sensors) display similar expansion to the wild type array (CopA). Barcoding does not impede the CRISPR expansion process for the two barcode sequences tested.

FIGS. 20A and 20B show population frequencies and pTrig spacer incorporation for the multiplex temporal recording experiment. The final frequency of each of the three barcoded CRISPR arrays at d3 is displayed for all 16 temporal profiles tested (FIG. 20A); frequencies vary per profile and sensor but all three are detected in each sample at a frequency of at least ˜0.4%. FIG. 20B shows average pTrig spacer incorporation for different array lengths (L1 to L3) and positions (p1 to p3) plotted as in FIG. 2E. To aid visualization, the color map for the CopA sensor ranges from 0 to 8%, while the color map for the TreR and GalS sensors ranges from 0 to 30%.

FIG. 21 provides a diagram of the general concept of recording DNA transfer into clinical enterobacterial isolates.

FIG. 22 is a diagram of a two plasmid system for recording in different hosts. One plasmid (left) includes the cas1-2 genes and the other plasmid (right) includes the CRISPR array.

FIG. 23 is a plasmid diagram that includes a plasmid wherein the promoter controlling the expression of Cas1-2 in the original recording plasmid (pRec4) is substituted with a Pbad promoter. The Pbad promoter is driven by arabinose.

FIG. 24 are gel photographs illustrating that the Pbad containing plasmid generates high recording efficiency. The pRec6 plasmid shows high recording efficiency in Kp08, Ec77, and BL21.

FIGS. 25A-25C show directed evolution of E. coli Cas1-Cas2 complex for accelerating CRISPR array expansion. FIG. 25A is a schematic diagram of the directed evolution strategy. FIG. 25B is a bar graph for the activities of the engineered variants (v1, v2, v3). FIG. 25C is structural analysis (mutated positions in the crystal structure).

FIGS. 26A-26C show validation of the directed evolution strategy for Cas1-Cas2 complex. FIG. 26A shows the number of T7 promoter in the CRISPR array affects reporter gene expression. FIG. 26B shows quantification of T7 spacer harboring cells using flow cytometer and sequencing. FIG. 26C shows Kanamycin selection enriches population with expanded arrays with T7 spacers. All measurements are based on three biological replicates.

FIGS. 27A-27D show evaluation of the variants isolated from 1^{st} and 2^{nd} round screening. FIG. 27A shows pCas12-v1 vs pCas12-v1-ev variant (isolated from 1^{st} round screening) activity. FIG. 27B shows pCas12-v1 vs pCas12-v1-ev variant (isolated from 1^{st} round screening) copy number. FIG. 27C shows the resolving effect of the mutations found in pCas12-v1-ev variant. FIG. 27C shows pCas12-v2 vs pCas12-v3 (isolated from 2^{nd} round screening) activity. All measurements are based on three biological replicates.

FIG. 28A-28C show characterization of CRISPR array expansion by the engineered E. coli Cas1-Cas2 variants. FIG. 28A shows alignment of spacer source to genome. FIG. 28B is PAM motif analysis. FIG. 28C shows the proportion of spacers derived from genome or plasmid.

FIGS. 29A-B show that accelerated CRISPR array expansion enables high resolution temporal recording of signal profiles (TRACE) and population lineages. FIG. 29A is a schematic diagram of TRACE for oxidative stress (v1, v2). Different lengths of oxidative stress pulse were given to the pCas12-v1 or pCas12-v2 harboring recording cell population with pTrig-SoxS (engineered pTrig with E. coli SoxS promoter to sense oxidative stress).
Varying signal pulse length
2 hr expression, then 0, 0.5, 1, 2, 3, 4, 5, 6, 8 hr pulse with aTc for 10 hour for all
Proportion of pTrig-derived spacers among all arrays (absolute)
Proportion of pTrig-derived spacers among expanded arrays (relative)
Array type profiles—Z-score, clustermap
Principal component analysis—clustering performances
Train a model, then compare Predicted vs Actual
   FIG. 29B is a graph of performance of pCas12-v1 and pCas12-v2 to capture oxidative stress with different pulse lengths. pCas12-v2 is more efficient and more sensitive to capture and record biological signals with higher temporal resolution.
   All measurements are based on three biological replicates.

FIG. 30 shows evolved Cas-1-Cas2 variants can be ported into other strains. Provided is a graph showing that the E. coli CRISPR adaptation system can be ported into other strains (including non-lab E. coli strains, gut isolates, Salmonella, Klebsiella, and Pantoea). pCas12-v1, v2, or v3 were introduced into 8 different strains together with p15a-Array plasmid (A p15a origin-based plasmid with E. coli BL21 CRISPR array I), and their activities were compared after overnight expression of Cas1 and Cas2 with 100 ng/uL of aTc. All measurements are based on three biological replicates.

FIG. 31 is a diagram introducing how the embodiments described herein may be implemented for in vivo sensing & lineage tracing studies in murine GI tract. Lineage tracing can determine residence time, cell population, and spatiotemporal heterogeneity by sensing for foods, toxins, and inflammation markers, for example. Recording can be coupled to actuation events.

FIG. 32 is a diagram introducing how the embodiments described herein may be implemented to conduct real-time recording of horizontal gene transfer (HGT).

FIG. 33 is a diagram representing how spacer acquisition records exposure to foreign DNA on CRISPR arrays. EcRec strain uses Type I-E CRISPR-Cas (E. coli BL21 background). Inducible over-expression of Cas1/2 using pRec plasmid. No CRISPR interference machinery present.

FIG. 34 is a diagram representing direct real-time recording of HGT of conjugative plasmid into CRISPR array.

FIG. 35 is a graph showing the determination of stringent cutoffs for high confidence spacer analysis.

FIG. 36 is a graph showing nucleotide resolution mapping of HGT DNA by assessing new spacers.

FIG. 37 is a graph showing how exogenous spacer capture efficiency is dependent on abundance of donors.

FIG. 38 is a diagram showing how embodiments described herein can record transient and non-replicative HGT events.

FIG. 39 is a diagram showing how embodiments described herein can achieve multiplex delineation of mobilization potential of different plasmids.

FIG. 40 is graphs showing how though all plasmids are predicted to be “mobilizable,” only some transfer.

FIG. 41 is graphs showing how recording in a synthetic community enables HGT comparisons in a population.

FIG. 42 is diagrams showing how embodiments described herein can test natural samples to determine if HGT can be measured in native settings.

### DETAILED DESCRIPTION

Provided herein are methods and systems to record temporal biological signals into the genomes of engineered cells (e.g., genomes of a bacterial population) using the CRISPR-Cas system. This “biological tape recorder” technology can robustly and accurately profile time-varying biological signals and regulatory programs. In certain embodiments, biological signals trigger intracellular DNA production that is then recorded by the CRISPR-Cas system. This approach enables stable recording over a desired period of time (e.g., multiple days, weeks, months, or even longer), and accurate reconstruction of temporal and lineage information by sequencing CRISPR arrays. Moreover, a multiplexing strategy can be used to simultaneously record a plurality of biological signals over time. The present method and system enable the temporal measurement of dynamic cellular states and environmental changes.

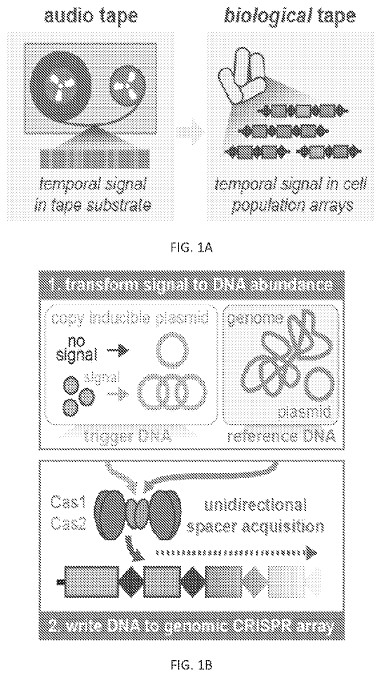

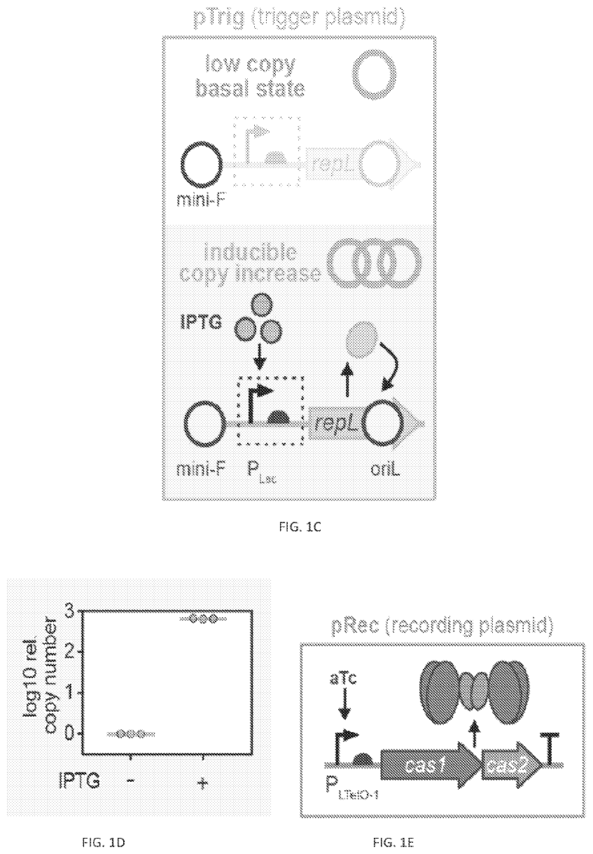

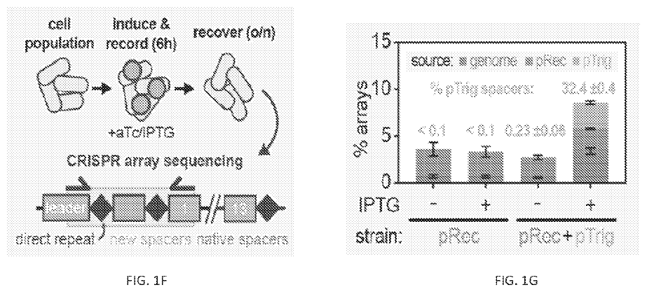

In certain embodiments, the present method and system is temporal recording in arrays by CRISPR expansion (TRACE). In this framework, a biological input signal is first transformed into a change in the abundance of a trigger DNA pool within living cells. The CRISPR-Cas spacer acquisition machinery is then employed to record the amount of trigger DNA into CRISPR arrays in a unidirectional manner (FIG. 1B). Through this architecture, the presence of an input signal increases the frequency of trigger spacers incorporated into arrays, which constitutes recording of the positive signal. However, in the absence of a signal, reference spacers can still be acquired into arrays at a background rate from sources other than the trigger DNA, such as the genome. These reference spacers serve as pace-denoting markers that are embedded during the recording session, akin to the physical spacing on a tape substrate that represents time intervals.

The present method and system may be utilized to record metabolite fluctuations, gene expression changes, and lineage-associated information across cell populations in difficult-to-study habitats such as the mammalian gut or in open settings such as soil or marine environments. The system could employ inducible intracellular DNA production systems in parallel (See, for example, J. Elbaz, P. Yin, C. A. Voigt, Nature Communications. 7, 11179 (2016), incorporated herein by reference in its entirety) and other CRISPR-Cas adaptation machinery (See for example, S. A. Jackson et al., Science. 356, eaa15056 (2017) and S. Silas et al., Science. 351, aad4234 (2016), each incorporated herein by reference in their entirety), which may be needed for extension to other bacteria (or eukaryotes) and to increase the temporal resolution of recording. The system could be further modified by increasing the spacer incorporation rate (See for example, R. Heler et al., Molecular Cell. 65, 168-175 (2017), incorporated herein by reference in its entirety), increasing the sequencing length (e.g. by nanopore sequencing), and improving reconstruction algorithms. These advances could further facilitate biological recording of inputs across many signal channels, with higher temporal resolution, and in smaller populations, e.g., down to single cells. TRACE should greatly advance the ability to delineate and understand complex cellular processes across time.

The present disclosure provides for a method of recording a temporal biological signal in an engineered, non-naturally occurring cell, comprising: exposing the cell to a temporal biological signal, wherein the cell comprises a trigger nucleic acid and a CRISPR-Cas system, wherein the CRISPR-Cas system comprises an CRISPR array nucleic acid sequence, wherein the trigger nucleic acid comprises at least one oligonucleotide spacer, wherein presence and/or strength of the temporal biological signal correlates with an abundance of the oligonucleotide spacer, wherein the CRISPR-Cas system unidirectionally inserts the oligonucleotide spacer into the CRISPR array nucleic acid sequence, and wherein the abundance of the oligonucleotide spacer correlates with a frequency of the oligonucleotide spacer inserted into the CRISPR array nucleic acid sequence.

The present disclosure provides for a method of recording a plurality of temporal biological signals in engineered, non-naturally occurring cells, comprising: (a) mixing a plurality of populations of cells to generate mixed cells, each population of cells comprising a trigger nucleic acid and a CRISPR-Cas system, wherein the CRISPR-Cas system comprises an CRISPR array nucleic acid sequence, wherein the trigger nucleic acid comprises one or more oligonucleotide spacers, wherein the oligonucleotide spacers in different populations of cells differ; and (b) exposing the mixed cells to a plurality of temporal biological signals, wherein presence and/or strength of each temporal biological signal correlates with an abundance of a corresponding oligonucleotide spacer, and wherein the CRISPR-Cas system unidirectionally inserts the oligonucleotide spacer into the CRISPR array nucleic acid sequence, wherein the abundances of the oligonucleotide spacers correlate with frequencies of the oligonucleotide spacers inserted into the CRISPR array nucleic acid sequence.

In certain embodiments, the oligonucleotide spacers are barcoded. In one embodiment, the oligonucleotide spacers are barcoded via a nucleic acid sequence of a direct repeat (DR) sequence of the CRISPR array nucleic acid sequence.

Also encompassed by the present disclosure is a biological recording system comprising: an engineered, non-naturally occurring cell comprising a trigger nucleic acid and a CRISPR-Cas system, wherein the CRISPR-Cas system comprises an CRISPR array nucleic acid sequence, wherein the trigger nucleic acid comprises at least one oligonucleotide spacer, wherein an abundance of the oligonucleotide spacer is increased by presence and/or strength of a temporal biological signal, wherein the CRISPR-Cas system unidirectionally inserts the oligonucleotide spacer into the CRISPR array nucleic acid sequence, and wherein the abundance of the oligonucleotide spacer correlates with a frequency of the oligonucleotide spacer inserted into the CRISPR array nucleic acid sequence.

In one embodiment, the CRISPR-Cas system additionally inserts one or more reference spacers into the CRISPR array nucleic acid sequence. For example, the reference spacers may be derived from the cell's genome and/or one or more plasmids in the cell.

In certain embodiments, the TRACE methods described herein utilize the E. coli CRISPR-Cas machinery as a high-performance memory device that links biological inputs to altered patterns of CRISPR spacer acquisition.

As used herein, the term “trigger nucleic acid” refers to a nucleic acid the abundance of which correlates with a biological signal. In certain embodiments, a copy number of the trigger nucleic acid is increased by presence and/or strength of a temporal biological signal. In certain embodiments, the trigger nucleic acid is a plasmid. In certain embodiments, the trigger nucleic acid comprises at least one oligonucleotide spacer which can be inserted into a CRISPR array nucleic acid sequence by a CRISPR-Cas system.

The engineered, non-naturally occurring cell may be a prokaryotic cell or a eukaryotic cell. In certain embodiments, the prokaryotic cell is a bacterial cell, such as Escherichia coli. In certain embodiments, the eukaryotic cell is a yeast cell, plant cell or a mammalian cell (e.g., a human cell).

In certain embodiments, the replication of the trigger nucleic acid is directly or indirectly affected (e.g., increased) by a biological signal. In the presence of a biological signal, or when the strength of the biological signal increases, a regulatory element either resides outside of the trigger nucleic acid, or as part of the trigger nucleic acid, directly or indirectly increases the replication of the trigger nucleic acid, thus increasing the copy number of the trigger nucleic acid, and an abundance of the oligonucleotide spacer. In other words, the regulatory element may act as a sensor for the biological signal.

The present method and system may contain a plurality of different sensors (e.g., the regulatory element) for multiplex sensing. The sensor may be naturally occurring or may be synthetic. Non-limiting examples of the sensors include natural promoters, non-natural promoters, a transcription factor that can be overexpressed, and E. coli metal responsive promoters. In certain embodiments, the sensor is a natural or modified promoter from E. coli.

In certain embodiments, the sensor is an engineered sensing system of biomarkers of disease states such as inflammation (e.g., Thiosulfate and Tetrathionate). See for example, Daeffler et al., Mol. Sys. Bio., 2017, 13(4): 923 and Riglar et al., Nature Biotechnology 35, 653-658 (2017), each incorporated herein by reference in their entirety. In certain embodiments, the sensor is a promoter from libraries of E. coli genomic promoters and can sense complex transcriptional profiles that may be associated with specific disease conditions.

Any suitable sensors that can link the presence (or absence), and/or strength, of a biological signal with a responsive element (e.g., a trigger nucleic acid such as the pTrig plasmid as discussed in Example 1) can be used. In certain embodiments, the sensor is a genomic promoter. In certain embodiments, the sensor/signal system is LacI/IPTG, GalS/fucose, TreR/trehalose, LuxR/AHL, CopA/copper, and Rha/rhamnose as described herein.

The present method and system may contain any suitable Cas systems for various modifications/improvements in recording and/or transfer to other systems. In certain embodiments, the Cas enzyme is Cas1 and/or Cas2 which is conserved across many CRISPR systems in bacteria and archaea. In certain embodiments, the Cas enzyme is a Cas1 homologue (or Cas2 homologue) which may confer various recording properties. In one embodiment, RT-Cas1 is used for RNA recording utilizing RT-Cas1 (See, for example, S. Silas et al., Science. 351, aad4234 (2016), incorporated herein by reference in its entirety). In certain embodiments, different Cas1/2 systems having different inherent efficiencies are used to confer different recording rates. In certain embodiments, other Cas1/2 systems are used to port the system to different bacteria or archaea.

The trigger nucleic acid of the present method and system may be any suitable intracellular DNA production modalities. In certain embodiments, multiple independent plasmid-copy number systems could be utilized to record different signals simultaneously. In certain embodiments, DNA-production modalities such as reverse transcriptases could be utilized to produce a dsDNA hairpin from an RNA substrate (See, for example, J. Elbaz, P. Yin, C. A. Voigt, Nature Communications. 7, 11179 (2016), incorporated herein by reference in its entirety). In certain embodiments, different trigger nucleic acids may confer different recording properties, e.g. different dynamic responses to input signals.

The present method and system may be used in various environmental settings for sentinel/surveillance applications. In certain embodiments, the temporal availability of heavy metals (such as copper) can be recorded. In certain embodiments, the temporal availability of other environmental contaminants and/or and pollutants, such as arsenic, zinc, iron etc. is recorded. In certain embodiments, the amounts of explosives or chemical warfare agents in an environment are recorded.

The present method and system may be deployed into in vivo settings (e.g., mammalian gut) for diagnostic applications. In certain embodiments, the temporal availability of one or more sugars (e.g., fucose) can be recorded. In one embodiment, the fucose concentration is associated with infection in a mammal. In certain embodiments, the spatial profiles of signals (linked to temporal transit of bacteria across the gut, e.g. small intestine vs. large intestine) are recorded.

The present method and system may be deployed for population-wide sensing applications. In one embodiment, the recording system is barcoded, and each individual is administered a population of cells (e.g., bacterial cells) with a unique barcode among the different individuals. The populations of cells may be recovered from a mixed location (e.g., sewage) and the barcode can be utilized to associate specific signals to specific individuals.

The present method and system may be deployed as a fingerprinting device. In certain embodiments, individual spacers are unique and populations of arrays can be utilized to trace population and lineage history. In certain embodiments, the system could be utilized for authentication applications, for example to ensure that a specific bacterial strain or population was derived from another specific strain or population. In certain embodiments, the system could be utilized for fingerprinting or tracking purposes, for example to track the surfaces an individual has touched and to estimate the time points when the surfaces were touched. In certain embodiments, the system could be utilized for tracking purposes, for example to track the surfaces an object (e.g., vehicles, ships, packages, etc.) has touched and to estimate the time points when the surfaces were touched.

The present methods and systems may be used for signal reconstruction, population history reconstruction, etc. The present methods and systems have various applications such as forensics applications, authentication applications, determining provenance of a bacterial strain of interest, etc. In certain embodiments, the sequence identity of inserted spacers (e.g., references spacers derived from the cell's genome and/or one or more plasmids in the cell) can be analyzed to reconstruct population history and lineage of a complex cell population, in addition to signal reconstruction. In one embodiment, the present disclosure provides for a method and system to reconstruct lineage information for tracking or forensic applications, e.g., using the reference spacer information. In one embodiment, the information provided by the reference spacers, and the information provided by the oligonucleotide spacers (e.g., derived from the trigger nucleic acid) provide two layers of recorded information.

In certain embodiments, the present disclosure provides for a method for reconstructing complex population histories and/or cell lineages, comprising the step of analyzing the sequence identity of incorporated CRISPR spacers (e.g., reference spacers, and/or oligonucleotide spacers, e.g., derived from the trigger nucleic acid). The incorporated spacers contain unique nucleotide sequence information, and within arrays the ordering of different spacers encodes additional information, constituting a continuously generated unique barcode in cells.

The present disclosure provides for a method of reconstructing lineage of cells, comprising: analyzing a sequence identity of a plurality of reference spacers inserted into a CRISPR array nucleic acid sequence in the cells, wherein the cells comprise a CRISPR-Cas system comprising the CRISPR array nucleic acid sequence. For example, the reference spacers may be derived from the cell's genome and/or one or more plasmids in the cell.

As used herein, the term “strength” may refer to amplitude, frequency, incidence, etc. of, e.g., a signal.

### Molecular Biology

In accordance with the present invention, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

As used herein and in the claims, the singular forms “a,” “an,” and “the” include the singular and the plural reference unless the context clearly indicates otherwise. Thus, for example, a reference to “an agent” includes a single agent and a plurality of such agents.

“Treating” or “treatment” of a state, disorder or condition includes: (1) preventing or delaying the appearance of clinical symptoms of the state, disorder, or condition developing in a person who may be afflicted with or predisposed to the state, disorder or condition but does not yet experience or display clinical symptoms of the state, disorder or condition; or (2) inhibiting the state, disorder or condition, e.g., arresting, reducing or delaying the development of the disease or a relapse thereof (in case of maintenance treatment) or at least one clinical symptom, sign, or test, thereof; or (3) relieving the disease, e.g., causing regression of the state, disorder or condition or at least one of its clinical or sub-clinical symptoms or signs. The benefit to a subject to be treated is either statistically significant or at least perceptible to the patient or to the physician.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

An “immune response” refers to the development in the host of a cellular and/or antibody-mediated immune response to a composition or vaccine of interest. Such a response usually consists of the subject producing antibodies, B cells, helper T cells, suppressor T cells, regulatory T cells, and/or cytotoxic T cells directed specifically to an antigen or antigens included in the composition or vaccine of interest.

A “therapeutically effective amount” means the amount of a compound that, when administered to an animal for treating a state, disorder or condition, is sufficient to affect such treatment. The “therapeutically effective amount” will vary depending on the compound, the disease and its severity and the age, weight, physical condition and responsiveness of the animal to be treated.

The compositions of the invention may include a “therapeutically effective amount” or a “prophylactically effective amount” of a compound described herein. A “therapeutically effective amount” refers to an amount effective, at dosages and for periods of time necessary, to achieve the desired therapeutic result. A therapeutically effective amount of an antibody or antibody portion may vary according to factors such as the disease state, age, sex, and weight of the individual, and the ability of the antibody or antibody portion to elicit a desired response in the individual. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

While it is possible to use a composition provided by the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. Accordingly, in one aspect, the present invention provides a pharmaceutical composition or formulation comprising at least one active composition, or a pharmaceutically acceptable derivative thereof, in association with a pharmaceutically acceptable excipient, diluent and/or carrier. The excipient, diluent and/or carrier must be “acceptable” in the sense of being compatible with the other ingredients of the formulation and not deleterious to the recipient thereof.

The compositions of the invention can be formulated for administration in any convenient way for use in human or veterinary medicine. The invention therefore includes within its scope pharmaceutical compositions comprising a product of the present invention that is adapted for use in human or veterinary medicine.

In a preferred embodiment, the pharmaceutical composition is conveniently administered as an oral formulation. Oral dosage forms are well known in the art and include tablets, caplets, gelcaps, capsules, and medical foods. Tablets, for example, can be made by well-known compression techniques using wet, dry, or fluidized bed granulation methods.

Such oral formulations may be presented for use in a conventional manner with the aid of one or more suitable excipients, diluents, and carriers. Pharmaceutically acceptable excipients assist or make possible the formation of a dosage form for a bioactive material and include diluents, binding agents, lubricants, glidants, disintegrants, coloring agents, and other ingredients. Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used. An excipient is pharmaceutically acceptable if, in addition to performing its desired function, it is non-toxic, well tolerated upon ingestion, and does not interfere with absorption of bioactive materials.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

“Patient” or “subject” refers to mammals and includes human and veterinary subjects.

The dosage of the therapeutic formulation will vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. In some cases, oral administration will require a higher dose than if administered intravenously. In some cases, topical administration will include application several times a day, as needed, for a number of days or weeks in order to provide an effective topical dose.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, olive oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “subject” as used in this application means an animal with an immune system such as avians and mammals. Mammals include canines, felines, rodents, bovine, equines, porcines, ovines, and primates. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject.

The terms “screen” and “screening” and the like as used herein means to test a subject or patient to determine if they have a particular illness or disease, or a particular manifestation of an illness or disease. The term also means to test an agent to determine if it has a particular action or efficacy.

The terms “identification”, “identify”, “identifying” and the like as used herein means to recognize a disease state or a clinical manifestation or severity of a disease state in a subject or patient. The term also is used in relation to test agents and their ability to have a particular action or efficacy.

The terms “prediction”, “predict”, “predicting” and the like as used herein means to tell in advance based upon special knowledge.

The term “reference value” as used herein means an amount or a quantity of a particular protein or nucleic acid in a sample from a healthy control or healthy donor.

The terms “healthy control”, “healthy donor” and “HD” are used interchangeably in this application and are a human subject who is not suffering from a disease or a condition.

The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “agent” as used herein means a substance that produces or is capable of producing an effect and would include, but is not limited to, chemicals, pharmaceuticals, biologics, small organic molecules, antibodies, nucleic acids, peptides, and proteins.

The phrase “therapeutically effective amount” is used herein to mean an amount sufficient to cause an improvement in a clinically significant condition in the subject, or delays or minimizes or mitigates one or more symptoms associated with the disease, or results in a desired beneficial change of physiology in the subject.

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

The term “purified” and the like as used herein refers to material that has been isolated under conditions that reduce or eliminate unrelated materials, e.g., contaminants. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

The terms “expression profile” or “gene expression profile” refers to any description or measurement of one or more of the genes that are expressed by a cell, tissue, or organism under or in response to a particular condition. Expression profiles can identify genes that are up-regulated, down-regulated, or unaffected under particular conditions. Gene expression can be detected at the nucleic acid level or at the protein level. The expression profiling at the nucleic acid level can be accomplished using any available technology to measure gene transcript levels.

For example, the method could employ in situ hybridization, Northern hybridization or hybridization to a nucleic acid microarray, such as an oligonucleotide microarray, or a cDNA microarray. Alternatively, the method could employ reverse transcriptase-polymerase chain reaction (RT-PCR) such as fluorescent dye-based quantitative real time PCR (TaqMan® PCR). In the Examples section provided below, nucleic acid expression profiles were obtained using Affymetrix GeneChip® oligonucleotide microarrays. The expression profiling at the protein level can be accomplished using any available technology to measure protein levels, e.g., using peptide-specific capture agent arrays.

The terms “gene”, “gene transcript”, and “transcript” are used somewhat interchangeably in the application. The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. “Transcript” or “gene transcript” is a sequence of RNA produced by transcription of a particular gene. Thus, the expression of the gene can be measured via the transcript.

The term “antisense DNA” is the non-coding strand complementary to the coding strand in double-stranded DNA.

The term “genomic DNA” as used herein means all DNA from a subject including coding and non-coding DNA, and DNA contained in introns and exons.

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (e.g., will not prevent formation of an anti-parallel hybrid).

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include, but are not limited to, plasmids, phages, and viruses.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA which codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct” or “gene construct.” A common type of vector is a “plasmid”, which generally is a self-contained molecule of double-stranded DNA, usually of bacterial origin, that can readily accept additional (foreign) DNA and which can be readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “host cell” means any cell of any organism that is selected, modified, transformed, grown, used or manipulated in any way, for the production of a substance by the cell, for example, the expression by the cell of a gene, a DNA or RNA sequence, a protein or an enzyme. Host cells can further be used for screening or other assays, as described herein.

A “polynucleotide” or “nucleotide sequence” is a series of nucleotide bases (also called “nucleotides”) in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide. This includes single- and double-stranded molecules, e.g., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil.

“Nucleic acid” refers to deoxyribonucleotides or ribonucleotides and polymers thereof in either single- or double-stranded form. The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, and carbamates) and with charged linkages (e.g., phosphorothioates, and phosphorodithioates). Polynucleotides may contain one or more additional covalently linked moieties, such as, for example, proteins (e.g., nucleases, toxins, antibodies, signal peptides, and poly-L-lysine), intercalators (e.g., acridine, and psoralen), chelators (e.g., metals, radioactive metals, iron, and oxidative metals), and alkylators. The polynucleotides may be derivatized by formation of a methyl or ethyl phosphotriester or an alkyl phosphoramidate linkage. Modifications of the ribose-phosphate backbone may be done to facilitate the addition of labels, or to increase the stability and half-life of such molecules in physiological environments. Nucleic acid analogs can find use in the methods of the invention as well as mixtures of naturally occurring nucleic acids and analogs. Furthermore, the polynucleotides herein may also be modified with a label capable of providing a detectable signal, either directly or indirectly. Exemplary labels include radioisotopes, fluorescent molecules, and biotin.

The term “polypeptide” as used herein means a compound of two or more amino acids linked by a peptide bond. “Polypeptide” is used herein interchangeably with the term “protein.”

The term “about” or “approximately” means within an acceptable error range for the particular value as determined by one of ordinary skill in the art, which will depend in part on how the value is measured or determined, e.g., the limitations of the measurement system, e.g., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed. CRISPR

In certain embodiments, the Cas enzyme is Cas1, Cas2, Cas1B, Cas9, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, orthologs thereof, or modified versions thereof. In one embodiment, the Cas enzyme is Cas1 and/or Cas2.

In certain embodiments, the Cas enzyme comprises one or more mutations. In a specific embodiment, the Cas enzyme pertains to Cas1 (V2), Cas1 with a P10L mutation, or Cas2 (V3), Cas2 with an E52G mutation.

In certain embodiments, the Cas enzyme is codon-optimized for expression in a eukaryotic cell, such as a mammalian cell, or a human cell.

The Cas enzyme can be introduced into a cell in the form of a DNA, mRNA or protein. The Cas enzyme may be engineered, chimeric, or isolated from an organism.

Cas1 or Cas2 used in the methods and systems described herein can be any Cas1 or Cas2 present in a prokaryote. In certain embodiments, Cas1 or Cas2 is a Cas1 or Cas2 polypeptide of an archaeal microorganism. In certain embodiments, Cas1 or Cas2 is a Cas1 or Cas2 polypeptide of a Euryarchaeota microorganism. In certain embodiments, Cas1 or Cas2 is a Cas1 or Cas2 polypeptide of a Crenarchaeota microorganism. In certain embodiments, Cas1 or Cas2 is a Cas1 or Cas2 polypeptide of a bacterium. In certain embodiments, Cas1 or Cas2 is a Cas1 or Cas2 polypeptide of a gram negative or gram positive bacteria. In certain embodiments, Cas1 or Cas2 is a Cas1 or Cas2 polypeptide of Pseudomonas aeruginosa. In certain embodiments, Cas1 or Cas2 is a Cas1 or Cas2 polypeptide of Aquifex aeolicus.

In certain embodiments, Cas1 or Cas2 may be a “functional derivative” of a naturally occurring Cas1 or Cas2 protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide.

“Cas1” encompasses a full-length Cas1 polypeptide, an enzymatically active fragment of a Cas1 polypeptide, and enzymatically active derivatives of a Cas1 polypeptide or fragment thereof. Suitable derivatives of a Cas1 polypeptide or a fragment thereof include but are not limited to mutants, fusions, covalent modifications of Cas1 protein or a fragment thereof.

“Cas2” encompasses a full-length Cas2 polypeptide, an enzymatically active fragment of a Cas2 polypeptide, and enzymatically active derivatives of a Cas2 polypeptide or fragment thereof. Suitable derivatives of a Cas2 polypeptide or a fragment thereof include but are not limited to mutants, fusions, covalent modifications of Cas2 protein or a fragment thereof.

In some embodiments, Cas1 is encoded by a nucleotide sequence provided in GenBank as, e.g., GeneID numbers: 2781520, 1006874, 9001811, 947228, 3169280, 2650014, 1175302, 3993120, 4380485, 906625, 3165126, 905808, 1454460, 1445886, 1485099, 4274010, 888506, 3169526, 997745, 897836, or 1193018. In certain embodiments, Cas 1 is encoded by a nucleotide sequence provided in GenBank as GeneID number 947228 (E. coli Cas1). In one embodiment, Cas 1 comprises the SEQ ID NO:1. The 10^{th} residue, P (bolded), of SEQ ID NO: 1 is mutated to L in version 2 (pCas12-v2) for SEQ ID NO: 35.

SEQ ID NO: 1
MTWLPLNPIPLKDRVSMIFLQYGQIDVIDGAFVLIDKTGIRTHIPVGSVA
CIMLEPGTRVSHAAVRLAAQVGTLLVWVGEAGVRVYASGQPGGARSDKLL
YQAKLALDEDLRLKVVRKMFELRFGEPAPARRSVEQLRGIEGSRVRATYA
LLAKQYGVTWNGRRYDPKDWEKGDTINQCISAATSCLYGVTEAAILAAGY
APAIGFVHTGKPLSFVYDIADIIKFDTVVPKAFEIARRNPGEPDREVRLA
CRDIIRSSKTIAKLIPLIEDVLAAGEIQPPAPPEDAQPVAIPLPVSIGDA
GHRSS*
SEQ ID NO: 35
MTWLPLNPILLKDRVSMIILQYGQIDVTDGAFVLIDKTGIRTHIPVGSVA
CIMIEPGTRVSHAAVRLAAQVGTLLVWVGEAGVRVYASGQPGGARSDKLL
YQAKLALDEDLRLKVVRKMFELRFGEPAPARRSVEQLRGIEGSRVRATYA
LLAKQYGVTWNGRRYDPKDWEKGDTINQCISAATSCLYGVTEAAILAAGY
APAIGFVHTGKPLSFVYDIADIIKFDTVVPKAFEIARRNPGEPDREVRLA
CRDIFRSSKTLAKLIPLIEDVLAAGEIQPPAPPEDAQPVAIPLPVSLGDA
GHRSS*

In certain embodiments, Cas 2 is encoded by a nucleotide sequence provided in GenBank as GeneID number 947213 (E. coli Cas2). In one embodiment, Cas 2 comprises SEQ ID NO:2. The 52^{nd} residue, E (bolded), of SEQ ID NO: 2 is mutated to G in version 3 (pCas12-v3) in SEQ ID NO: 36.

SEQ ID NO: 2
MSMLVVVTENVPPRLRGRLAIWLLEVRAGVYVGDVSAKIREMIWEQIAGL
AEEGNVVMAWATNTETGFEFQTFGLNRRTPVDLDGLRLVSFLPV*
SEQ ID NO: 36
MSMLVVVTENVPPRLRGRLAIWLLEVRAGVYVGDVSAKIREMIWEQIAGL
AGEGNVVMAWATNTETGFEFQTFGLNRRTPVDLDGLRLVSFLPV*

The term “engineered,” as used herein refers to a protein molecule, a nucleic acid, a complex, a substance, a cell, or an entity that has been designed, produced, prepared, synthesized, and/or manufactured by a human. Accordingly, an engineered product is a product that does not occur in nature.

The term “homologous,” as used herein is an art-understood term that refers to nucleic acids or polypeptides that are highly related at the level of nucleotide and/or amino acid sequence. Nucleic acids or polypeptides that are homologous to each other are termed “homologues.” Homology between two sequences can be determined by sequence alignment methods known to those of skill in the art. In accordance with the invention, two sequences are considered to be homologous if they are at least about 50-60% identical, e.g., share identical residues (e.g., amino acid residues) in at least about 50-60% of all residues comprised in one or the other sequence, at least about 70% identical, at least about 80% identical, at least about 90% identical, at least about 95% identical, at least about 98% identical, at least about 99% identical, at least about 99.5% identical, or at least about 99.9% identical, for at least one stretch of at least 20, at least 30, at least 40, at least 50, at least 60, at least 70, at least 80, at least 90, at least 100, at least 120, at least 150, or at least 200 amino acids.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The term “nuclease,” as used herein, refers to an agent, for example, a protein, capable of cleaving a phosphodiester bond connecting two nucleotide residues in a nucleic acid molecule. In some embodiments, “nuclease” refers to a protein having an inactive DNA cleavage domain, such that the nuclease is incapable of cleaving a phosphodiester bond. In some embodiments, a nuclease is a protein, e.g., an enzyme that can bind a nucleic acid molecule and cleave a phosphodiester bond connecting nucleotide residues within the nucleic acid molecule. A nuclease may be an endonuclease, cleaving a phosphodiester bonds within a polynucleotide chain, or an exonuclease, cleaving a phosphodiester bond at the end of the polynucleotide chain. In some embodiments, a nuclease is a site-specific nuclease, binding and/or cleaving a specific phosphodiester bond within a specific nucleotide sequence, which is also referred to herein as the “recognition sequence,” the “nuclease target site,” or the “target site.” In some embodiments, a nuclease is a RNA-guided (e.g., RNA-programmable) nuclease, which is associated with (e.g., binds to) an RNA (e.g., a guide RNA, “gRNA”) having a sequence that complements a target site, thereby providing the sequence specificity of the nuclease. In some embodiments, a nuclease recognizes a single stranded target site, while in other embodiments, a nuclease recognizes a double-stranded target site, for example, a double-stranded DNA target site. The target sites of many naturally occurring nucleases, for example, many naturally occurring DNA restriction nucleases, are well known to those of skill in the art. In many cases, a DNA nuclease, such as EcoRI, HindIII, or BamHI, recognize a palindromic, double-stranded DNA target site of 4 to 10 base pairs in length, and cut each of the two DNA strands at a specific position within the target site. Some endonucleases cut a double-stranded nucleic acid target site symmetrically, e.g., cutting both strands at the same position so that the ends comprise base-paired nucleotides, also referred to herein as blunt ends. Other endonucleases cut a double-stranded nucleic acid target sites asymmetrically, e.g., cutting each strand at a different position so that the ends comprise unpaired nucleotides. Unpaired nucleotides at the end of a double-stranded DNA molecule are also referred to as “overhangs,” e.g., as “5′-overhang” or as “3′-overhang,” depending on whether the unpaired nucleotide(s) form(s) the 5′ or the 5′ end of the respective DNA strand. Double-stranded DNA molecule ends ending with unpaired nucleotide(s) are also referred to as sticky ends, as they can “stick to” other double-stranded DNA molecule ends comprising complementary unpaired nucleotide(s). A nuclease protein typically comprises a “binding domain” that mediates the interaction of the protein with the nucleic acid substrate, and also, in some cases, specifically binds to a target site, and a “cleavage domain” that catalyzes the cleavage of the phosphodiester bond within the nucleic acid backbone. In some embodiments a nuclease protein can bind and cleave a nucleic acid molecule in a monomeric form, while, in other embodiments, a nuclease protein has to dimerize or multimerize in order to cleave a target nucleic acid molecule. Binding domains and cleavage domains of naturally occurring nucleases, as well as modular binding domains and cleavage domains that can be fused to create nucleases binding specific target sites, are well known to those of skill in the art.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, gRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “pharmaceutical composition,” as used herein, refers to a composition that can be administrated to a subject in the context of treatment and/or prevention of a disease or disorder. In some embodiments, a pharmaceutical composition comprises an active ingredient, e.g., a transposase fused to a Cas9 protein, or fragment thereof (or a nucleic acid encoding such a fusion), and optionally a pharmaceutically acceptable excipient. In some embodiments, a pharmaceutical composition comprises inventive Cas9 variant/fusion (e.g., fCas9) protein(s) and gRNA(s) suitable for targeting the Cas9 variant/fusion protein(s) to a target nucleic acid. In some embodiments, the target nucleic acid is a gene. In some embodiments, the target nucleic acid is an associated with a pathologic bacterial condition, whereby the allele is mutated by the action of the Cas9 variant/fusion protein(s).

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

The term “vector” refers to a polynucleotide comprising one or more recombinant polynucleotides of the present invention. Vectors include, but are not limited to, plasmids, viral vectors, cosmids, artificial chromosomes, and phagemids. The vector is able to replicate in a host cell and is further characterized by one or more endonuclease restriction sites at which the vector may be cut and into which a desired nucleic acid sequence may be inserted. Vectors may contain one or more marker sequences suitable for use in the identification and/or selection of cells which have or have not been transformed or genomically modified with the vector. Markers include, for example, genes encoding proteins which increase or decrease either resistance or sensitivity to antibiotics (e.g., kanamycin, ampicillin) or other compounds, genes which encode enzymes whose activities are detectable by standard assays known in the art (e.g., β-galactosidase, alkaline phosphatase, or luciferase), and genes which visibly affect the phenotype of transformed or transfected cells, hosts, colonies, or plaques. Any vector suitable for the transformation of a host cell (e.g., E coli, mammalian cells such as CHO cell, insect cells, etc.) as embraced by the present invention, for example, vectors belonging to the pUC series, pGEM series, pET series, pBAD series, pTET series, or pGEX series. In some embodiments, the vector is suitable for transforming a host cell for recombinant protein production. Methods for selecting and engineering vectors and host cells for expressing proteins (e.g., those provided herein), transforming cells, and expressing/purifying recombinant proteins are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

### Polynucleotides, Vectors, Cells, Kits

Also encompassed by the present disclosure is a biological recording system comprising: an engineered, non-naturally occurring cell comprising a trigger nucleic acid and a CRISPR-Cas system, wherein the CRISPR-Cas system comprises an CRISPR array nucleic acid sequence, wherein the trigger nucleic acid comprises at least one oligonucleotide spacer, wherein an abundance of the oligonucleotide spacer is increased by presence and/or strength of a temporal biological signal, wherein the CRISPR-Cas system unidirectionally inserts the oligonucleotide spacer into the CRISPR array nucleic acid sequence, and wherein the abundance of the oligonucleotide spacer correlates with a frequency of the oligonucleotide spacer inserted into the CRISPR array nucleic acid sequence.

The present disclosure provides for a kit comprising the present biological recording system, and optionally instructions for using the system.

The present disclosure provides for a composition comprising the present biological recording system.

In another embodiment of this disclosure, polynucleotides encoding one or more of the inventive proteins are provided. For example, polynucleotides encoding any of the proteins described herein are provided.

In some embodiments, vectors encoding any of the proteins described herein are provided, e.g., for recombinant expression and purification of proteins, and/or fusions comprising proteins (e.g., variants). In some embodiments, the vector comprises or is engineered to include an isolated polynucleotide, e.g., those described herein. Typically, the vector comprises a sequence encoding an inventive protein operably linked to a promoter, such that the fusion protein is expressed in a host cell.

In some embodiments, cells are provided, e.g., for recombinant expression and purification of any of the Cas enzymes provided herein. The cells include any cell suitable for recombinant protein expression, for example, cells comprising a genetic construct expressing or capable of expressing an inventive protein (e.g., cells that have been transformed with one or more vectors described herein, or cells having genomic modifications, for example, those that express a protein provided herein from an allele that has been incorporated in the cell's genome). Methods for transforming cells, genetically modifying cells, and expressing genes and proteins in such cells are well known in the art, and include those provided by, for example, Green and Sambrook, Molecular Cloning: A Laboratory Manual (4th ed., Cold Spring Harbor Laboratory Press, Cold Spring Harbor, N.Y. (2012)) and Friedman and Rossi, Gene Transfer: Delivery and Expression of DNA and RNA, A Laboratory Manual (1st ed., Cold Spring Harbor Laboratory Press, Cold Spring Harbor, N.Y. (2006)).

As used herein, the term “bacteria” encompasses both prokaryotic organisms and archaea present in mammalian microbiota.

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### EXAMPLES

### Example 1

A scalable strategy was developed to record temporal biological signals into genomes of a bacterial population using the CRISPR-Cas adaptation system.

While dynamics underlie many biological processes, the ability to robustly and accurately profile time-varying biological signals and regulatory programs remains limited. Here, a framework to store temporal biological information directly into the genomes of a cell population is provided. A “biological tape recorder” was developed in which biological signals trigger intracellular DNA production that is then recorded by the CRISPR-Cas adaptation system. This approach enabled stable recording over multiple days and accurate reconstruction of temporal and lineage information by sequencing CRISPR arrays. A multiplexing strategy to simultaneously record the temporal availability of three metabolites (copper, trehalose, fucose) in the environment of a cell population over time was also developed. This enabled the temporal measurement of dynamic cellular states and environmental changes and suggested new applications for chronicling biological events on a large scale.

A tape recorder converts temporal signals such as analog audio into recordable data written to a tape substrate as it is passed at a set rate across the recorder. Inspired by this temporal data storage scheme (FIG. 1A), a biological realization of the system was developed, referred to as temporal recording in arrays by CRISPR expansion (TRACE). In this framework, a biological input signal is first transformed into a change in the abundance of a trigger DNA pool within living cells. The CRISPR-Cas spacer acquisition machinery is then employed to record the amount of trigger DNA into CRISPR arrays in a unidirectional manner (FIG. 1B). Through this architecture, the presence of an input signal increases the frequency of trigger spacers incorporated into arrays, which constitutes recording of the positive signal. However, in the absence of a signal, reference spacers can still be acquired into arrays at a background rate from sources other than the trigger DNA, such as the genome. These reference spacers serve as pace-denoting markers that are embedded during the recording session, akin to the physical spacing on a tape substrate that represents time intervals.

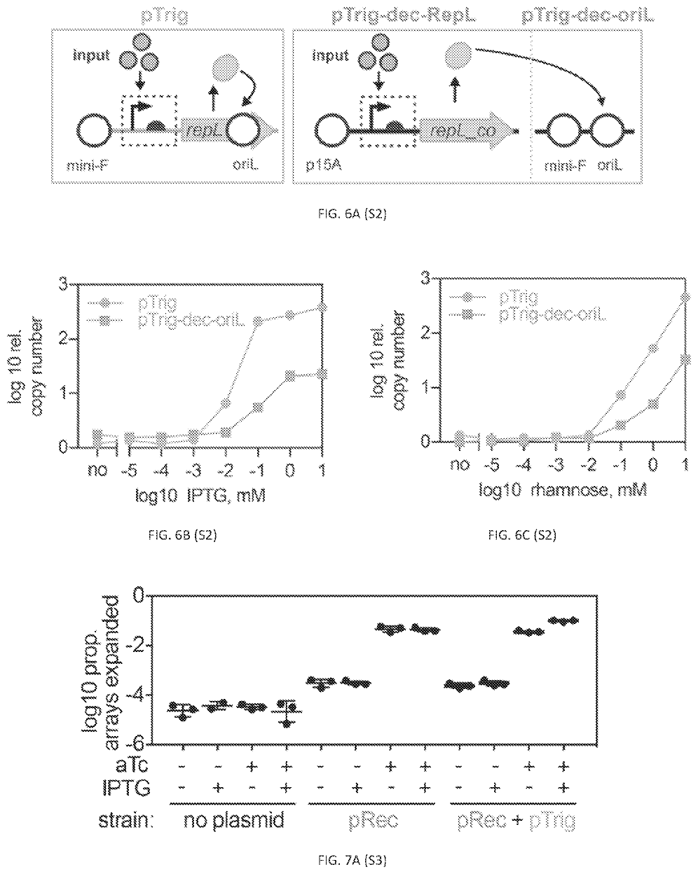

An approach to convert the presence of a biological input into an increase in the abundance of a trigger DNA pool within a population of Escherichia coli cells was explored. A copy number inducible trigger plasmid (pTrig) was utilized, which contained a mini-F origin for stable maintenance and the phage P1 lytic replication protein RepL placed downstream of the Lac promoter. In the presence of the test input signal isopropyl β-D-1-thiogalactopyranoside (IPTG), transcription from the Lac promoter increased and resulting in expression of RepL. The RepL protein subsequently initiated plasmid replication from an origin located within the RepL coding sequence, which in turn increased pTrig copy number (FIG. 1C). Analysis of pTrig by quantitative PCR (qPCR) revealed a 653±5 fold increase in copy number in cells induced with IPTG for 6 hours compared to no induction (FIGS. 1D, 5 and 6). This demonstrated that a biological signal that elicits a transcriptional response can be coupled to alteration of an intracellular DNA pool.

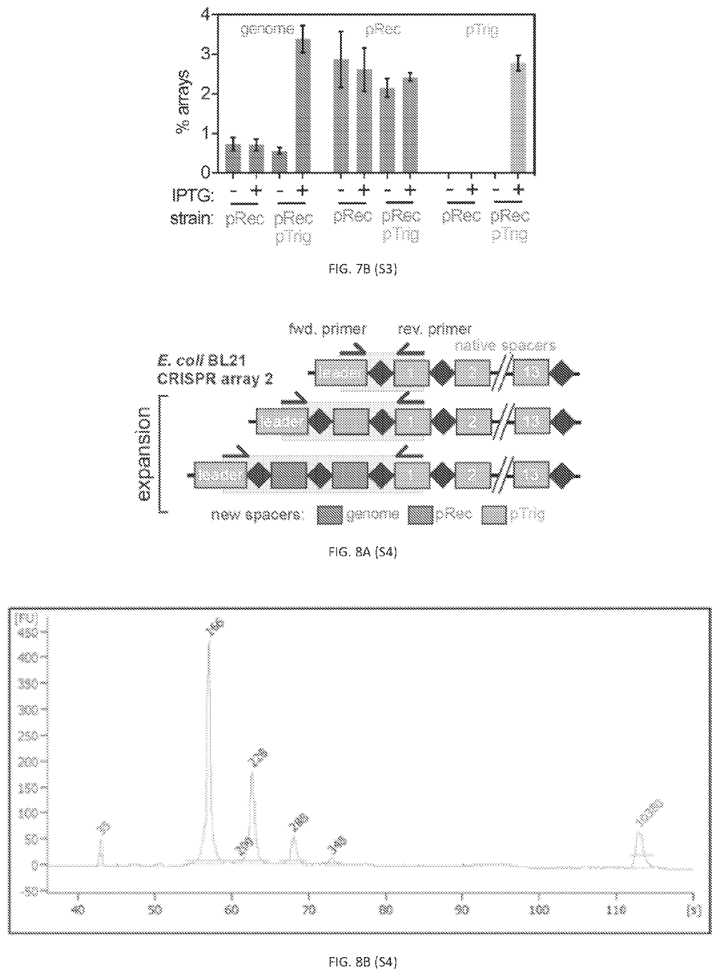

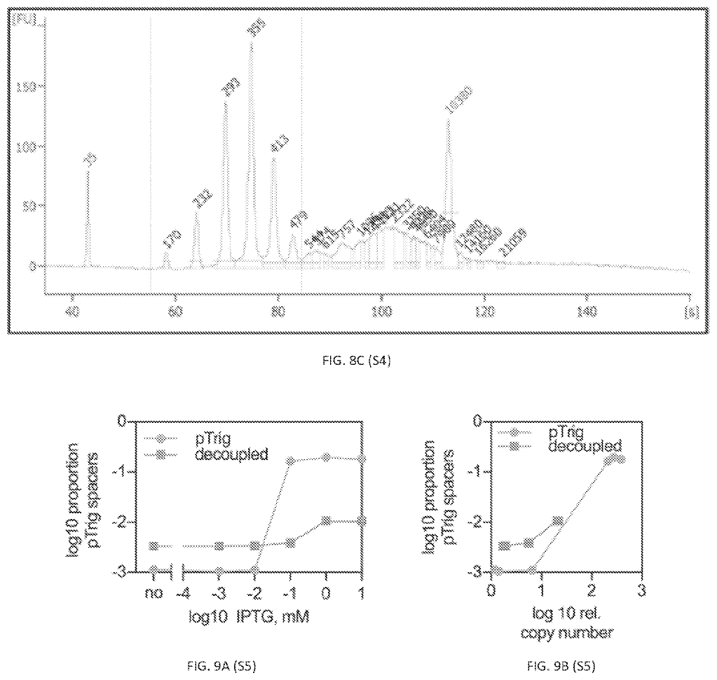

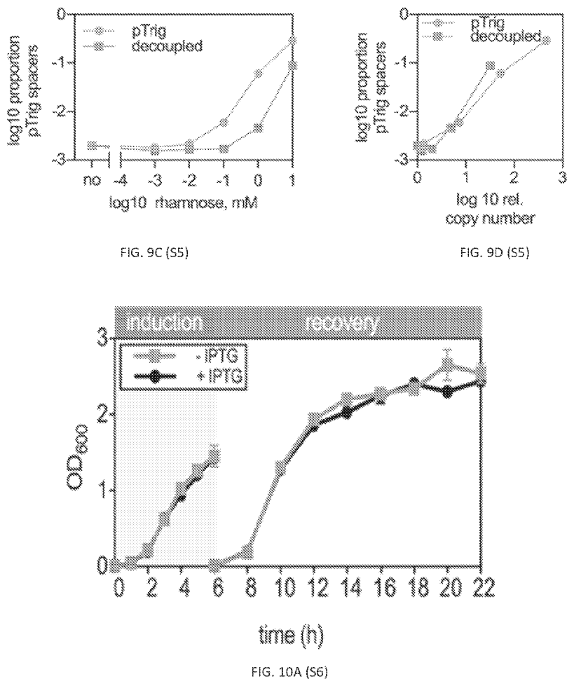

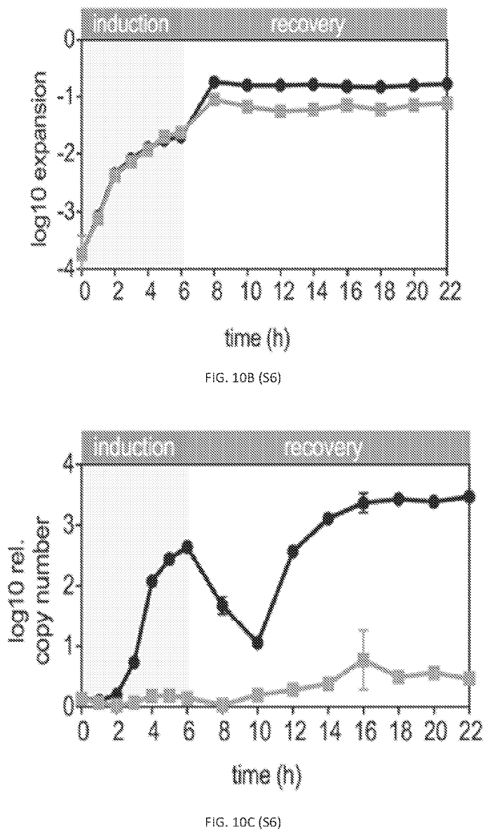

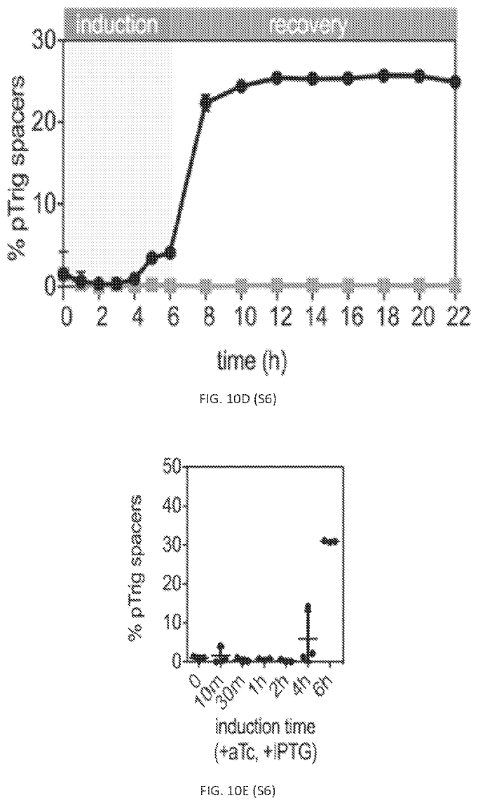

Whether an increase in pTrig copy number could be recorded into CRISPR arrays across a cell population was assessed. Expression of the CRISPR adaptation proteins Cas1 and Cas2 promotes unidirectional integration of ˜33 bp DNA spacers into genomic CRISPR arrays in E. coli. A recording plasmid (pRec) was constructed that expressed Cas1 and Cas2 upon addition of anhydrotetracycline (aTc), which results in spacer acquisition (FIGS. 1E and 7A). Cells with pRec or pRec+pTrig were induced with aTc and with or without IPTG, and their CRISPR arrays were assessed by sequencing to determine the source of newly acquired spacers, either from pRec, pTrig or the genome (FIGS. 1F, 1G and 8). In cells with pRec, spacers were preferentially derived from the pRec plasmid, consistent with enriched spacer acquisitions from plasmids in E. coli documented previously (A. Levy et al., Nature. 520, 505-510 (2015)). Cells with pRec+pTrig, but without IPTG induction, resulted in similar spacer acquisitions and low pTrig spacer incorporation (0.23±0.06% of spacers). However, IPTG induction of pTrig increased overall spacer acquisition (FIG. 7B) and more importantly increased the percentage of pTrig-derived spacers (32.4±0.4% of spacers). This result demonstrated that an induced increase in trigger DNA abundance can be specifically recorded into CRISPR arrays. Different input IPTG concentrations were explored and an increasing relationship between pTrig copy number and the resulting percentage of pTrig derived spacers was observed (FIG. 9). While increased pTrig spacer incorporation could be detected after 4 hours of induction, robust recording was best achieved when the signal persisted for at least 6 hours (FIG. 10).

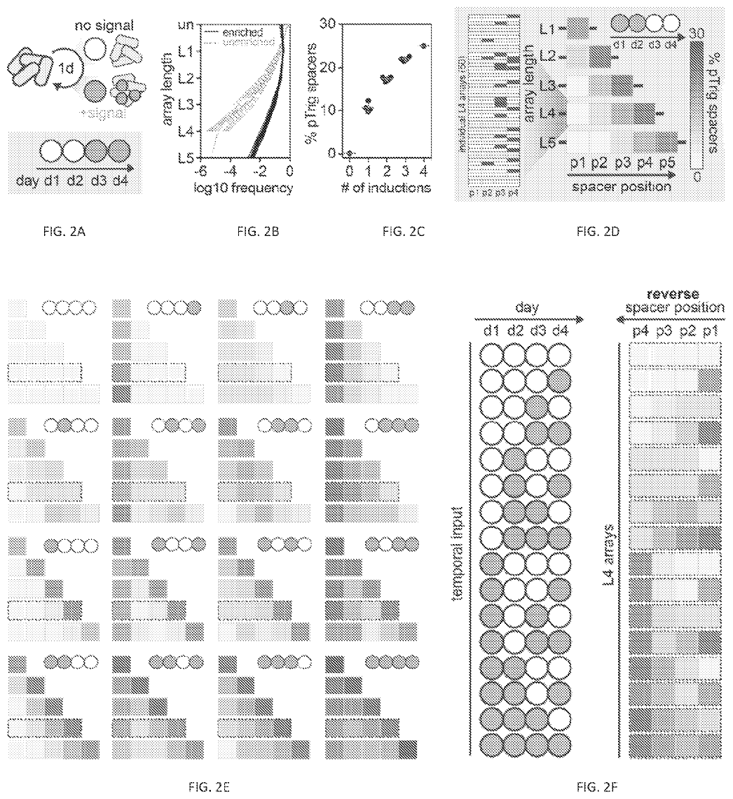

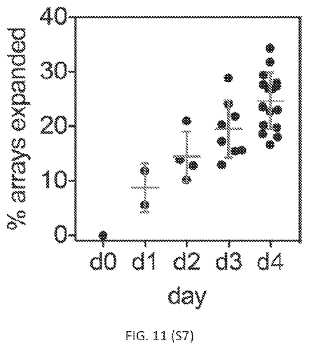

Having assessed the two main components of the system (transformation of a biological signal to increase abundance of an intracellular DNA pool, and capture of the amplified pool into CRISPR arrays), whether TRACE could be used to record biological signals in the temporal domain was tested. A systematic time-course recording experiment was performed in which cells experienced the presence or absence of IPTG across four sequential days (d1-d4) constituting 16 unique temporal signal profiles (FIG. 2A). Sequencing the resulting CRISPR arrays confirmed an overall expansion in array sizes over time (FIG. 11) with 24.7±5.2% of all arrays having incorporated at least one new spacer by d4. On average, ˜1 in 15 arrays acquired a new spacer each day. As expected, arrays with increasing numbers of spacers were detected with decreasing frequency across the population (FIG. 2B). Since longer arrays contained more temporal information, a size enrichment protocol was also implemented that facilitated the analysis of arrays with up to 5 new spacers (FIG. 2B).

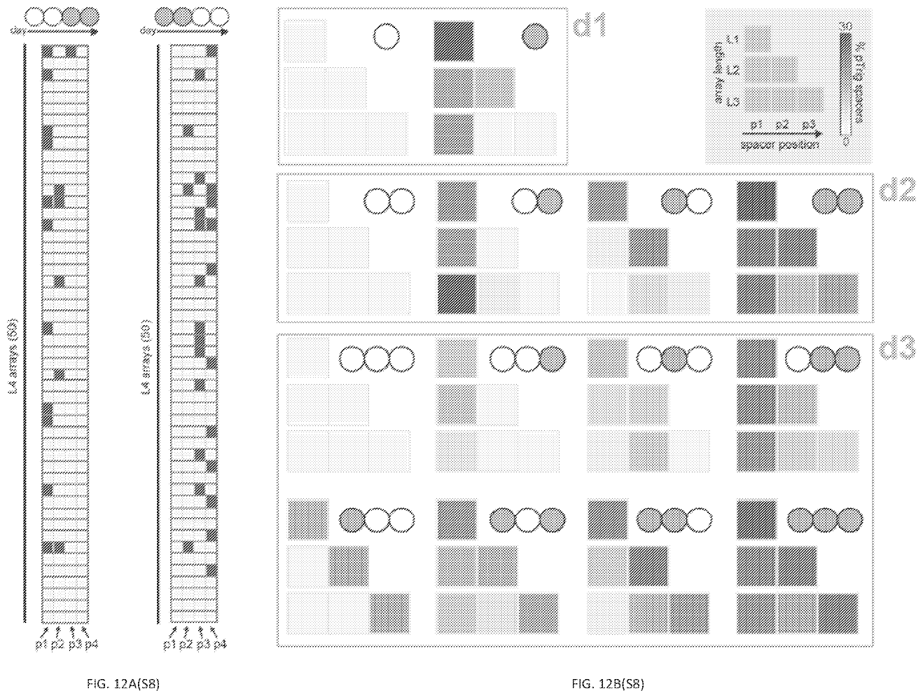

For TRACE to function as a useful biological tape recorder, the spacer identity (reference or trigger) and ordering within CRISPR arrays should correlate with the actual temporal signal profile. The system was able to act as a simple signal counter—the total percentage of pTrig spacers increased proportionally with the number of times the signal was present in the signal profile (FIG. 2C). Next, pTrig spacer incorporation and ordering in CRISPR arrays was analyzed. For example, individual arrays from a sample receiving the IPTG profile [on, on, off, off] were variable but displayed an overall enrichment of pTrig spacers at distal positions in the array (FIGS. 2D and 12A). To visualize these incorporation patterns across each of the 16 signal profiles, for arrays of different lengths (L1 to L5), the population average of pTrig spacers at each spacer position was calculated (p1 to p5, FIGS. 2D, 2E, and 12B). Strikingly, these patterns of pTrig frequencies exhibited a high degree of correspondence to their respective temporal signal profiles when considered in reverse (e.g. oldest to newest acquired spacers, FIG. 2F), which suggested the successful recording of temporal biological signals.

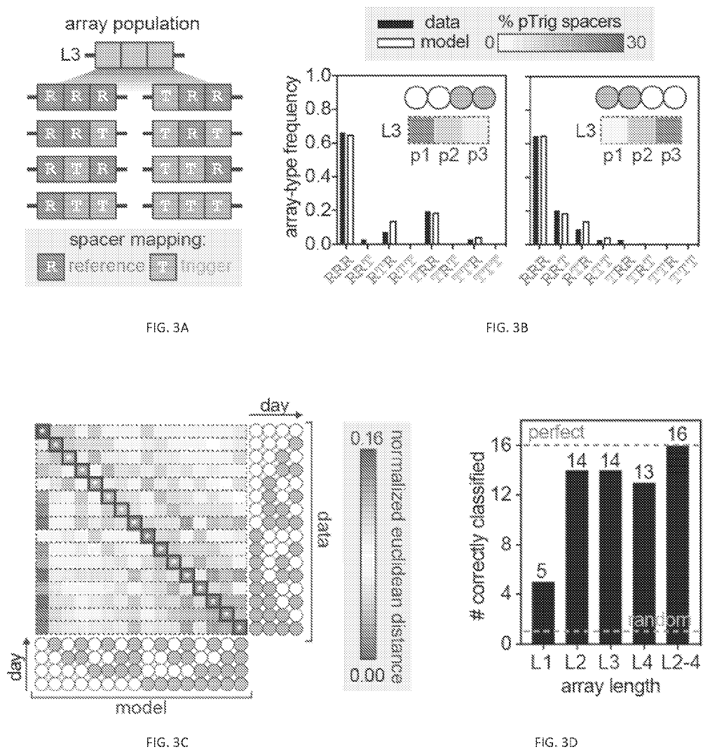

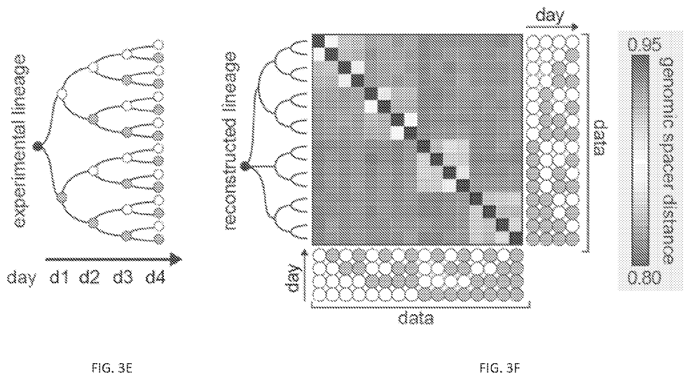

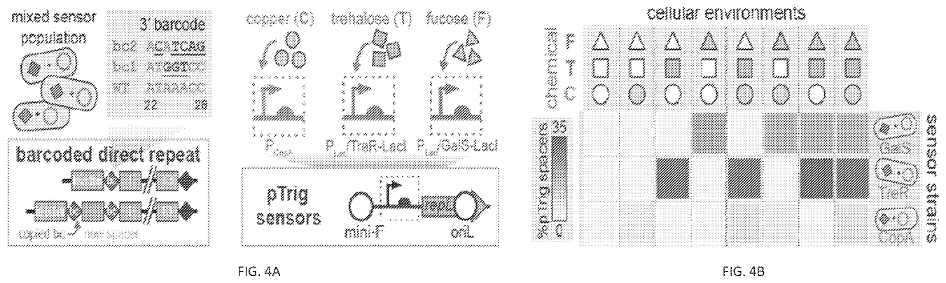

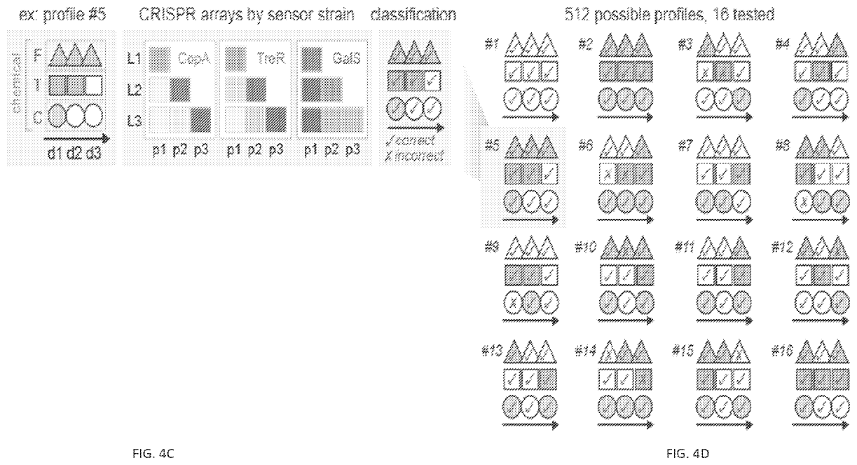

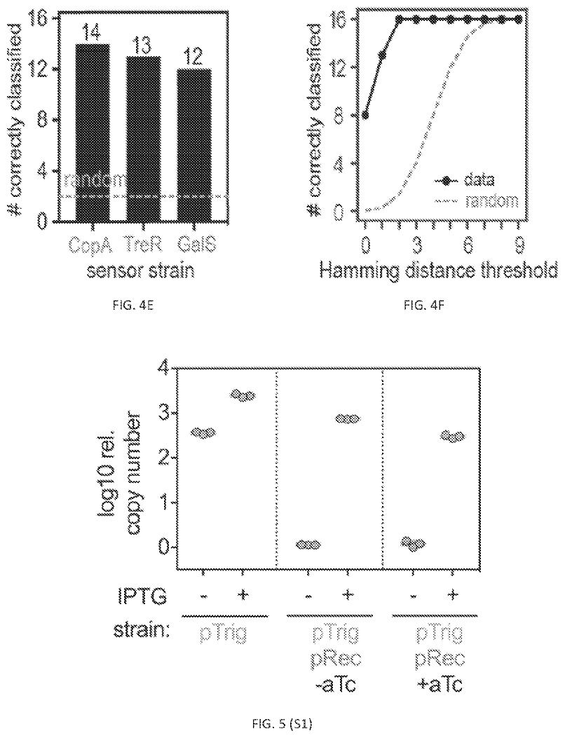

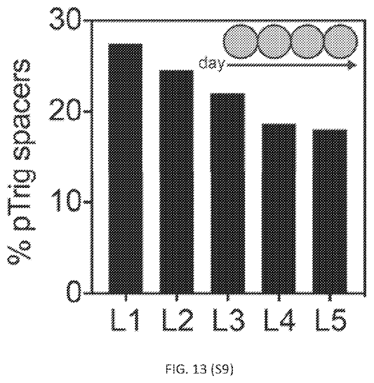

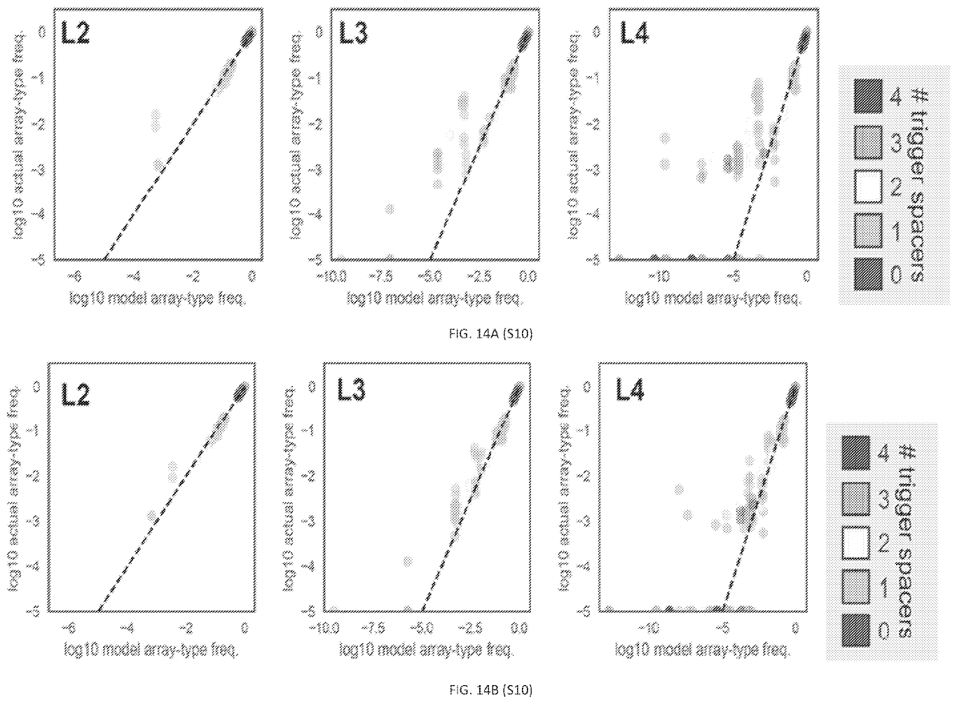

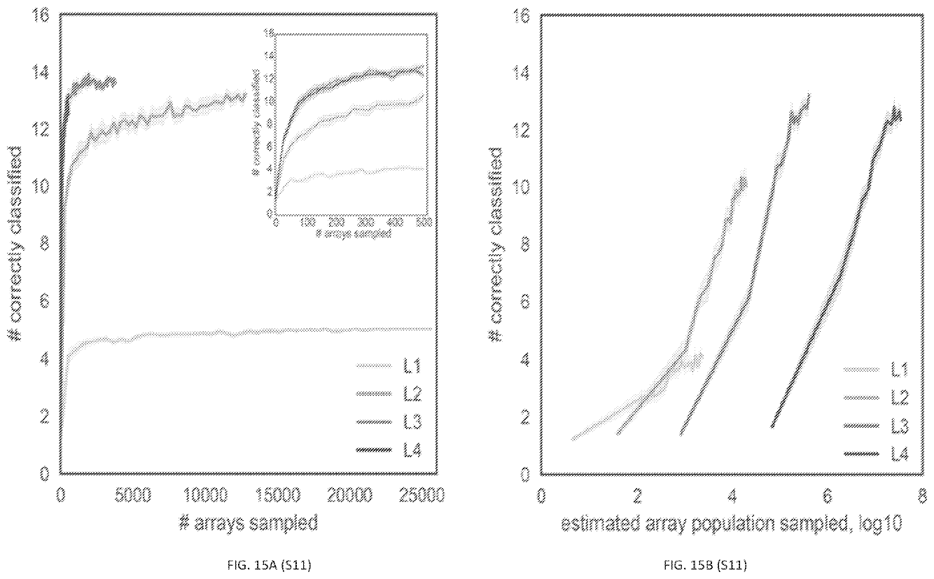

To improve the interpretation of TRACE data, a method for accurate and automated inference of the input temporal signal profiles from recorded CRISPR arrays was explored. It was hypothesized that the array expansion process could be modeled to yield a useful classification scheme for matching an observed pattern of arrays to its corresponding signal profile. To test this approach, a cell population's repertoire of CRISPR arrays was defined as a distribution of “array-types”. Array-types constitute all possible array configurations across all array lengths with either reference (R) or trigger (T) spacers occupying each spacer position (FIG. 3A). A simple analytical model of the CRISPR expansion process was then developed for calculating the expected frequencies for all array-types given a signal profile. Four constants are needed to parameterize the model for each array length: the rates of array expansion and pTrig incorporation per recording interval, given the presence or absence of a signal (FIG. 13, Table 5). Using this model, the expected distributions of array-types for all 16 temporal signal profiles were calculated and compared with those from experimentally recorded arrays. Interestingly, the predicted and observed array-type distributions matched closely (FIG. 14). For example, for two signal profiles of equal number of inductions but differing temporal ordering, the models yielded distinctive array-type distributions that appeared to recapitulate their corresponding experimental data (FIG. 3B).

To quantitatively compare and classify the observed data with model array-type distributions, all pairwise Euclidean distances between them were calculated. An observed CRISPR array population was assigned to the most probable signal profile based on the data-model pair with the shortest Euclidean distance (FIG. 3C). Using L1 arrays only, which do not contain any temporal information, only 5 of 16 signal profiles could be correctly classified. In contrast, using L2 to L4 array-types individually resulted in much higher accuracy of assignments (13-14 of 16 correct). When L2-L4 array-types were simultaneously used together, all 16 populations were perfectly classified with their correct temporal signal profiles (Methods, FIG. 3D). A few hundred arrays of a given length, corresponding to minimum populations of ˜10⁵ total arrays were required to recapitulate reasonable classification accuracy (FIG. 15). Temporal signals could be recorded and subsequently reconstructed with high accuracy from CRISPR arrays using this model of the expansion process.

Beyond simply assigning spacer identity as reference or trigger, it was hypothesized that spacer sequences themselves may additionally contain population lineage information given the large pool of potential spacers. In the time-course recording experiment, cell populations were experimentally split into sub-populations each day, which resulted in a defined branching history of the 16 populations (FIG. 3E). Interestingly, by performing lineage reconstruction using a simple metric to assess spacer repertoire distance between populations (See Methods), the entire experimental population lineage was reconstructed with near perfect accuracy (FIG. 3F).

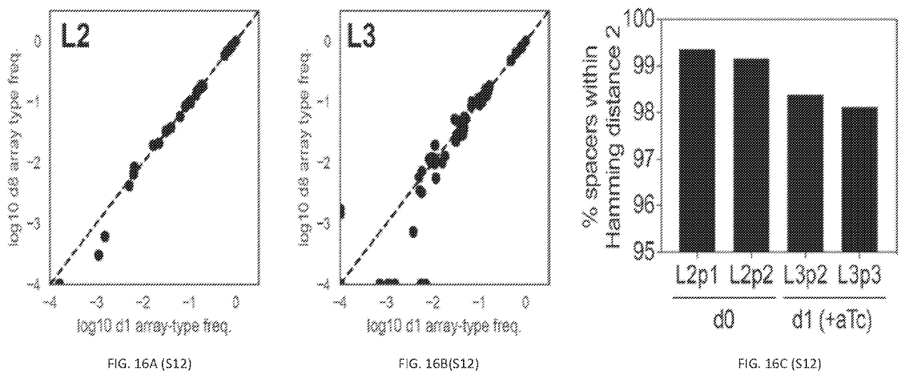

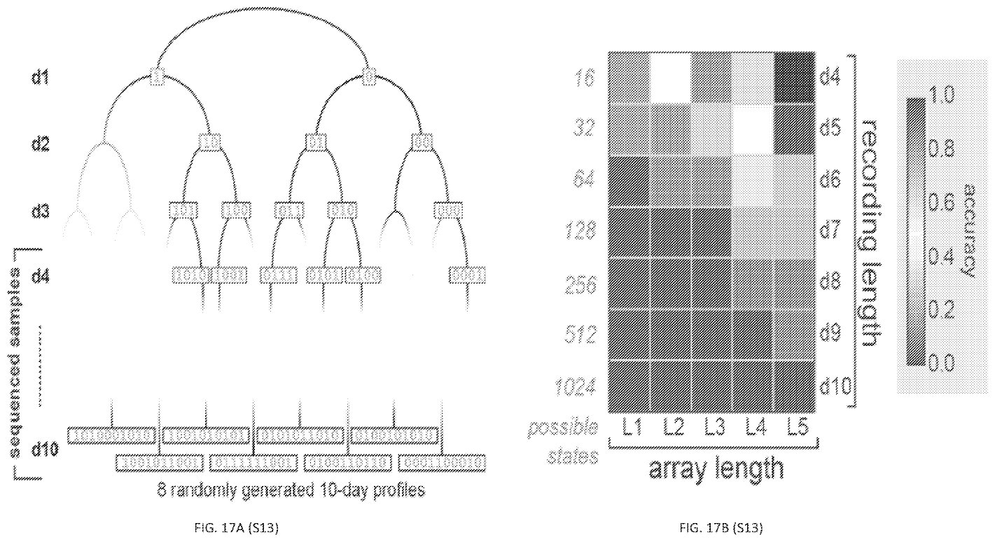

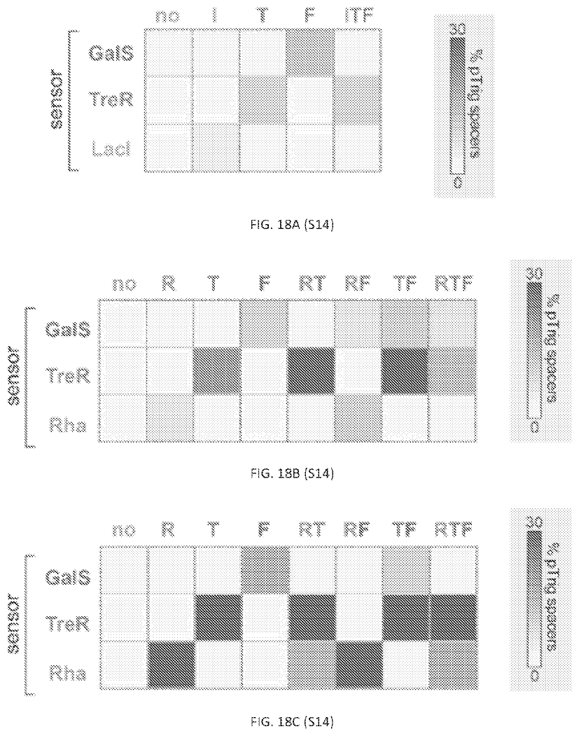

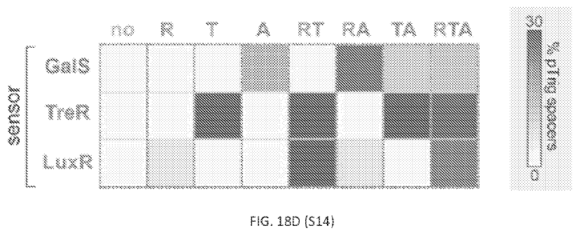

To further characterize the recording performance of TRACE, the stability of stored information and the potential for longer-term recordings was assessed. Propagation of recordings stored within cell populations over 8 days (˜50 generations) did not appear to alter array-type distributions (FIGS. 16A and 16B), while induction of recording showed negligible loss of previously acquired spacers (FIG. 16C). Thus, these results demonstrated stable data storage.

Recording experiments on selected temporal signal profiles were repeated for 10 days, which showed reasonable reconstruction accuracy up to 6 days (4 of 7 correctly classified, FIG. 17). In general, longer arrays increased the accuracy of signal profile reconstruction during longer recording sessions, which suggested that longer-read sequencing may further increase the performance of long-term recording analysis.

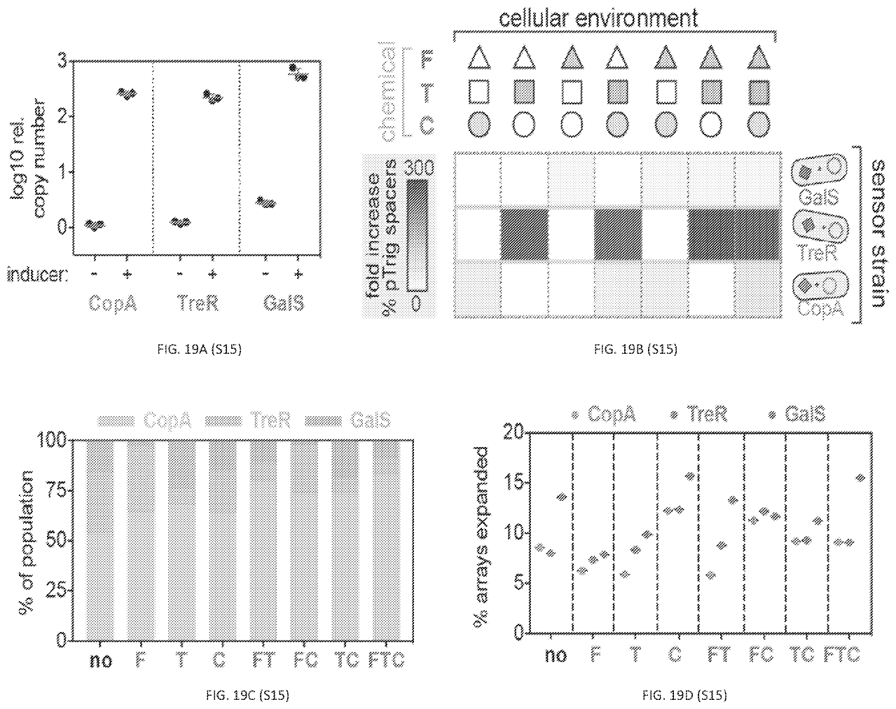

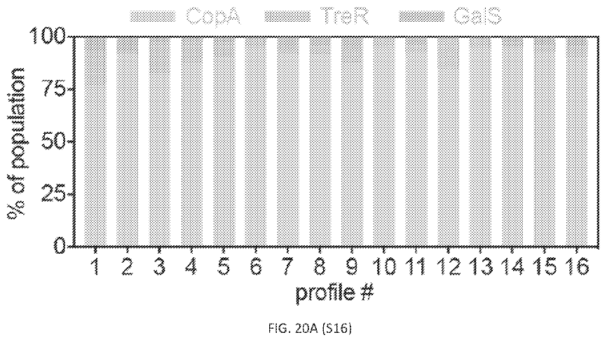

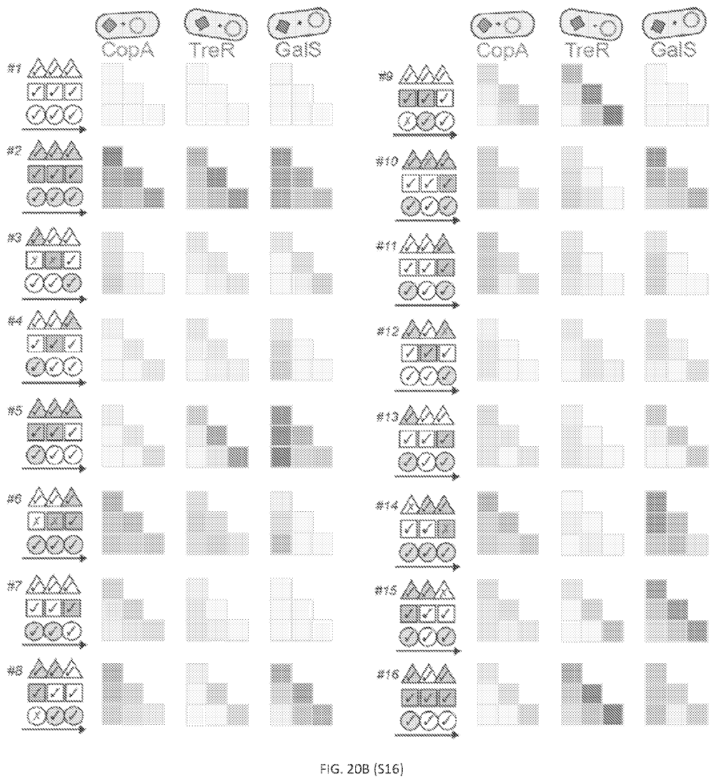

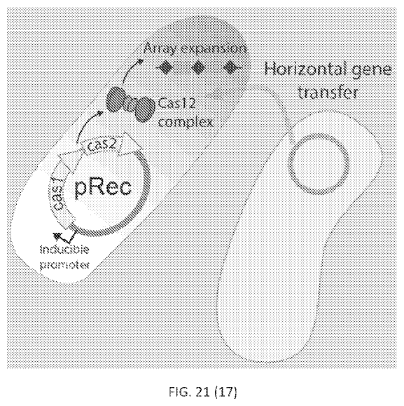

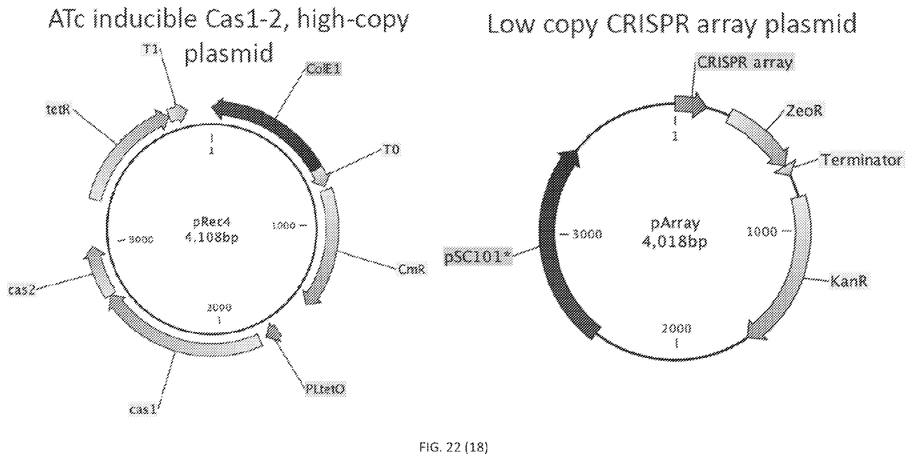

A multiplexing strategy was devised wherein various pTrig sensor systems could be associated with uniquely barcoded CRISPR arrays within a cell population (FIG. 4A). Specifically, the 3′ direct repeat (DR) sequence was mutated, which, based on previous studies, would not affect spacer integration as a barcode. This allowed for multiplexing with no modification to the sequencing protocol. More importantly, this enabled more stringent calling of barcodes since the DR sequence is duplicated during each spacer incorporation event. Using MAGE (H. H. Wang et al., Nature. 460, 894-898 (2009)), strains with new genomic DR barcodes were generated. In distinct barcoded strains, different sensors were coupled to pTrig and their performance was screened (FIG. 18). Three orthogonal and robust biosensors that detected biologically meaningful chemicals, copper (heavy-metal contaminant), trehalose (dietary sugar metabolite) and fucose (associated with mammalian gut infection), were eventually selected for multiplex recording experiments. To assess the capacity for multi-channel recording, cell populations containing a mix of all three strains were exposed to all 8 combinations of the 3 input chemicals. The resulting CRISPR arrays were sequenced and demultiplexed using the DR barcodes. Each sensor strain displayed robust increase of pTrig-derived spacers (>24 fold) only in the presence of their cognate input (FIGS. 18B and 19). Importantly, these results indicated modular compatibility of TRACE for multi-channel recording with a variety of sensing systems, including engineered sensors or native promoters with endogenous transcription factor expression.

To explore multiplex temporal recording, the three-strain sensing system was used to perform a time-course exposure experiment over three days. Cell populations were exposed to 16 selected temporal signal profiles of 512 possible profiles, and resulting CRISPR arrays were sequenced. Sensor strains fluctuated in their final abundance but were maintained at sufficient levels to enable CRISPR array analysis (FIG. 20). Models for each sensor were parameterized individually as before and the exposure history of each of the three inputs was inferred individually for all 16 populations by classification against model predictions (FIG. 4C). 14, 13 and 12 of the 16 signal profiles for the copper, trehalose, and fucose sensors, respectively, were correctly classified (FIGS. 4D and 4E). Classification accuracy of all three inputs simultaneously was assessed by the Hamming distance threshold to the actual temporal signal profiles; 8 of 16 profiles were perfectly classified and the rest were within Hamming distance 2 (FIG. 4F), implying that even incorrect predictions were close to actual signal profiles. Together, these results demonstrated accurate multichannel recording with the TRACE system.

TRACE can be utilized to record metabolite fluctuations, gene expression changes, and lineage-associated information across cell populations in difficult-to-study habitats such as the mammalian gut or in open settings such as soil or marine environments. The system could employ inducible intracellular DNA production systems in parallel (See, for example, J. Elbaz, P. Yin, C. A. Voigt, Nature Communications. 7, 11179 (2016), incorporated herein by reference) and other CRISPR-Cas adaptation machinery (S. A. Jackson et al., Science. 356, eaa15056 (2017) and S. Silas et al., Science. 351, aad4234 (2016), incorporated herein by reference in their entirety), which may be needed for extension to other bacteria (or even eukaryotes) and to increase the temporal resolution of recording beyond the levels demonstrated here (6 hours, ˜45 μHz). The system could be further optimized by increasing the spacer incorporation rate (R Heler et al., Molecular Cell. 65, 168-175 (2017), incorporated herein by reference in its entirety), increasing the sequencing length (e.g. by nanopore sequencing), and improving reconstruction algorithms. These advances could further facilitate biological recording of inputs across many signal channels, with higher temporal resolution, and in smaller populations possibly down to single cells. TRACE and future strategies for massively parallel recording of biological states should greatly advance the ability to delineate and understand complex cellular processes across time.

### Materials and Methods

### Plasmid Construction

All plasmids (Table 1) were constructed via the Golden Gate method (See, C. Engler, R. Kandzia, S. Marillonnet, PLoS ONE. 3, e3647 (2008), incorporated herein by reference in its entirety) with the NEB 10-beta cloning strain (NEB, C3019H) and were verified via Sanger sequencing (Eton Bioscience, Genewiz). All plasmids are deposited at Addgene. The RBS calculator Nature Biotechnology. 27, 946-950 (2009), incorporated herein by reference in its entirety) and Anderson library of promoters (Available at the Registry of Standard Biological Parts (parts.igem.org/Promoters/Catalog/Anderson)) were utilized as annotated on plasmid maps.

TABLE 1
Plasmids used in this study.

| | | resistance | | |
|---|---|---|---|---|
| ref name | plasmid | marker | origin | description |
| pRS001 | pRec | CmR | ColE1 | PTet-cas12, tetR, lacI |
| pRS002 | pRec-GalS | CmR | ColE1 | PTet-cas12, tetR lacI-galS |
| pRS003 | pRec-TreR | CmR | ColE1 | PTet-cas12, tetR, lacI-treR |
| pRS004 | pRec ΔLacI | CmR | ColE1 | PTet-cas12, tetR |
| pRS005 | pTrig | KanR | mini-F | PLac-repL |
| pRS006 | pTrig-CopA | KanR | mini-F | PCopA-RiboJ-B0034-repL |

| ref name | plasmid | map |
|---|---|---|
| pRS001 | pRec | benchling.com/s/seq-A9McFCX7BXXXI9vSBrRe |
| pRS002 | pRec-GalS | benchling.com/s/seq-I8zistPSzTIXMneP5V4h |
| pRS003 | pRec-TreR | benchling.com/s/seq-30jc7WJzBGX8fNKZp7Pz |
| pRS004 | pRec ΔLacI | benchling.com/s/seq-cv8by55ejdFb4xCZD4d1 |
| pRS005 | pTrig | benchling.com/s/seq-ISWVXtHWPPuY5zCBNceM |
| pRS006 | pTrig-CopA | benchling.com/s/seq-JBz03HXz2h1sDNJ4ob9P |

The pTrig plasmid was generated from pSB2K3-BBa_J04450 (iGEM 2016 distribution), which itself was derived from the pSCANS vector. To construct pTrig, the BBa_J04450 (RFP) sequence and Biobrick multiple cloning site were removed; the resulting plasmid contains the mini-F origin and replication machinery, P1 lytic replication element RepL placed downstream of an IPTG-inducible Lac promoter, and kanamycin resistance marker.

The pRec plasmid was generated by placing the E. coli cas1-cas2 cassette (amplified from NEB 10-beta) downstream of the P_{LTeto-1} promoter (See R. Lutz, H. Bujard, Nucleic Acids Research. 25, 1203-1210 (1997), incorporated herein by reference in its entirety) on a ColE1 plasmid containing chloramphenicol resistance marker and constitutively expressed TetR and LacI (LacI is required to repress the Lac promoter on pTrig, see FIG. 5).

For the CopA sensor, a derivative of the pTrig plasmid (pTrig-CopA) containing the E. coli BL21 CopA promoter (100 bp upstream sequence) with RiboJ (C. Lou, B. Stanton, Y.-J. Chen, B. Munsky, C. A. Voigt, Nature Biotechnology. 30, 1137-1142 (2012), incorporated herein by reference in its entirety) and B0034 RBS was constructed. This was utilized with a derivative of the pRec plasmid without LacI (pRec ΔLacI).

For the GalS and TreR sensors, derivatives of the pRec plasmid containing LacI chimeric transcription factors (pRec-TreR, pRec-GalS) were constructed by swapping the LacI ligand binding domain with either the TreR or GalS ligand binding domains and then subsequently introducing point mutations that have been characterized to improve sensor performance (TreR: V52A; GalS. Q54A, E232K). These pRec variants were then utilized with the pTrig plasmid.

Chromosomal Alteration of Strains with MAGE

Given utilization of Lac chimeric transcription factors (GalS, TreR), a variant of the E. coli BL21 strain lacking endogenous expression of LacI was generated to prevent interaction with the sensing systems. The MODEST tool was utilized to design a recombineering primer (MAGE_tKO_lacI, Table 3) to perform a translational knockout of chromosomal LacI by introduction of three stop codons into the beginning of the lacI coding sequence. Briefly, the BL21 strain was transformed with pKD46 (K. A. Datsenko, B. L. Wanner, Proc. Nalt. Acad. Sci. U.S.A. 97, 6640-6645 (2000), incorporated herein by reference in its entirety) and grown at 30° C. with 50 μg/mL Carbenicillin (Fisher BP2648). An overnight culture of this strain was back-diluted and grown for 30 min, 0.5% arabinose was added, and the culture was grown to approximately OD600=0.6. 1 mL of cells were then placed on ice and washed with nuclease-free water 3 times, resuspended in 2.5 μM oligonucleotide at a volume of 50 μL, and subjected to electroporation. Cells were then recovered for 1 hour at 30° C. This process constituted one round of recombineering; after this procedure cells were plated on LB-agar with antibiotics and X-gal (200 μg/mL, Thermo FERR0404) and grown at 30° C. Resulting clones were screened for loss of LacI expression by beta-galactosidase assay (loss of LacI expression de-represses LacZ), and a resulting clone was verified to contain the correct chromosomal alteration by Sanger sequencing. This strain was hereafter denoted BL21 LacI_tKO.

Oligo recombineering was also utilized to introduce barcodes into the genomic CRISPR array first direct repeat (DR) sequence. A recombineering primer (MAGE_BL21_DR, Table 3) was designed to mutagenize the distal 7 bp of the DR sequence (inadvertently, the first base pair of the first native genomic spacer was also targeted for mutagenesis, resulting in 8 bp total targeted for mutagenesis). The BL21 LacI_tKO strain, still harboring pKD46 was subjected to five rounds of oligo recombineering as described above. The resulting cell population was then subjected to heat shock at 42° C. for 1 hour to promote loss of pKD46 and recovered overnight at 37° C. in LB without antibiotics; a cryostock of the population (15% glycerol) was saved for subsequent screening for clones with barcoded DR sequences.

Experimental Conditions (Induction of pRec and pTrig)

All testing was conducted in E. coli BL21 (NEB C2530H), a strain that contains two genomic CRISPR arrays but lacks cas interference machinery. For induction experiments with the Lac sensor, the E. coli BL21 strain was transformed with appropriate plasmids (pRec, or pRec+pTrig) via electroporation (Table 2). A single colony was picked and grown to stationary phase and a cryostock (15% glycerol) was created for storage at −80° C.

The general experimental workflow of an induction experiment was as follows:
1. A culture tube (Thomas Scientific 110158PL-TS) containing 3 mL autoclaved LB-Lennox (BD 240230) and appropriate antibiotics at indicated final concentrations (pRec: chloramphenicol 34 μg/mL [EMD Millipore Omnipur 3130, diluted in 100% ethanol], pTrig: kanamycin 50 μg/mL [Fisher BP906-5, diluted in nuclease free water]) was inoculated from the culture glycerol stock and grown overnight (>12 hours) at 37° C. in an Innova44 incubator shaker at 230 rpm.
2. The next day, this culture was diluted 1:100 into a new tube containing 3 mL LB media and appropriate antibiotics and allowed to grow in the same culture conditions for 2 hours to bring cultures into exponential phase.
3. This culture was then diluted 1:100 into a new tube containing 3 mL LB media, appropriate antibiotics, and appropriate anhydrotetracycline (aTc) and isopropyl β-D-1-thiogalactopyranoside (IPTG) inducers at indicated final concentrations (aTc: 100 ng/mL [Cayman 10009542, diluted in 100% ethanol], IPTG: 1 mM [Thermo R0392, diluted in nuclease free water]). This culture was then allowed to grow in the same culture conditions for 6 hours.
4. Finally, culture from this tube was diluted 1:100 into a new tube containing 3 mL LB media and appropriate antibiotics, and allowed to recover in the same culture conditions overnight for 16 hours.
5. At the conclusion of the experiment 500 μL of culture was transferred to a 1.5 mL tube (VWR 20170-333), the tube was spun down (15,0000 rpm, 30s) to pellet cells, media was removed, and the pellet was stored at −20° C. for subsequent analysis.

### Experimental Conditions (Temporal Recordings)

For 4 day temporal recording experiments the induction procedure as above was utilized, but after the first day, recovery cultures from the previous day were diluted, starting at step 2 of protocol. All cultures were exposed to aTc and received no IPTG or 1 mM IPTG. Samples were collected from each recovery culture for analysis. As noted, the experiment was performed in a branched manner, in that a single culture from a previous day was used to inoculate two daughter cultures (one receiving IPTG inducer, one not).

For the 10 day temporal recording experiment, 8 exposure profiles were randomly generated and conducted in a similar manner over the course of 10 days (1010001010, 1001011001, 1001010101, 0111111001, 0101011010, 0100110110, 0100101010, 0001100010; 1 indicates induction and 0 indicates no induction) and samples were collected from d4 to d10.

The experiment was also performed in a branching manner as above; therefore given that the starting substring of some samples were shared, some shorter time points had less than 8 samples (d4-d5:6, d6:7, d7-d10:8).

### Experimental Conditions (Multiplexed Recording)

To generate barcoded strains with the three additional sensors for the multiplexed recording experiment, 100 μL of the BL21 LacI_tKO with mutagenized DR cryostock was re-inoculated into an overnight culture of LB with no antibiotics. The appropriate pRec and pTrig plasmids for the TreR and GalS sensors (Table 2) were transformed into this population via electroporation. Colonies were then picked and screened for mutated DR sequence via Sanger sequencing. This yielded mutated DR sequences for TreR (ATGGTCC (SEQ ID NO: 33), underline denotes altered sequence from WT) and GalS (ACATCAG (SEQ ID NO: 34)). The GalS strain also contained a mutation in the first basepair of the first native genomic spacer (G to A) due to inadvertent targeting; however, this did not affect analysis given thresholds utilized in matching during sequencing analysis. The TreR background strain is referred to as BL21 LacI_tKO DR_mut_1 and the GalS background strain BL21 LacI_tKO DR_mut_2. The plasmids for the CopA sensor (Table 2) were separately transformed into E. coli BL21. The three sensor strains were then grown separately in filter sterilized M9 media with appropriate antibiotics (1× M9 salts [BD 248510], 0.8% (wt/vol) glycerol [Fisher G33-1], 0.2% (wt/vol) casamino acids [BD 223120], 2 mM MgSO4 [Sigma-Aldrich 230391], 0.1 mM CaCl₂ [Sigma-Aldrich C1016]) and a cryostock (15% glycerol) was created for storage at −80° C.

TABLE 2
Strains used in this study.

| strain | plasmid1 | plasmid2 |
|---|---|---|
| BL21 | | |
| BL21 | pRec | |
| BL21 | pRec | pTrig |
| BL21 | pRec ΔLacI | pTrig-CopA |
| BL21 LacI_tKO DR_mut_1 | pRec-TreR | pTrig |
| BL21 LacI_tKO DR_mut_2 | pRec-GalS | pTrig |

The general experimental workflow followed the temporal recording induction protocol with minor modification. All multiplexed recordings were conducted in M9 media. The three strains were grown overnight separately, optical density was measured, and the three strains were pooled at equal densities. The initial dilution (step 2) was 1:10 rather than 1:100 given slower growth rate in M9 media compared to LB. Before recovery (step 4), cells were spun down (15,0000 rpm, 30s), media was removed and cells were resuspended in 1 mL of fresh media to remove any residual inducer. Inducers for the three sensors were as follows, CopA: 100 μM copper sulfate (Sigma-Aldrich 209198), TreR: 1 mM trehalose (Sigma-Aldrich T9531), GalS: 1 mM fucose (Sigma-Aldrich F8150).

qPCR Assay for pTrig Copy Number

A qPCR plasmid copy number assay was utilized to assay pTrig copy number. Briefly, 18 μL of a qPCR master mix (10 μL 2× KAPA SYBR Fast qPCR Master Mix [KAPA KK4601], 0.6 μL 10 μM forward primer, 0.6 μL 10 μM reverse primer, 6.8 μL nuclease free water) was dispensed into a 96 well qPCR plate (Bio-Rad HSL9905) and 2 μL of template as prepared during sequencing library preparation (see protocol below) was added. Two qPCRs were performed, the first with primers targeting pTrig and the second with primers targeting the genome (see Table 3 for sequences). Both primer pairs were confirmed to have >90% amplification efficiency. The PCR plates were sealed with optically transparent film (Bio-Rad MSB1001) and were placed on a qPCR system (Bio-Rad CFX96) and subjected to following cycling conditions: 95° C. 3 min, 39 cycles: 95° C. 3 s. 60° C. 20 s, 72° C. 1 s and acquisition. The Cq values were determined via the manufacturer's software, and pTrig relative enrichment was calculated with the delta delta Cq method (e.g. 2\hat{ }(−1*(pTrig_Cq−16S_Cq)), normalized to the lowest value). A melt curve was performed to ensure that only a single amplification product was present.

TABLE 3
Primers used in this study,

| Primer | sequence (5′-3′) |
|---|---|
| MAGE_tKO_ | G*G*A*A*GAGAGTCAATTCAGGGTGGTGAATGTGAAAC |
| LacI | CAGTATAGTGATAAGATGTCGCAGAGTATGCCGGTGTCT |
| | CTTATCAGACCGTTTC (SEQ ID NO: 3) |
| MAGE BL21_ | G*G*G*GAACACCCGTAAGTGGTTTGAGCGATGATATTT |
| DR | GTGCTNNNNNNNNCCCCGCTGGCGCGGGGAACACTCTAA |
| | ACATAACCTATTATT (SEQ ID NO: 4) |
| genome_fwd | GCGAGCGATCCAGAAGATCT (SEQ ID NO: 5) |
| genome_rev | GGGTAAAGGATGCCACAGACA (SEQ ID NO: 6) |
| pTrig fwd | CGCTCTATGATCCAGTCGATTT (SEQ ID NO: 7) |
| pTrig rev | TCCGTATGCCATGCGTTTAT (SEQ ID NO: 8) |

For the MAGE_tKO_LacI primer, underlined bases indicate mismatch with genomic LacI sequence. For the MAGE_BL21_DR primer, underlined bases indicate mismatch with genomic sequence designed to barcode individual arrays (note the last N base erroneously targets the first base pair of the first genomic spacer in the array). * indicates that the base immediately preceding symbol is phosphorothioated.

### Design of Custom CRISPR Array Sequencing Scheme

The custom sequencing scheme enabled highly efficient use of illumina read lengths (up to 5 expanded spacers with a 300 cycle sequencing kit) by avoiding re-sequencing of primer sequences as required with most two-step amplification schemes. To design these primers for CRISPR BL21 sequencing (referred to as “CB”), a forward primer targeting the BL21 array I leader sequence and a reverse primer targeting the array I first native genomic spacer were utilized. The forward primer was linked to an Illumina P5 sequence and barcode sequence; a series of 8 were generated (e.g. CB501-CB508). The reverse primer was linked to an Illumina P7 sequence and barcode sequence; a series of 12 were generated (e.g. CB701-CB712). All barcode sequences were derived from Illumina Nextera indices. The combination of 8×12 primers allowed for 96 samples to be uniquely barcoded via dual indexing in a single sequencing run. Custom read 1 (CBR1) and index 1 (CBI1) sequencing primers were also generated. All primer sequences can be found in Table 4. All primers in this study were obtained from IDT with normal desalting purification.

### CRISPR Array Sequencing Library Preparation Protocol

To perform sequencing of CRISPR arrays from populations of cells, a library preparation and sequencing pipeline consisting of three steps: (1) gDNA preparation, (2) PCR amplification and (3) sample pooling, purification, and quality control was developed.

To purify gDNA from cell pellets obtained at the end of an experiment, a modified protocol utilizing the prepGEM Bacteria kit (ZyGEM PBA0500; VWR 95044-082) was developed. Cell pellets were removed from storage at −20° C. in 1.5 mL tubes and resuspended in 100 μL of TE (10 mM Tris-HCl pH 8.0 [Fisher BPI758], 1 mM EDTA pH 8.0 [Sigma-Aldrich 03690] in nuclease free water [Ambion AM9937]). 10 μL of the resulting suspension was pipetted into a 96-well skirted PCR plate (Eppendorf 951020401). 20 μL of a prepGEM master mix (0.30 μL prepGEM enzyme; 0.30 μL lysozyme enzyme, 3.0 μL 10× Green Buffer, 16.4 μL nuclease free water) was then added to each well with a multichannel pipette, and the plate was heat sealed (Vitl V901004 and Vitd V902001). The plate was then spun down for 30 seconds on a plate microfuge (Axygen C1000-AXY) and incubated on a PCR thermocycler (Bio-Rad S1000) with the following program: 37° C. 15 min, 75° C. 15 min, 95° C. 15 min, 4° C. infinite. 70 μL of TW (10 mM Tris-HCl pH 8.0 in nuclease free water) was then added to each well with a multichannel pipette.

To prepare uniquely barcoded amplicons for each sample, PCR amplification was performed using the CB50X and CB7XX sequencing primers (Table 4). First, a master primer-plate was prepared by arraying the CB50X primers across rows of a 96-well PCR plate and CB7XX primers down columns of the same 96-well PCR plate at a final concentration of 10 μM for each primer in 50 μL. Thus, each well contained a unique combination of CB50X and CB7XX primers. A PCR reaction was then set up for each sample by pipetting 2 μL of the mix from the master primer-plate, 5 μL of gDNA from prepared genomic DNA plate and 13 μL of a PCR master-mix (10 μL NEB Next Q5 Hot Start HiFi PCR Master Mix [NEB M0543L], 2.96 μL nuclease free water, 0.04 μL SYBR Green I 100× [1:100 dilution in nuclease free water of 10,000× SYBR Green I concentrate, ThermoFisher S7567]) into a new 96-well PCR plate. Alongside each set of samples, a no template control (NTC) was performed as a quality control measure utilizing nuclease-free water rather than gDNA as template. The plate was sealed with optically transparent film (Bio-Rad MSB1001), spun down for 30 seconds on a plate microfuge, placed on a qPCR system (Bio-Rad CFX96), and the following PCR program was performed: 98° C. 30 s, 29 cycles: 98° C. 10 s, 65° C. 75 s, 65° C. 5 min, 4° C. infinite. Amplification was observed and stopped while samples remained in exponential amplification (typically 12-15 cycles).

To perform pooling and quality control of the resulting sample amplicons, representative samples and the NTC were assessed on a 2% E-Gel (ThermoFisher G402002 and G6465) for presence of the expected product (164 bp unexpanded CRISPR array product, and expanded products; each new spacer expansion results in addition of ˜61 bp) and no observable product in the NTC. Next, a SYBR Green I plate assay was performed to quantify the relative concentration of amplicon present in each PCR product. Concentrated 10,000× SYBR Green I stock was diluted to a final concentration of 1× in TE, and 198 μL was pipetted with a multichannel pipette into wells of a black optically transparent 96 well plate (ThermoFisher 165305). 2 μL of PCR product was added to each well, and the plate was allowed to incubate in a dark location for 10 minutes. Fluorescence of each well (excitation: 485 nm, emission: 535 nm) was measured on a microplate reader (Tecan Infinite F200), and fluorescence values for individual samples were background subtracted with the fluorescence value of the NTC to control for presence of primers in each PCR. Using this background subtracted fluorescence value, samples were pooled using a Biomek 4000 robot such that equal arbitrary fluorescence units of each sample were present in the final pool.

To remove primers from the pooled product in a manner that did not affect abundance of different amplicon products, the pool was then subjected to gel electrophoresis (2% agarose gel, 100 V) and gel extracted (Promega A9282) from size ranges ˜150 bp to ˜1 kb, and eluted in 30 μL TW in an LoBind tube (Eppendorf 022431021). The amount of DNA present in purified pool was quantified (Qubit dsDNA HS Assay Kit, ThermoFisher Q32854 with Qubit 3.0 Fluorometer, ThermoFisher Q33216) with at least two replicates performed with different pipettes and the average fragment size was quantified on an Agilent Bioanalyzer 2100 with Bioanalyzer High Sensitivity DNA kit (Agilent 5067-4626). The molar concentration of the pool was determined with use of Qubit fluorometric quantification and Bioanalyzer size determination.

### Size-Enrichment of CRISPR Array Libraries

For selected libraries, a size-enrichment protocol was performed to enrich for expanded arrays and deplete unexpanded arrays. SPRI bead-based size selection with AMPureXP beads (Beckman Coulter A63881) was utilized; altering the ratio of AMPureXP added to a particular sample can allow for size selection of a particular library. Rather than performing gel extraction as in the normal library preparation protocol, pooled PCR products were subject to two AmpureXP cleanups with 0.75× ratio of AmpureXP beads to volume of PCR product. These cleanups were performed as per the manufacturer's recommendations with minor modifications: 80% ethanol rather than 70% ethanol, elution into 33 μL TW and removal of 30 μL (to reduce carryover of beads).

The resulting libraries displayed enrichment of larger DNA products which did not appear to be CRISPR arrays and were presumably plasmid or degraded genomic DNA carrying through from the template. This did not alter quality of the resulting library, but to better assess concentration of the library, a qPCR quantification (NEB E7630L) was utilized in addition to fluorometric quantification.

### Sequencing CRISPR Array Libraries

Sequencing was performed on the Illumina MiSeq platform (reagent kits: V3 150 cycle, V2 300 cycle, Micro V2 300 cycle depending on the experiment). All runs included at least a 20% PhiX spike-in (PhiX Sequencing Control V3) which was needed for run completion given relatively low sequence diversity and variable amplicon size. For V3 kits, samples were loaded at 15 μM final concentration, while for V2 kits samples were loaded at 10-12 μM final concentration following the manufacturer's instructions with the following modifications. First, to spike in custom sequencing primers, 6 μL of a 100 μM stock of the CBR1 primer (Table 4) was spiked into well 12 of the reagent cartridge utilizing an extended length tip (Rainin RT-L200XF). Similarly, 6 μL of a 100 μM stock of the CBI1 primer (Table 4) was spiked into well 13 of the reagent cartridge. This spike-in procedure (rather than utilizing custom primer wells) allowed for the PhiX control to be sequenced with primers already present in the standard primer wells. Second, significant amounts of sample may be retained in the sample loading line from run to run, which may result in contamination of samples indexed with similar barcodes. Therefore, after every run an optional template line wash was performed, and where possible unique barcodes were utilized for adjacent runs.

TABLE 4
CRISPR array sequencing primers.

| primer | sequence (5′-3′) |
|---|---|
| CB501 | AATGATACGGCGACCACCGAGATCTACAC TAGATCGC |
| | ctggcttaaaaaatcattaattaataataggttatgtttaga (SEQ ID NO: 9) |
| CB502 | AATGATACGGCGACCACCGAGATCTACAC CTCTCTAT |
| | ctggcttaaaaaatcattaattaataataggttatgtttaga (SEQ ID NO: 10) |
| CB503 | AATGATACGGCGACCACCGAGATCTACAC TATCCTCT |
| | ctggcttaaaaaatcattaattaataataggttatgtttaga (SEQ ID NO: 11) |
| CB504 | AATGATACGGCGACCACCGAGATCTACAC AGAGTAGA |
| | ctggcttaaaaaatcattaattaataataggttatgtttaga (SEQ ID NO: 12) |
| CB505 | AATGATACGGCGACCACCGAGATCTACAC GTAAGGAG |
| | ctggcttaaaaaatcattaattaataataggttatgtttaga (SEQ ID NO: 13) |
| CB506 | AATGATACGGCGACCACCGAGATCTACAC ACTGCATA |
| | ctggcttaaaaaatcattaattaataataggttatgtttaga (SEQ ID NO: 14) |
| CB507 | AATGATACGGCGACCACCGAGATCTACAC AAGGAGTA |
| | ctggcttaaaaaatcattaattaataataggttatgtttaga (SEQ ID NO: 15) |
| CB508 | AATGATACGGCGACCACCGAGATCTACAC CTAAGCCT |
| | ctggcttaaaaaatcattaattaataataggttatgtttaga (SEQ ID NO: 16) |
| CB701 | CAAGCAGAAGACGGCATACGAGAT TCGCCTTA |
| | ggtttgagcgatgatatttgtgct(SEQ ID NO: 17) |
| CB702 | CAAGCAGAAGACGGCATACGAGAT CTAGTACG ggtttgagcgatgatatttgtgct |
| | (SEQ ID NO: 18) |
| CB703 | CAAGCAGAAGACGGCATACGAGAT TTCTGCCT ggtttgagcgatgatatttgtgct |
| | (SEQ ID NO: 19) |
| CB704 | CAAGCAGAAGACGGCATACGAGAT GCTCAGGA ggtttgagcgatgatatttgtgct |
| | (SEQ ID NO: 20) |
| CB705 | CAAGCAGAAGACGGCATACGAGAT AGGAGTCC ggtttgagcgatgatatttgtgct |
| | (SEQ ID NO: 21) |
| CB706 | CAAGCAGAAGACGGCATACGAGAT CATGCCTA ggtttgagcgatgatatttgtgct |
| | (SEQ ID NO: 22) |
| CB707 | CAAGCAGAAGACGGCATACGAGAT GTAGAGAG ggtttgagcgatgatatttgtgct |
| | (SEQ ID NO: 23) |
| CB708 | CAAGCAGAAGACGGCATACGAGAT CCTCTCTG ggtttgagcgatgatatttgtgct |
| | (SEQ ID NO: 24) |
| CB709 | CAAGCAGAAGACGGCATACGAGAT AGCGTAGC ggtttgagcgatgatatttgtgct |
| | (SEQ ID NO: 25) |
| CB710 | CAAGCAGAAGACGGCATACGAGAT CAGCCTCG ggtttgagcgatgatatttgtgct |
| | (SEQ ID NO: 26) |
| CB711 | CAAGCAGAAGACGGCATACGAGAT TGCCTCTT ggtttgagcgatgatatttgtgct |
| | (SEQ ID NO: 27) |
| CB712 | CAAGCAGAAGACGGCATACGAGAT TCCTCTAC ggtttgagcgatgatatttgtgct |
| | (SEQ ID NO: 28) |
| CBR1 | CTGGCTTAAAAAATCATTAATTAATAATAGGTTATGTTTAGAGTGTTCC |
| | CCGCGCCAG (SEQ ID NO: 29) |
| CBI1 | CGGGGATAAACCGAGCACAAATATCATCGCTCAAACC (SEQ ID NO: 30) |

For all samples, underlined bases indicate barcode sequence (derived from Illumina Nextera barcodes).

CRISPR Spacer Extraction and Mapping from Sequencing Data

Raw sequencing reads were analyzed with a custom Python analysis pipeline. Code utilized for sequencing analysis can be found at github.com/ravisheth/trace. Briefly, the pipeline comprised the following steps: (1) raw reads were subjected to spacer extraction, (2) extracted spacers were then mapped against genome and plasmid references to determine their origin, (3) uniquely mapping spacers were determined from mapping results.

To extract spacers (spacer_extraction.py), raw reads were used (given the low error rates of the Illumina platform, and highly structured nature of sequences, filtering of raw sequences was unnecessary). For each read, the beginning 12 bp of the read were checked to ensure that this matched the expected DR sequence. If this criterion was passed, the DR sequence was stripped from the 5′ of read and the remaining sequence was passed into a spacer extraction loop. First, the 5′ of the remaining read sequence was compared to the native genomic first spacer sequence (e.g. end of potential newly acquired spacers); if a match was found whether the read terminated and recorded any spacers extracted, or that the array was unexpanded if no spacers were extracted were considered. If the sequence did not match, as attempt was made to find a DR sequence given different possible spacer lengths, in this case 32-34 bp. If a DR sequence was identified, the spacer was extracted, the spacer and DR sequence were stripped from the 5′ of the read, and the extraction loop was repeated for the remaining sequence. For sequencing runs with 150-159 bp read length, the full DR sequence was utilized during matching, which enabled extraction of up to two new spacers. However, for sequencing runs with 309 bp read length (e.g. maximum possible with 300 cycle reagent kit), only 15 bp of the 5′ of the DR sequence was utilized for matching given read length constraints (using full length DR sequences would only allow for extraction of 4 new spacers). For all multiplexed temporal recordings, the full length DR sequence was utilized to enable differentiation of DR sequences. This extraction routine allowed for high efficiency read extraction (for example, on average >97% of all reads could be extracted without error for each sample).

To map spacers against reference (blast_search.sh), the extracted spacers were searched against reference databases of the genome (NCBI GenBank CP001509.3) and plasmids (as appropriate given the sample) using NCBI BLAST 2.6.0. Extracted spacer files generated by the extraction pipeline were passed to the blastn command, using the flag -evalue 0.0001 to threshold spurious mapping results.

Finally, the resulting BLAST output files were analyzed and spacers mapping to only one reference were determined (unique_spacers.py). This was preferred given that the plasmids may share sequence homology with the reference genome. The resulting uniquely mapping spacers were saved to an output file for further analysis. For analysis of array types frequencies, only arrays with all spacers uniquely mapping to one reference were analyzed.

### Model of CRISPR Array Expansion and Reconstruction of Temporal Input Profiles

A simple model of CRISPR expansion was utilized—a population of CRISPR arrays that undergoes an expansion process during each round of induction was considered. The parameters governing the expansion process are dependent on the identity of the round (if pTrig is activated or not). Specifically:
Each array can undergo expansion with probability pₑₓₚ. The acquired spacer can be:
   A trigger spacer with probability p_{T}
   A reference spacer with probability p_{R}=1−p_{T}
The probability of an array not undergoing expansion is 1−pₑₓₚ

Therefore, for each state (0: no pTrig activation; 1: pTrig activation), two parameters govern the expansion process (pₑₓₚ, p_{T}) for a total of four parameters (p_{exp,0}, p_{T,0}, p_{exp,1}, p_{T,1}) governing the entire model. To determine these parameters, control experiments were utilized as well as the “1111” and “0000” samples; all model parameters can be found in Table 5. To calculate p_{exp,0} and p_{exp,1}, the average proportion of singly expanded arrays after a single round of induction (with and without pTrig activation) was determined from control experiments. To calculate p_{T,0}, the average pTrig incorporation rate across all array lengths and positions (L1 to L5, p1 to p5) from the “0000” sample was used. To calculate p_{T,1}, pTrig incorporation frequencies from the “1111” sample were similarly utilized. However, the pTrig incorporation rate appeared to decrease with array length; likely due to the fact that CRISPR expansion precedes full pTrig activation in the experimental scheme, resulting in highly expanded arrays containing a lower proportion of pTrig spacers (FIG. 13). To account for these differences, an “apparent pTrig incorporation rate” for different array lengths was parameterized based on the “1111” sample by calculating the average pTrig incorporation at each array length. When simulating expected array-type frequencies for different array lengths, the corresponding p^{T,1} for that array length was utilized (e.g. p_{T,1}^{L1} to p_{T,1}^{L5}).

Predicted array-type frequencies were then calculated given a particular temporal input profile and parameterized model. Specifically, all possible array-types were enumerated for a given array-length. The probability of generating each array-type was calculated by enumerating all possible incorporation patterns leading to the array-type (e.g. an array of length 2 during a 3 day temporal input pattern could result from expansion on days {1,2}, {2,3}, or {1,3}) and then analytically calculated the sum of the probabilities of each incorporation pattern. This value was treated as the “global” array-type probability. After all array-type probabilities were calculated, the “global” probabilities for all array-types of a particular length were normalized to unity, resulting in the final predicted array-type frequency vector.

As an example of the model, for a single day of induction (state=1), the probability of an array containing an expanded spacer derived from pTrig (e.g. L1 array, T) is simply p_{exp,1}*p_{T,1}^{L1}. For one day of induction followed by one day of no induction (state=10) the probability of an array containing two expanded spacers derived from pTrig (e.g. L2 array, TT) is simply (p_{exp,1}*p_{T,1}^{L2})*(p_{exp,0}*p_{T,0}). For three days of induction (state=111) the probability of an array containing two expanded spacers, one derived from the genome and the next derived from pTrig (e.g. L2 array, RT) is the sum of all incorporation patterns leading to RT arrays (incorporation on days {1,2}, {2,3}, {1,3}) or:

[p_{exp,1}*(1−p_{T,1}^{L2})]*(p_{exp,1}*p_{T,1}^{L2})*(1−p_{exp,1})+(1−p_{exp,1})*[p_{exp,1}*(1−p_{T,1}^{L2})]*(p_{exp,1}*p_{T,1}^{L2})+[p_{exp,1}*(1−p_{T,1}^{L2})]*(1−p_{exp,1})*(p_{exp,1}*p_{T,1}^{L2})

Array type frequencies can be calculated for any input profile and array-type in a similar manner.

The array-type frequencies calculated from the model were then used to classify the observed data. The Euclidean distance between observed array-type frequencies and predicted array-type frequencies was calculated, and the model with minimum distance to the observed data was selected as the predicted temporal input. This procedure can be repeated for different array lengths. To consider multiple array lengths simultaneously, aggregate array-type vectors were constructed by concatenating array-type vectors of different array lengths of interest (both observed and model) and the same procedure was used to calculate distance and predict temporal inputs.

### Population Lineage Reconstruction Using CRISPR Array Information

To perform lineage reconstruction, genomic spacers within L1 arrays for the 16 4-day temporal recording samples were identified (pooled from enriched and unenriched samples). Genomic spacers were utilized as they contain the highest sequence diversity, and L1 arrays were utilized given that they were observed with the highest frequencies in populations. These spacers were randomly subsampled for each sample to the minimum number of spacers detected (14,715). The location that each spacer mapped to on the reference genome was utilized as the identity of the spacer; the Jaccard distance between two samples (e.g. 1−proportion of unique spacers in a sample shared with another sample) was calculated for all samples in a pairwise fashion. This 16×16 distance matrix was then utilized for lineage reconstruction using the Fitch-Margoliash method (W. M. Fitch, E. Margoliash, Science. 155, 279-284 (1967), incorporated herein by reference in its entirety). Specifically, a tool implementing the PHYLIP program was utilized with default settings (trex.uqam.ca/index.php?action=phylip&app=fitch).

### Multiplexed Recording Analysis and Reconstruction

For all multiplexed temporal recordings, the full length DR sequence was utilized to enable differentiation of DR sequences. Given the strict criteria for DR matching utilized (no more than Hamming distance 2), this allowed for extraction of individual sensors from the CRISPR array populations.

Models were parameterized for each of the three sensors independently. Expansion rates in the absence and presence of signal (p_{exp,0} and p_{exp,1}) were calculated as the average proportion of singly expanded arrays after 1 day for no input and input of all three chemicals (C,T,F) and the same value was utilized for all three sensors. pTrig incorporation rates in the absence of input (p_{T,0}) were calculated for each sensor from profile #1 (e.g. no input throughout the recording) as the average of pTrig spacers at all positions within L1 to L3 arrays. pTrig incorporation rates in the presence of input (p_{T,1}^{L2}, p_{T,1}^{L3}) were calculated for each sensor in a similar manner from profile #2 for L2 and L3 arrays separately. For the CopA sensor, pTrig spacer incorporation was higher when other inducers (T, F) were both present compared to other conditions. Therefore, the pTrig incorporation rate in the presence of input was calculate from profile #6, where the copper was present for three days but other inducers varied. All parameters utilized can be found in Table 5.

TABLE 5
Parameters utilized in CRISPR expansion models

| sensor | state | parameter | value | calculated from |
|---|---|---|---|---|
| LacI | 1 | p_T, L1 | 0.27490 | “1111” sample, pTrig proportion in L1 arrays |
| LacI | 1 | p_T, L2 | 0.24570 | “1111” sample, average of pTrig proportion |
| | | | | in L2 arrays |
| LacI | 1 | p_T, L3 | 0.22020 | “1111” sample, average of pTrig proportion |
| | | | | in L3 arrays |
| LacI | 1 | p_T, L4 | 0.18650 | “1111” sample, average of pTrig proportion |
| | | | | in L4 arrays |
| LacI | 1 | p_T, L5 | 0.18090 | “1111” sample, average of pTrig proportion |
| | | | | in L5 arrays |
| LacI | 0 | p_T | 0.00070 | “0000” sample, average of pTrig proportion |
| | | | | at all positions (L1-L5) |
| LacI | 1 | p_exp | 0.09880 | average proportion singly expanded after |
| | | | | single round (control experiment) |
| LacI | 0 | p_exp | 0.03560 | average proportion singly expanded after |
| | | | | single round (control experiment) |
| CopA | 1 | p_T, L2 | 0.03542 | profile #6, average of pTrig proportion in |
| | | | | CopA sensor in L2 arrays |
| CopA | 1 | p_T, L3 | 0.03092 | profile #6, average of pTrig proportion in |
| | | | | CopA sensor in L3 arrays |
| CopA | 0 | P_T | 0.00107 | profile #1, average of pTrig proportion in |
| | | | | CopA sensor at all array positions L1-L3 |
| TreR | 1 | p_T, L2 | 0.16064 | profile #2, average of pTrig proportion in |
| | | | | TreR sensor in L2 arrays |
| TreR | 1 | p_T, L3 | 0.14790 | profile #2, average of pTrig proportion in |
| | | | | TreR sensor in L3 arrays |
| TreR | 0 | p_T | 0.00065 | profile #1, average of pTrig proportion in |
| | | | | TreR sensor at all array positions L1-L3 |
| GalS | 1 | p_T, L2 | 0.17542 | profile #2, average of pTrig proportion in |
| | | | | GalS sensor in L2 arrays |
| GalS | 1 | p_T, L3 | 0.13966 | profile #2, average of pTrig proportion in |
| | | | | GalS sensor in L3 arrays |
| GalS | 0 | p_T | 0.00306 | profile #1, average of pTrig proportion in |
| | | | | GalS sensor at all array positions L1-L3 |
| CopA/TreR/GalS | 1 | p_exp | 0.10155 | average proportion singly expanded after |
| | | | | one day (across all sensors) with no inducer |
| CopA/TreR/GalS | 0 | p_exp | 0.09829 | average proportion singly expanded after |
| | | | | one day (across all sensors) with all three |
| | | | | inducers |

### Example 2

### CRISPR-Cas9 Genome Editing.

CRISPR systems are found in about 40% of bacteria and 90% of archaea and come in diverse forms. One of the simplest CRISPR systems, Type II spCas9, is found in Streptococcus pyogenes. Targeted DNA cleavage by the Cas9 endonuclease in this system requires a CRISPR RNA (crRNA), the tracrRNA (trans-activating RNA, a small RNA antisense to the CRISPR repeat sequence), and RNase III (which cleaves the tracrRNA:repeat dsRNA to liberate small crRNAs bound to tracrRNA). Cas9 cleaves dsDNA at sites specified by the tracrRNA-crRNA complex and requires an NGG protospacer adjacent motif (PAM) sequence. To further simplify the Type H1 CRISPR system down to two components, it is possible to bypass the need for RNaseIII by designing a guide RNA (gRNA) that mimics the tracrRNA-crRNA complex and targets Cas9 to a specific DNA sequence by complementary base pairing. This unique property of Cas9, which allows the cleavage site to be re-programmed with a small gRNA, has been exploited for genome editing purposes in a wide range of organisms. This technology is highly amenable to high-throughput assays and multiplexing (e.g. several gRNAs can be used at the same time).

Catalytically Dead Cas9 (dCas9).

Although the CRISPR-Cas system cannot be used to introduce site-specific mutations in bacteria generally, as it only cleaves DNA, this system has been used to regulate gene expression by transcriptional interference (CRISPRi). A catalytically dead Cas9 (dCas9) lacking endonuclease activity, but still retaining DNA binding activity, is targeted to a gene of interest by a gRNA, where it binds the DNA to inhibit transcription initiation. Because dCas9 functions as a programmable DNA binding protein, we propose to use dCas9 as a tether for transposase to achieve programmable site-specific transposition. With the recently solved crystal structure of Cas9 bound to a guide RNA and its dsDNA target, Cas9 protein engineering is now more practical. In fact, the Cas9 protein has been successfully split into two pieces that function together as a dimer. Split Cas9 was tagged with eukaryotic nuclear localization signals at the N- and C-termini and with rapamycin inducible FRB and FKBP dimerization domains at an internal disordered linker sequence between the recognition and nuclease lobes. Furthermore, dCas9 has been successfully fused with a zinc finger nuclease Fokd domain, which requires dimerization to cleave DNA, thus producing a dimerization-dependent, programmable nuclease. Fok1-dCas9 was successfully targeted by 2 gRNAs bracketing a target genomic site to cut specifically at that site.

### Bacteroides

Bacteroides species are significant clinical pathogens and are found in most anaerobic infections, with an associated mortality of more than 19%. The bacteria maintain a complex and generally beneficial relationship with the host when retained in the gut, but when they escape this environment they can cause significant pathology, including bacteremia and abscess formation in multiple body sites. Genomic and proteomic analyses have vastly added to our understanding of the manner in which Bacteroides species adapt to, and thrive in, the human gut. A few examples are (i) complex systems to sense and adapt to nutrient availability, (ii) multiple pump systems to expel toxic substances, and (iii) the ability to influence the host immune system so that it controls other (competing) pathogens. B. fragilis, which accounts for only 0.5% of the human colonic flora, is the most commonly isolated anaerobic pathogen due, in part, to its potent virulence factors. Species of the genus Bacteroides have the most antibiotic resistance mechanisms and the highest resistance rates of all anaerobic pathogens. Clinically, Bacteroides species have exhibited increasing resistance to many antibiotics, including cefoxitin, clindamycin, metronidazole, carbapenems, and fluoroquinolones (e.g., gatifloxacin, levofloxacin, and moxifloxacin).

Thus, in certain embodiments, the present methods target Bacteroides species (e.g., B. theta, B. fragilis, B. caccae with a CRISPR-transposon that leads to the directed death of the Bacteroides, as a sort of suicide tool. In additional embodiments, the present methods target Bacteroides with a CRISPR-transposon that leads to the insertion of a desired target gene, such as carbohydrate metabolism genes that allow cells to utilize different energy sources present in the gut and secondarily alter host metabolism.

Clostridium difficile

Pathogenic C. difficile strains produce multiple toxins. The most well-characterized are enterotoxin (Clostridium difficile toxin A) and cytotoxin (Clostridium difficile toxin B), both of which may produce diarrhea and inflammation in infected patients (Clostridium difficile colitis), although their relative contributions have been debated. Toxins A and B are glucosyltransferases that target and inactivate the Rho family of GTPases. Toxin B (cytotoxin) induces actin depolymerization by a mechanism correlated with a decrease in the ADP-ribosylation of the low molecular mass GTP-binding Rho proteins. Another toxin, binary toxin, also has been described, but its role in disease is not fully understood.

Antibiotic treatment of C. diff infections may be difficult, due both to antibiotic resistance and physiological factors of the bacteria (spore formation, protective effects of the pseudomembrane). The emergence of a new, highly toxic strain of C. difficile, resistant to fluoroquinolone antibiotics, such as ciprofloxacin and levofloxacin, said to be causing geographically dispersed outbreaks in North America, was reported in 2005. The U.S. Centers for Disease Control (CDC) in Atlanta warned of the emergence of an epidemic strain with increased virulence, antibiotic resistance, or both.

C. difficile is transmitted from person to person by the fecal-oral route. However, the organism forms heat-resistant spores that are not killed by alcohol-based hand cleansers or routine surface cleaning. Thus, these spores survive in clinical environments for long periods. Because of this, the bacteria may be cultured from almost any surface. Once spores are ingested, their acid-resistance allows them to pass through the stomach unscathed. They germinate and multiply into vegetative cells in the colon upon exposure to bile acids.

A 2015 CDC study estimated that C. diff afflicted almost half a million Americans and caused 29,000 deaths in 2011. The study estimated that 40 percent of cases began in nursing homes or community health care settings, while 24 percent occurred in hospitals.

In certain embodiments, the present methods target Clostridium bacteria such as C. difficile with a CRISPR-transposon that leads to the directed death of the Clostridium, as a sort of suicide tool. In additional embodiments, the present methods target Clostridium with a CRISPR-transposon that leads to the insertion of a desired target gene, such as a gene (e.g. adhesion protein, metabolic pathway, bile resistance) that increases the fitness of gut commensal Clostridia to prevent colonization by pathogens such as C. difficile.

### Enterococcus

Enterococcus is a large genus of lactic acid bacteria of the phylum Firmicutes. Enterococci are Gram-positive cocci that often occur in pairs (diplococci) or short chains, and are difficult to distinguish from streptococci on physical characteristics alone. Two species are common commensal organisms in the intestines of humans: E. faecalis (90-95%) and E. faecium (5-10%). Rare clusters of infections occur with other species, including E. casseliflavus, E. gallinarum, and E. ragffinosus.

Important clinical infections caused by Enterococcus include urinary tract infections, bacteremia, bacterial endocarditis, diverticulitis, and meningitis. Sensitive strains of these bacteria can be treated with ampicillin, penicillin and vancomycin. Urinary tract infections can be treated specifically with nitrofurantoin, even in cases of vancomycin resistance.

From a medical standpoint, an important feature of this genus is the high level of intrinsic antibiotic resistance. Some enterococci are intrinsically resistant to β-lactam-based antibiotics (penicillins, cephalosporins, carbapenems), as well as many aminoglycosides. In the last two decades, particularly virulent strains of Enterococcus that are resistant to vancomycin (vancomycin-resistant Enterococcus, or VRE) have emerged in nosocomial infections of hospitalized patients, especially in the US. VRE may be treated with quinupristin/dalfopristin (Synercid) with response rates around 70%. Tigecycline has also been shown to have antienterococcal activity, as has rifampicin.

Enterococcal meningitis is a rare complication of neurosurgery. It often requires treatment with intravenous or intrathecal vancomycin, yet it is debatable as to whether its use has any impact on outcome: the removal of any neurological devices is a crucial part of the management of these infections.

Thus, in certain embodiments, the present methods target Enterococcal bacteria such as E. faecalis with a CRISPR-transposon that leads to the directed death of the Enterococci, as a sort of suicide tool. In additional embodiments, the present methods target Enterococci with a CRISPR-transposon that leads to the insertion of a desired target gene (e.g., adding genes for adhesion or sugar metabolism to study their roles in determining fitness).

### Example 3: Improved CRISPR-Cas Recording in Clinical Isolates

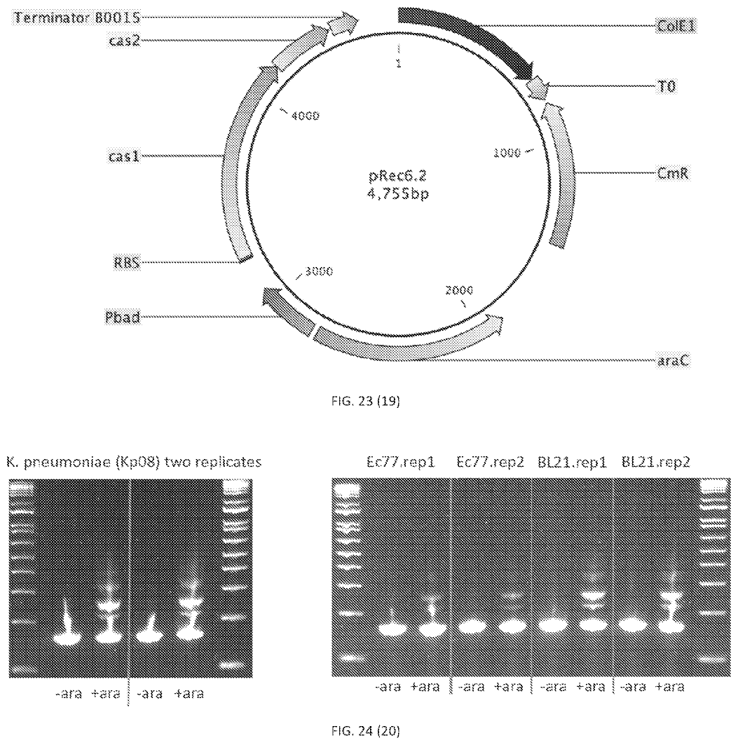

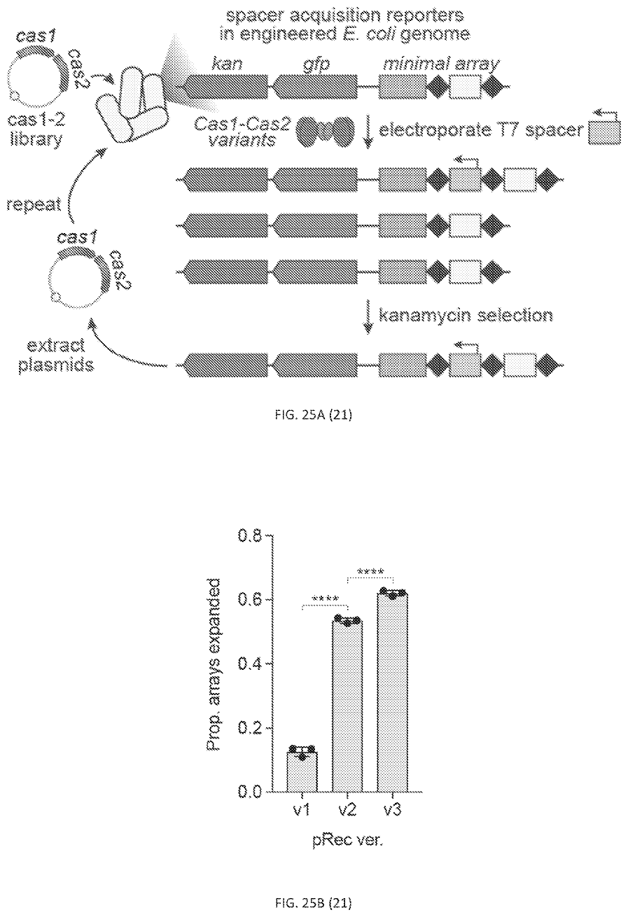

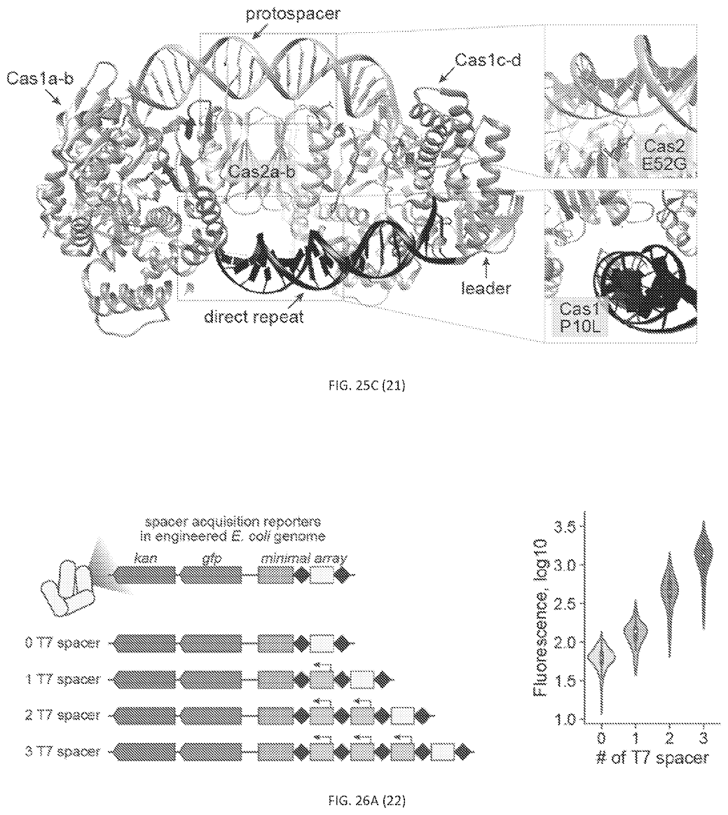

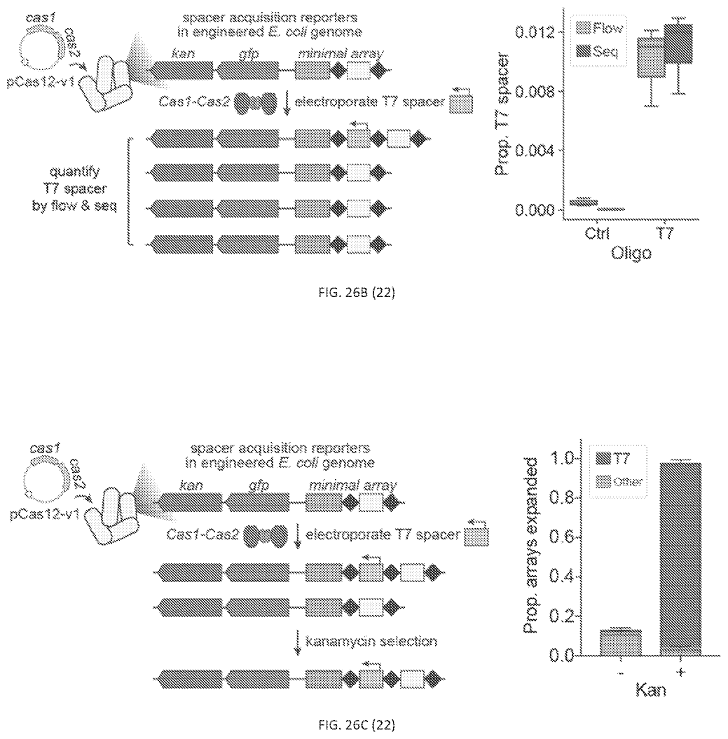

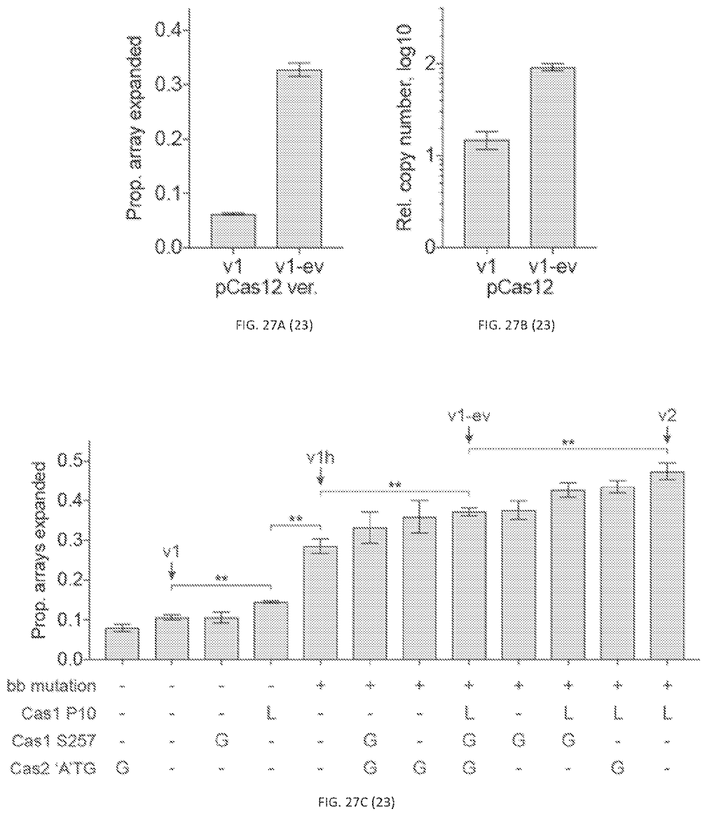

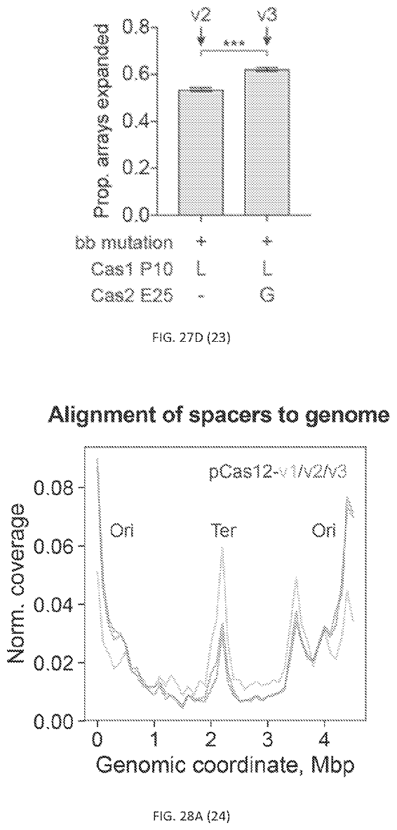

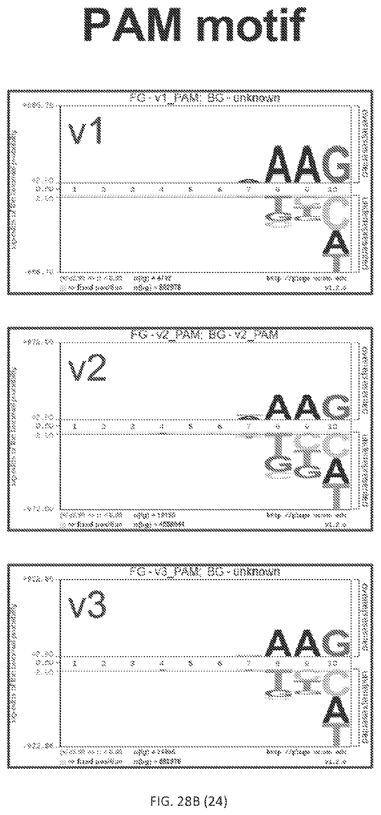

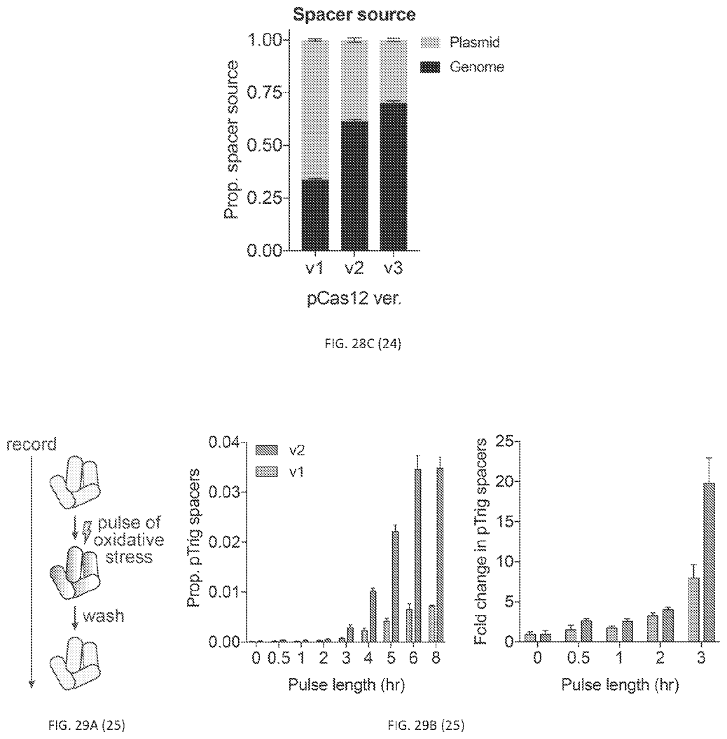

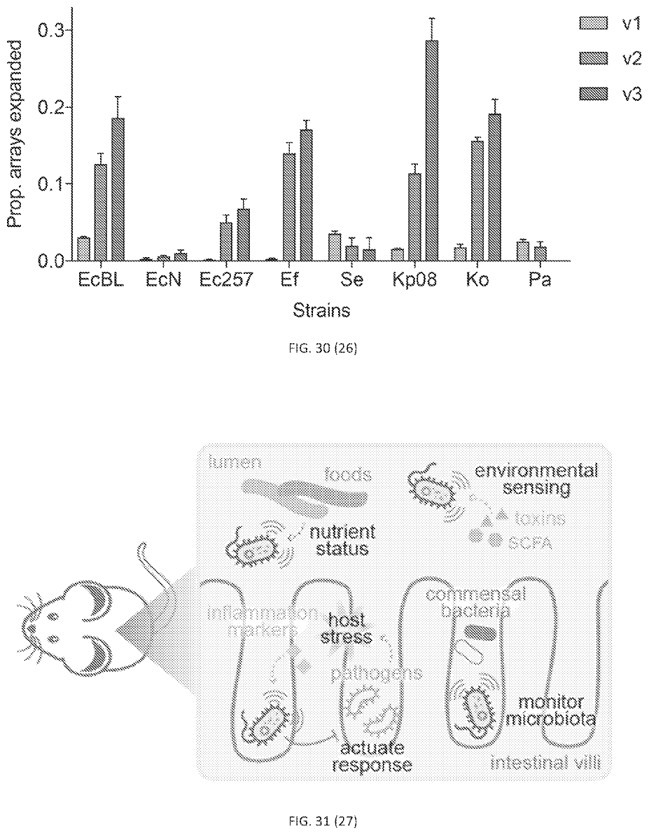

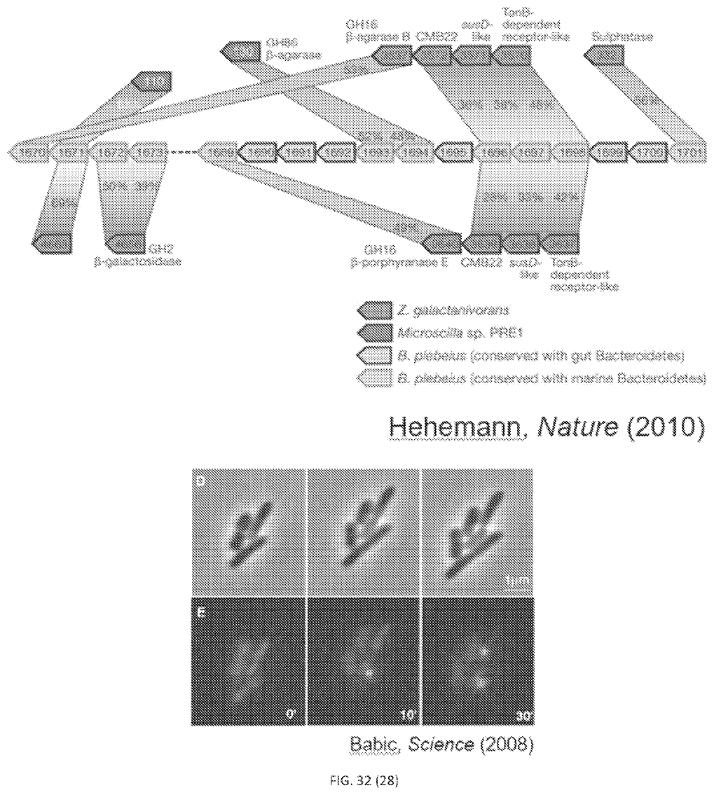

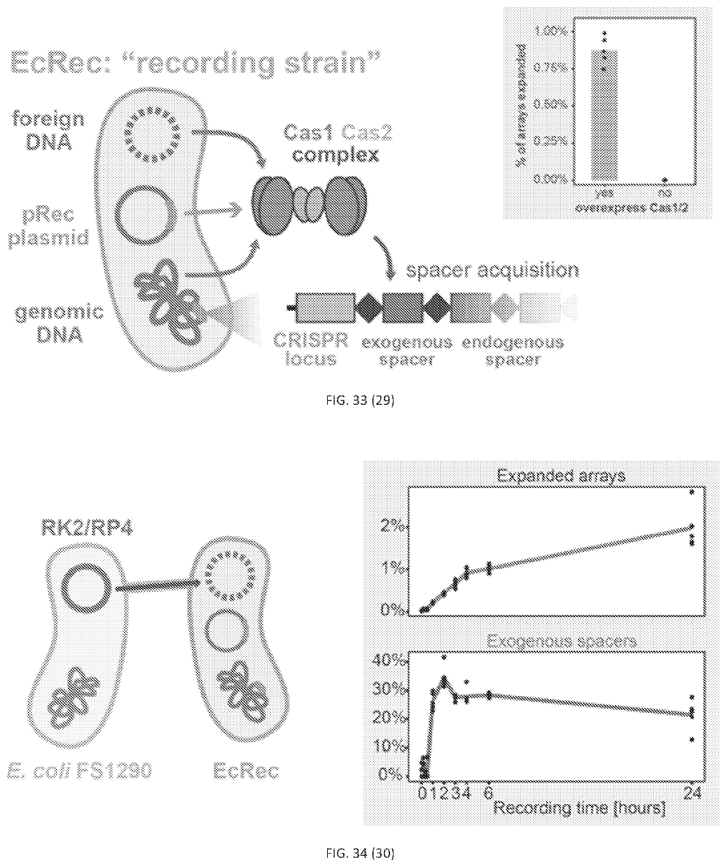

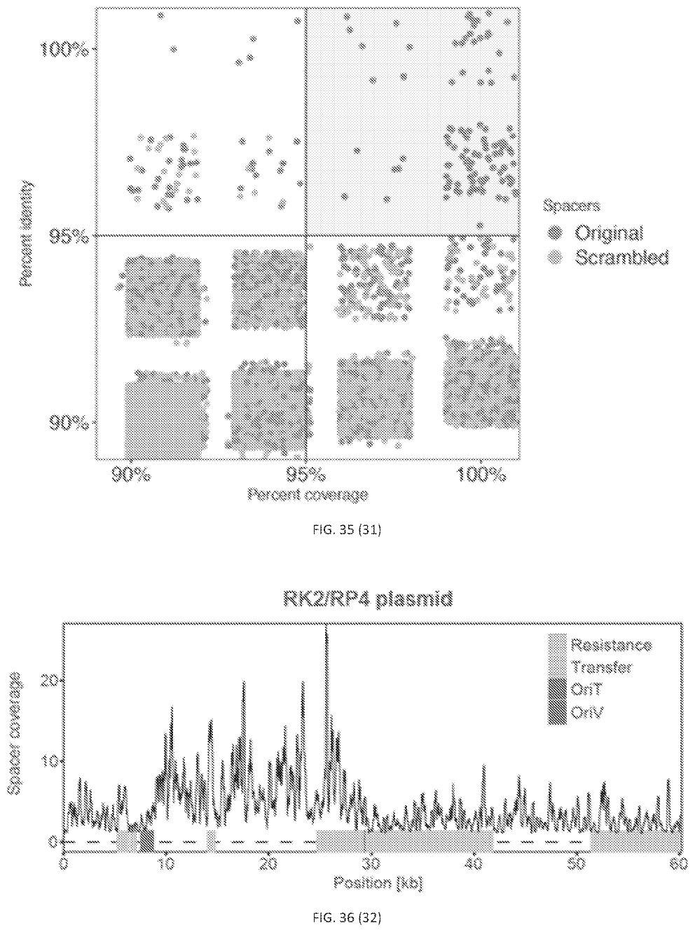

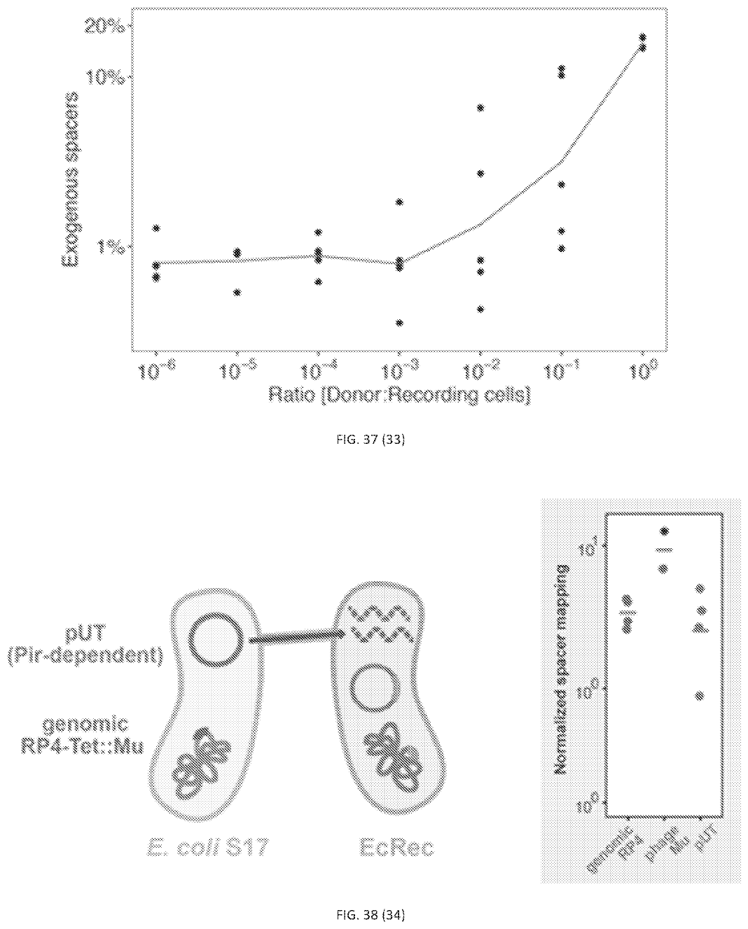

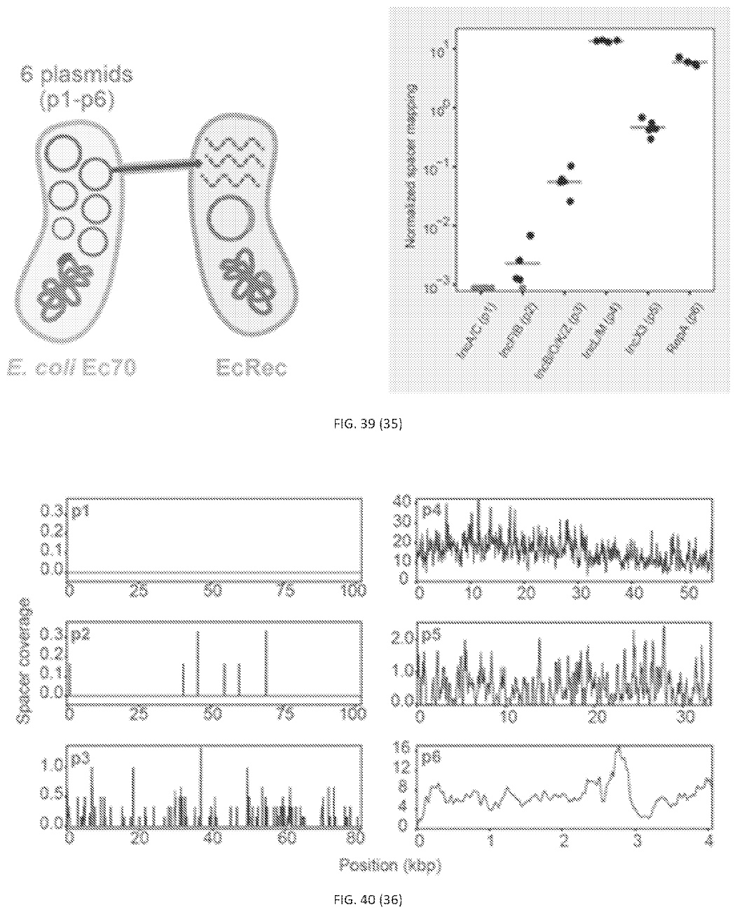

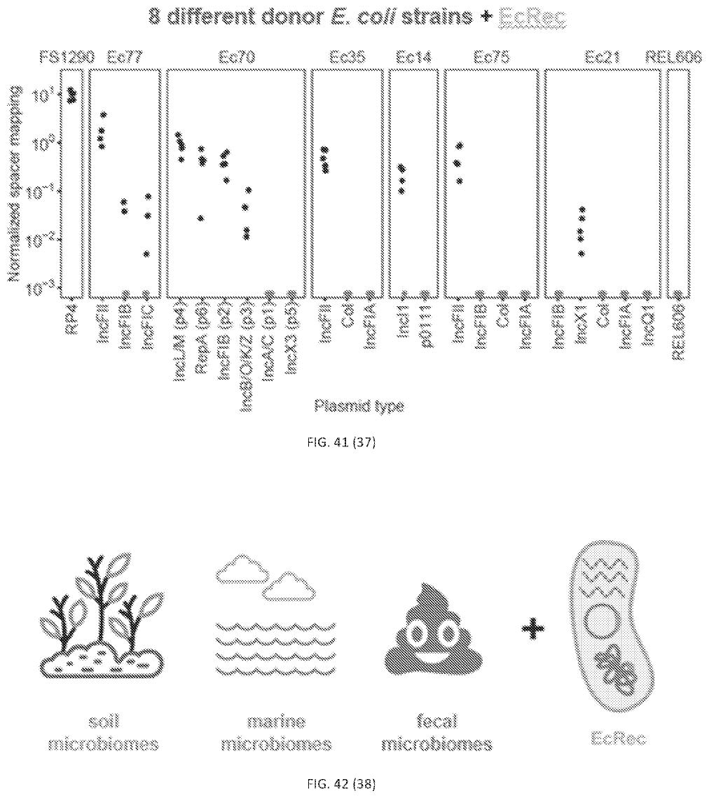

A further plasmid version pRec6 was engineered as shown in FIG. 23, which showed improved recording in an array of different clinical isolates (see FIG. 24). The sequence of the Pbad promoter and the entire plasmid are provided below:

### Sequence of the Pbad Promoter

(SEQ ID NO: 31)
AAGAAACCAATTGTCCATATTGCATCAGACATTGCCGTCACTGCGTCTTT
TACTGGCTCTTCTCGCTAACCAAACCGGTAACCCCGCTTATTAAAAGCAT
TCTGTAACAAAGCGGGACCAAAGCCATGACAAAAACGCGTAACAAAAGTG
TCTATAATCACGGCAGAAAAGTCCACATTGATTATTTGCACGGCGTCACA
CTTTGCTATGCCATAGCATTTTTATCCATAAGATTAGCGGATACTACCTG
ACGCTTTTTATCGCAACTCTCTACTGTTTCTCCAT

Sequence of the pRec plasmid including the Pbad promoter and Cas1 and Cas2 Genes:

LOCUS pRec6.2 4755 bp DNA circular UNA 25-SEP-2017

| DEFINITION | |
|---|---|
| FEATURES | Location/Qualifiers |
| Ori | 1 . . . 653 |
| | /label = “ColE1” |
| | /ApEinfo_revcolor = “#d59687” |
| | /ApEinfo_fwdcolor = “#d59687” |
| primer | 684 . . . 703 |
| | /label = “pRec6.woRep_F″” |
| terminator | 688 . . . 703 |
| | /label = “T0” |
| | /ApEinfo_revcolor = “#ff0517” |
| | /ApEinfo_fwdcolor = “#ff0517” |
| CDS | complement (810 . . . 1469) |
| | /label = “CmR” |
| | /ApEinfo_revcolor = “#ffef86” |
| | /ApEinfo_fwdcolor = “#ffef86” |
| primer | 1464 . . . 1483 |
| | /label = “seq.US.cas12.araC” |
| primer | complement (1662 . . . 1678) |
| | /label = “pRec04: ori: chl_R” |
| CDS | complement (1878 . . . 2756) |
| | /label = “araC” |
| promoter | 2783 . . . 3067 |
| | /label = “Pbad” |
| RBS | 3218 . . . 3223 |
| CDS | 3231 . . . 4148 |
| | /label = “cas1” |
| CDS | 4150 . . . 4434 |
| | /label = “cas2” |
| terminator | 4447 . . . 4575 |
| | /1abel = “Terminator B0015” |
| primer | 4585 . . . 4602 |
| | /label = “CM_pRS600_bb_F” |
| ORIGIN | |
| 1 | GGCCGCGTTG CTGGCGTTTT TCCATAGGCT CCGCCCCCCT GACGAGCATC ACAAAAATCG |
| 61 | ACGCTCAAGT CAGAGGTGGC GAAACCCGAC AGGACTATAA AGATACCAGG CGTTTCCCCC |
| 121 | TGGAAGCTCC CTCGTGCGCT CTCCTGTTCC GACCCTGCCG CTTACCGGAT ACCTGTCCGC |
| 181 | CTTTCTCCCT TCGGGAAGCG TGGCGCTTTC TCAATGCTCA CGCTGTAGGT ATCTCAGTTC |
| 241 | GGTGTAGGTC GTTCGCTCCA AGCTGGGCTG TGTGCACGAA CCCCCCGTTC AGCCCGACCG |
| 301 | CTGCGCCTTA TCCGGTAACT ATCGTCTTGA GTCCAACCCG GTAAGACACG ACTTATCGCC |
| 361 | ACTGGCAGCA GCCACTGGTA ACAGGATTAG CAGAGCGAGG TATGTAGGCG GTGCTACAGA |
| 421 | GTTCTTGAAG TGGTGGCCTA ACTACGGCTA CACTAGAAGG ACAGTATTTG GTATCTGCGC |
| 481 | TCTGCTGAAG CCAGTTACCT TCGGAAAAAG AGTTGGTAGC TCTTGATCCG GCAAACAAAC |
| 541 | CACCGCTGGT AGCGGTGGTT TTTTTGTTTG CAAGCAGCAG ATTACGCGCA GAAAAAAAGG |
| 601 | ATCTCAAGAA GATCCTTTGA TCTTTTCTAC GGGGTCTGAC GCTCAGTGGA ACGAAAACTC |
| 661 | ACGTTAAGGG ATTTTGGTCA TGACTAGTGC TTGGATTCTC ACCAATAAAA AACGCCCGGC |
| 721 | GGCAACCGAG CGTTCTGAAC AAATCCAGAT GGAGTTCTGA GGTCATTACT GGATCTATCA |
| 781 | ACAGGAGTCC AAGCGAGCTC GATATCAAAT TACGCCCCGC CCTGCCACTC ATCGCAGTAC |
| 841 | TGTTGTAATT CATTAAGCAT TCTGCCGACA TGGAAGCCAT CACAGACGGC ATGATGAACC |
| 901 | TGAATCGCCA GCGGCATCAG CACCTTGTCG CCTTGCGTAT AATATTTGCC CATGGTGAAA |
| 961 | ACGGGGGCGA AGAAGTTGTC CATATTGGCC ACGTTTAAAT CAAAACTGGT GAAACTCACC |
| 1021 | CAGGGATTGG CTGAGACGAA AAACATATTC TCAATAAACC CTTTAGGGAA ATAGGCCAGG |
| 1081 | TTTTCACCGT AACACGCCAC ATCTTGCGAA TATATGTGTA GAAACTGCCG GAAATCGTCG |
| 1141 | TGGTATTCAC TCCAGAGCGA TGAAAACGTT TCAGTTTGCT CATGGAAAAC GGTGTAACAA |
| 1201 | GGGTGAACAC TATCCCATAT CACCAGCTCA CCGTCTTTCA TTGCCATACG AAATTCCGGA |
| 1261 | TGAGCATTCA TCAGGCGGGC AAGAATGTGA ATAAAGGCCG GATAAAACTT GTGCTTATTT |
| 1321 | TTCTTTACGG TCTTTAAAAA GGCCGTAATA TCCAGCTGAA CGGTCTGGTT ATAGGTACAT |
| 1381 | TGAGCAACTG ACTGAAATGC CTCAAAATGT TCTTTACGAT GCCATTGGGA TATATCAACG |
| 1441 | GTGGTATATC CAGTGATTTT TTTCTCCATT TTAGCTTCCT TAGCTCCTGA AAATCTCGAT |
| 1501 | AACTCAAAAA ATACGCCCGG TAGTGATCTT ATTTCATTAT GGTGAAAGTT GGAACCTCTT |
| 1561 | ACGTGCCGAT CAACGTCTCA TTTTCGCCAG ATATCGACGT CTAAGAAACC ATTATTATCA |
| 1621 | TGACATTAAC CTATAAAAAT AGGCGTATCA CGAGGCCCTT TCGTCTTCAC CTCGAGTCGG |
| 1681 | TGATGTCGGC GATATAGGCG CCAGCAACCG CACCTGTGGC GCCGGTGATG CCGGCCACGA |
| 1741 | TGCGTCCGGC GTAGAGGATC TGCTCATGTT TGACAGCTTA TCATCGATGC ATAATGTGCC |
| 1801 | TGTCAAATGG ACGAAGCAGG GATTCTGCAA ACCCTATGCT ACTCCGTCAA GCCGTCAATT |
| 1861 | GTCTGATTCG TTACCAATTA TGACAACTTG ACGGCTACAT CATTCACTTT TTCTTCACAA |
| 1921 | CCGGCACGGA ACTCGCTCGG GCTGGCCCCG GTGCATTTTT TAAATACCCG CGAGAAATAG |
| 1981 | AGTTGATCGT CAAAACCAAC ATTGCGACCG ACGGTGGCGA TAGGCATCCG GGTGGTGCTC |
| 2041 | AAAAGCAGCT TCGCCTGGCT GATACGTTGG TCCTCGCGCC AGCTTAAGAC GCTAATCCCT |
| 2101 | AACTGCTGGC GGAAAAGATG TGACAGACGC GACGGCGACA AGCAAACATG CTGTGCGACG |
| 2161 | CTGGCGATAT CAAAATTGCT GTCTGCCAGG TGATCGCTGA TGTACTGACA AGCCTCGCGT |
| 2221 | ACCCGATTAT CCATCGGTGG ATGGAGCCAC TCGTTAATCG CTTCCATGCG CCGCAGTAAC |
| 2281 | AATTGCTCAA GCAGATTTAT CGCCAGCAGC TCCGAATAGC GCCCTTCCCC TTGCCCGGCG |
| 2341 | TTAATGATTT GCCCAAACAG GTCGCTGAAA TGCGGCTGGT GCGCTTCATC CGGGCGAAAG |
| 2401 | AACCCCGTAT TGGCAAATAT TGACGGCCAG TTAAGCCATT CATGCCAGTA GGCGCGCGGA |
| 2461 | CGAAAGTAAA CCCACTGGTG ATACCATTCG CGAGCCTCCG GATGACGACC GTAGTGATGA |
| 2521 | ATCTCTCCTG GCGGGAACAG CAAAATATCA CCCGGTCGGC AAACAAATTC TCGTCCCTGA |
| 2581 | TTTTTCACCA CCCCCTGACC GCGAATGGTG AGATTGAGAA TATAACCTTT CATTCCCAGC |
| 2641 | GGTCGGTCGA TAAAAAAATC GAGATAACCG TTGGCCTCAA TCGGCGTTAA ACCCGCCACC |
| 2701 | AGATGGGCAT TAAACGAGTA TCCCGGCAGC AGGGGATCAT TTTGCGCTTC AGCCATACTT |
| 2761 | TTCATACTCC CGCCATTCAG AGAAGAAACC AATTGTCCAT ATTGCATCAG ACATTGCCGT |
| 2821 | CACTGCGTCT TTTACTGGCT CTTCTCGCTA ACCAAACCGG TAACCCCGCT TATTAAAAGC |
| 2881 | ATTCTGTAAC AAAGCGGGAC CAAAGCCATG ACAAAAACGC GTAACAAAAG TGTCTATAAT |
| 2941 | CACGGCAGAA AAGTCCACAT TGATTATTTG CACGGCGTCA CACTTTGCTA TGCCATAGCA |
| 3001 | TTTTTATCCA TAAGATTAGC GGATACTACC TGACGCTTTT TATCGCAACT CTCTACTGTT |
| 3061 | TCTCCATACC CGTTTTTTTG GGCTAGAAAT AATTTTGTTT AACTTTAAGA AGGAGATATA |
| 3121 | CATATGCGGG GTTCTCAACA TCATCATCAT CATGGTATGG CTAGCATGAC TGGTGGACAG |
| 3181 | CAAATGGGTC GGGATCTGTA CGAGAACCTG TACTTCCAGG AGGACGCCTT ATGACCTGGC |
| 3241 | TTCCCCTTAA TCCCATTCCA CTCAAAGATC GCGTCTCCAT GATCTTTCTG CAATATGGGC |
| 3301 | AGATCGATGT AATAGATGGC GCGTTTGTAC TTATCGACAA GACAGGGATC CGCACTCATA |
| 3361 | TTCCTGTTGG CTCGGTTGCC TGCATCATGC TGGAACCTGG TACACGGGTT TCGCATGCAG |
| 3421 | CTGTACGCCT GGCTGCGCAA GTTGGAACAT TGTTGGTATG GGTGGGGGAA GCGGGCGTTC |
| 3481 | GTGTTTATGC TTCTGGTCAG CCTGGAGGTG CGCGTTCAGA TAAGCTGCTC TATCAGGCAA |
| 3541 | AACTTGCTCT GGATGAAGAT TTGCGTCTGA AGGTCGTACG TAAAATGTTT GAACTTCGGT |
| 3601 | TTGGAGAACC TGCGCCTGCC CGGCGCTCCG TAGAGCAACT CAGAGGTATA GAAGGCAGTC |
| 3661 | GCGTGCGGGC AACCTACGCA CTTCTGGCGA AGCAATACGG CGTGACATGG AATGGACGTC |
| 3721 | GCTACGATCC GAAAGACTGG GAAAAGGGCG ATACGATCAA CCAATGCATT AGCGCTGCAA |
| 3781 | CTTCCTGTTT ATACGGCGTA ACTGAAGCGG CGATACTTGC AGCTGGTTAT GCACCAGCTA |
| 3841 | TTGGGTTTGT GCATACAGGA AAGCCTCTTT CCTTTGTTTA CGATATTGCA GACATCATTA |
| 3901 | AATTTGACAC TGTTGTACCG AAAGCTTTTG AGATAGCGCG TCGTAACCCT GGTGAGCCGG |
| 3961 | ACCGGGAAGT CCGTTTGGCG TGCAGGGATA TTTTTCGCAG TAGTAAAACA TTAGCCAAAT |
| 4021 | TGATTCCGCT TATAGAGGAC GTGCTTGCCG CTGGAGAAAT ACAACCGCCG GCCCCACCTG |
| 4081 | AAGATGCACA GCCTGTTGCC ATTCCGCTTC CTGTTTCACT GGGAGATGCA GGCCATCGGA |
| 4141 | GTAGCTGAAA TGAGTATGTT GGTCGTGGTC ACTGAAAATG TACCTCCGCG CTTACGAGGC |
| 4201 | AGATTAGCCA TCTGGTTGTT GGAGGTACGT GCAGGGGTAT ATGTAGGTGA TGTATCCGCA |
| 4261 | AAAATTCGTG AAATGATCTG GGAACAAATA GCTGGACTGG CGGAAGAAGG CAATGTAGTG |
| 4321 | ATGGCATGGG CAACGAATAC GGAAACGGGA TTTGAGTTCC AGACATTTGG GTTAAACAGG |
| 4381 | CGTACCCCGG TAGATTTGGA TGGTTTAAGG TTGGTGTCTT TTTTACCTGT TTGATAATAA |
| 4441 | TCTAGACCAG GCATCAAATA AAACGAAAGG CTCAGTCGAA AGACTGGGCC TTTCGTTTTA |
| 4501 | TCTGTTGTTT GTCGGTGAAC GCTCTCTACT AGAGTGACAC TGGCTCACCT TCGGGTGGGC |
| 4561 | CTTTCTGCGT TTATAGGTAC CCGTTGAGAG AAGATTTTCA GCCTGATACA GATTAAATCT |
| 4621 | AGACCTAGGC GTTCGGCTGC GGCGAGCGGT ATCAGCTCAC TCAAAGGCGG TAATACGGTT |
| 4681 | ATCCACAGAA TCAGGGGATA ACGCAGGAAA GAACATGTGA GCAAAAGGCC AGCAAAAGGC |
| 4741 | CAGGAACCGT AAAAA (SEQ ID NO: 32) |

Many modifications and variations of this invention can be made without departing from its spirit and scope, as will be apparent to those skilled in the art. The invention is defined by the terms of the appended claims, along with the full scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. The foregoing written specification is considered to be sufficient to enable one skilled in the art to practice the invention. Various modifications of the invention in addition to those shown and described herein will become apparent to those skilled in the art from the foregoing description and fall within the scope of the appended claims.

1. A method of recording a temporal biological signal in a cell, comprising:
exposing the cell to a temporal biological signal,
wherein the cell comprises a trigger nucleic acid and a CRISPR-Cas system, wherein the CRISPR-Cas system comprises a CRISPR array nucleic acid sequence, wherein the trigger nucleic acid comprises at least one oligonucleotide spacer,
wherein presence and/or strength of the temporal biological signal correlates with an abundance of the oligonucleotide spacer,
wherein the CRISPR-Cas system unidirectionally inserts the oligonucleotide spacer into the CRISPR array nucleic acid sequence, and wherein the abundance of the oligonucleotide spacers correlates with a frequency of the oligonucleotide spacer inserted into the CRISPR array nucleic acid sequence, wherein the CRISPR-Cas system comprises an expression construct comprising a nucleic acid sequence encoding Cas1, a nucleic acid sequence encoding Cas2, and a promoter upstream of the Cas1 nucleic acid sequence and Cas2 nucleic acid for driving expression thereof, wherein the promoter is optionally Pbad.

2. The method of claim 1, wherein Cas1 comprises Cas1 (V2).

3. The method of claims 1 or 2, wherein Cas2 comprises Cas2 (V3).

4. The method of any of claims 1-3, wherein a copy number of the trigger nucleic acid is increased by presence and/or strength of a temporal biological signal.

5. The method of any of claims 1-4, wherein the trigger nucleic acid is a plasmid.

6. The method of any of claims 1-5, wherein the expression construct resides on a plasmid.

7. The method of any of claims 1-5, wherein the CRISPR array nucleic acid sequence is integrated into the genome of the cell.

8. The method of any of claims 1-7, wherein the cell is a prokaryotic cell or a eukaryotic cell.

9. The method of claim 8, wherein the prokaryotic cell is a bacterial cell.

10. The method of claim 9, wherein the bacterial cell is Escherichia coli.

11. The method of claim 8, wherein the eukaryotic cell is a yeast cell, plant cell or a mammalian cell.

12. The method of claim 11, wherein the mammalian cell is a human cell.

13. The method of any of claims 1-6 or 8-12, wherein the CRISPR array nucleic acid sequence resides on a plasmid.

14. The method of any of claims 1-13, wherein the signal is a gene expression signal, a metabolite/substance concentration signal, a photo-activated signal, a light-induced signal, a transcriptional signal, a molecular interaction signal, a receptor modulation signal, an electrical signal, and/or an environment signal.

15. The method of claims 1-14, wherein the recorded temporal biological signal is reconstructed.

16. The method of claim 15, wherein the reconstructing is by sequencing the CRISPR array nucleic acid sequence.

17. The method of claim 16, wherein the sequencing determines sequence and order of inserted oligonucleotide spacers in the CRISPR array nucleic acid sequence.

18. A method of recording a plurality of temporal biological signals in cells, comprising:
a. mixing a plurality of populations of cells to generate mixed cells, each population of cells comprising a trigger nucleic acid and a CRISPR-Cas system, wherein the CRISPR-Cas system comprises an CRISPR array nucleic acid sequence, wherein the trigger nucleic acid comprises one or more oligonucleotide spacers, wherein the oligonucleotide spacers in different populations of cells differ; and
b. exposing the mixed cells to a plurality of temporal biological signals,
wherein presence and/or strength of each temporal biological signal correlates with an abundance of a corresponding oligonucleotide spacer;
wherein the CRISPR-Cas system unidirectionally inserts the oligonucleotide spacer into the CRISPR array nucleic acid sequence, wherein the abundances of the oligonucleotide spacers correlate with frequencies of the oligonucleotide spacers inserted into the CRISPR array nucleic acid sequence; and wherein the CRISPR-Cas system comprises an expression construct comprising a nucleic acid sequence encoding Cas1, a nucleic acid sequence encoding Cas2, and a promoter upstream of the Cas1 nucleic acid sequence and Cas2 nucleic acid for driving expression thereof, wherein the promoter is optionally Pbad.

19. The method of claim 18, wherein the oligonucleotide spacers are barcoded via a nucleic acid sequence of a direct repeat (DR) of the CRISPR array nucleic acid sequence.

20. The method of claims 18 or 19, wherein a copy number of the trigger nucleic acid is increased by presence and/or strength of a temporal biological signal.

21. The method of any of claims 18-20, wherein the trigger nucleic acid is a plasmid.

22. The method of any of claims 18-21, wherein the cell is a prokaryotic cell or a eukaryotic cell.

23. The method of claim 22, wherein the prokaryotic cell is a bacterial cell.

24. The method of claim 23, wherein the bacterial cell is Escherichia coli.

25. The method of claim 22, wherein the eukaryotic cell is a yeast cell, plant cell or a mammalian cell.

26. The method of claim 25, wherein the mammalian cell is a human cell.

27. The method of any of claims 18-26, wherein the CRISPR array nucleic acid sequence resides in a genomic DNA of the cell or on a plasmid.

28. The method of any of claims 18-27, wherein the signal is a gene expression signal, a metabolite/substance concentration signal, a photo-activated signal, a light-induced signal, a transcriptional signal, a molecular interaction signal, a receptor modulation signal, an electrical signal, and/or an environment signal.

29. The method of any of claims 18-28, wherein the recorded temporal biological signal is reconstructed.

30. The method of claim 29, wherein the reconstructing is by sequencing the CRISPR array nucleic acid sequence.

31. The method of claim 30, wherein the sequencing determines sequence and order of inserted oligonucleotide spacers in the CRISPR array nucleic acid sequence.

32. A biological recording system comprising:
a cell comprising a trigger nucleic acid and a CRISPR-Cas system, wherein the CRISPR-Cas system comprises an CRISPR array nucleic acid sequence, wherein the trigger nucleic acid comprises at least one oligonucleotide spacer,
wherein an abundance of the oligonucleotide spacer is increased by presence and/or strength of a temporal biological signal, wherein the CRISPR-Cas system unidirectionally inserts the oligonucleotide spacer into the CRISPR array nucleic acid sequence, wherein the abundance of the oligonucleotide spacer correlates with a frequency of the oligonucleotide spacer inserted into the CRISPR array nucleic acid sequence; and wherein the CRISPR-Cas system comprises an expression construct comprising a nucleic acid sequence encoding Cas1, a nucleic acid sequence encoding Cas2, and a promoter upstream of the Cas1 nucleic acid sequence and Cas2 nucleic acid for driving expression thereof, wherein the promoter is optionally Pbad.

33. A kit comprising the biological recording system of claim 32.

34. A composition comprising the biological recording system of claim 32.

35. The method of any of claims 1-16, wherein the CRISPR-Cas system inserts one or more reference spacers into the CRISPR array nucleic acid sequence.

36. The method of claim 35, wherein the reference spacers are derived from the cell's genome and/or one or more plasmids in the cell.

37. A method of reconstructing lineage of cells, comprising:
analyzing a sequence identity of a plurality of reference spacers inserted into a CRISPR array nucleic acid sequence in the cells, wherein the cells comprise a CRISPR-Cas system comprising the CRISPR array nucleic acid sequence.

38. The method of claim 36, wherein the reference spacers are derived from the cells' genome and/or one or more plasmids in the cells.

39. A biological recording system comprising:
an engineered, non-naturally occurring cell comprising a trigger nucleic acid and a CRISPR-Cas system, wherein the CRISPR-Cas system comprises an CRISPR array nucleic acid sequence, wherein the trigger nucleic acid comprises at least one oligonucleotide spacer,
wherein an abundance of the oligonucleotide spacer is increased by presence and/or strength of a temporal biological signal, wherein the CRISPR-Cas system unidirectionally inserts the oligonucleotide spacer into the CRISPR array nucleic acid sequence, wherein the abundance of the oligonucleotide spacer correlates with a frequency of the oligonucleotide spacer inserted into the CRISPR array nucleic acid sequence; and wherein the CRISPR-Cas system comprises an expression construct comprising a nucleic acid sequence encoding Cas1, a nucleic acid sequence encoding Cas2, and a promoter upstream of the Cas1 nucleic acid sequence and Cas2 nucleic acid for driving expression thereof, wherein the promoter is optionally Pbad.

40. An expression construct comprising a Cas1 encoding nucleic acid sequence, a Cas2 encoding nucleic acid sequence, and an upstream promoter driving expression of the Cas1 and Cas2 encoding nucleic acid sequences, wherein the Cas1 is Cas1 (V2) and/or Cas2 is Cas2 (V3).

41. The expression construct of claim 40, wherein the expression construct resides on a plasmid.

42. An expression construct comprising a Cas1 encoding nucleic acid sequence, a Cas2 encoding nucleic acid sequence, and an upstream Pbad promoter driving expression of the Cas1 and Cas2 encoding nucleic acid sequences.
